SEQUENCE LISTING (65 sequences)

SEQ ID NO: 1 - protein, 812 aa, Murinae sp.
MDHKEVILLFLLLLKPGQGDSLDGYISTQGASLFSLTKKQLAAGGVSDCLAKCEGETDFVCRSFQYHSKEQQCVIMAENSKTSSIIRMRDVILFEKRVYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPWCYTTDPSVRWEYCNLKRCSETGGSVVELPTVSQEPSGPSDSETDCMYGNGKDYRGKTAVTAAGTPCQGWAAQEPHRHSIFTPQTNPRADLEKNYCRNPDGDVNGPWCYTTNPRKLYDYCDIPLCASASSFECGKPQVEPKKCPGRVVGGCVANPHSWPWQISLRTRFTGQHFCGGTLIAPEWVLTAAHCLEKSSRPEFYKVILGAHEEYIRGLDVQEISVAKLILEPNNRDIALLKLSRPATITDKVIPACLPSPNYMVADRTICYITGWGETQGTFGAGRLKEAQLPVIENKVCNRVEYLNNRVKSTELCAGQLAGGVDSCQGDSGGPLVCFEKDKYILQGVTSWGLGCARPNKPGVYVRVSRFVDWIEREMRNN

SEQ ID NO: 2 - protein, 339 aa, Murinae sp.
VYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPW

SEQ ID NO: 3 - protein, 339 aa, Homo sapiens
VYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPW

SEQ ID NO: 4 - protein, 339 aa, Rhesus monkey
VYLSECKTGNGKNYRGTMSKTRTGITCQKWSSTSPHRPTFSPATHPSEGLEENYCRNPDNDGQGPWCYTTDPEERFDYCDIPECEDECMHCSGENYDGKISKTMSGLECQAWDSQSPHAHGYIPSKFPNKNLKKNYCRNPDGEPRPWCFTTDPNKRWELCDIPRCTTPPPSSGPTYQCLKGTGENYRGDVAVTVSGHTCHGWSAQTPHTHNRTPENFPCKNLDENYCRNPDGEKAPWCYTTNSQVRWEYCKIPSCESSPVSTEPLDPTAPPELTPVVQECYHGDGQSYRGTSSTTTTGKKCQSWSSMTPHWHEKTPENFPNAGLTMNYCRNPDADKGPW

SEQ ID NO: 5 - protein, 339 aa, Porcine
IYLSECKTGNGKNYRGTTSKTKSGVICQKWSVSSPHIPKYSPEKFPLAGLEENYCRNPDNDEKGPWCYTTDPETRFDYCDIPECEDECMHCSGEHYEGKISKTMSGIECQSWGSQSPHAHGYLPSKFPNKNLKMNYCRNPDGEPRPWCFTTDPNKRWEFCDIPRCTTPPPTSGPTYQCLKGRGENYRGTVSVTASGHTCQRWSAQSPHKHNRTPENFPCKNLEENYCRNPDGETAPWCYTTDSEVRWDYCKIPSCGSSTTSTEHLDAPVPPEQTPVAQDCYRGNGESYRGTSSTTITGRKCQSWVSMTPHRHEKTPGNFPNAGLTMNYCRNPDADKSPW

SEQ ID NO: 6 - protein, 339 aa, Bovine
IYLLECKTGNGQTYRGTTAETKSGVTCQKWSATSPHVPKFSPEKFPLAGLEENYCRNPDNDENGPWCYTTDPDKRYDYCDIPECEDKCMHCSGENYEGKIAKTMSGRDCQAWDSQSPHAHGYIPSKFPNKNLKMNYCRNPDGEPRPWCFTTDPQKRWEFCDIPRCTTPPPSSGPKYQCLKGTGKNYGGTVAVTESGHTCQRWSEQTPHKHNRTPENFPCKNLEENYCRNPDGEKAPWCYTTNSEVRWEYCTIPSCESSPLSTERMDVPVPPEQTPVPQDCYHGNGQSYRGTSSTTITGRKCQSWSSMTPHRHLKTPENYPNAGLTMNYCRNPDADKSPW

SEQ ID NO: 7 - protein, 79 aa, Murinae sp.
CKTGIGNGYRGTMSRTKSGVACQKWGATFPHVPNYSPSTHPNEGLEENYCRNPDNDEQGPWCYTTDPDKRYDYCNIPEC

SEQ ID NO: 8 - protein, 79 aa, Homo sapiens
CKTGNGKNYRGTMSKTKNGITCQKWSSTSPHRPRFSPATHPSEGLEENYCRNPDNDPQGPWCYTTDPEKRYDYCDILEC

SEQ ID NO: 9 - protein, 79 aa, Rhesus monkey
CKTGNGKNYRGTMSKTRTGITCQKWSSTSPHRPTFSPATHPSEGLEENYCRNPDNDGQGPWCYTTDPEERFDYCDIPEC

SEQ ID NO: 10 - protein, 79 aa, Porcine
CKTGNGKNYRGTTSKTKSGVICQKWSVSSPHIPKYSPEKFPLAGLEENYCRNPDNDEKGPWCYTTDPETRFDYCDIPEC

SEQ ID NO: 11 - protein, 79 aa, Bovine
CKTGNGQTYRGTTAETKSGVTCQKWSATSPHVPKFSPEKFPLAGLEENYCRNPDNDENGPWCYTTDPDKRYDYCDIPEC

SEQ ID NO: 12 - protein, 78 aa, Murinae sp.
CMYCSGEKYEGKISKTMSGLDCQAWDSQSPHAHGYIPAKFPSKNLKMNYCHNPDGEPRPWCFTTDPTKRWEYCDIPRC

SEQ ID NO: 13 - protein, 78 aa, Homo sapiens
CMHSSGENYDGKISKTMSGLECQAWDSQSPHAHGYIPSKFPNKNLKKNYCRNPDRELRPWCFTTDPNKRWELCDIPRC

SEQ ID NO: 14 - protein, 78 aa, Rhesus monkey
CMHCSGENYDGKISKTMSGLECQAWDSQSPHAHGYIPSKFPNKNLKKNYCRNPDGEPRPWCFTTDPNKRWELCDIPRC

SEQ ID NO: 15 - protein, 78 aa, Porcine
CMHCSGEHYEGKISKTMSGIECQSWGSQSPHAHGYLPSKFPNKNLKMNYCRNPDGEPRPWCFTTDPNKRWEFCDIPRC

SEQ ID NO: 16 - protein, 78 aa, Bovine
CMHCSGENYEGKIAKTMSGRDCQAWDSQSPHAHGYIPSKFPNKNLKMNYCRNPDGEPRPWCFTTDPQKRWEFCDIPRC

SEQ ID NO: 17 - protein, 78 aa, Murine
CLKGRGENYRGTVSVTVSGKTCQRWSEQTPHRHNRTPENFPCKNLEENYCRNPDGETAPWCYTTDSQLRWEYCEIPSC

SEQ ID NO: 18 - protein, 78 aa, Homo sapiens
CLKGTGENYRGNVAVTVSGHTCQHWSAQTPHTHNRTPENFPSKNLDENYCRNPDGKRAPWCHTTNSQVRWEYCKIPSC

SEQ ID NO: 19 - protein, 78 aa, Rhesus monkey
CLKGTGENYRGDVAVTVSGHTCHGWSAQTPHTHNRTPENFPCKNLDENYCRNPDGEKAPWCYTTNSQVRWEYCKIPSC

SEQ ID NO: 20 - protein, 78 aa, Porcine
CLKGRGENYRGTVSVTASGHTCQRWSAQSPHKHNRTPENFPCKNLEENYCRNPDGETAPWCYTTDSEVRWDYCKIPSC

SEQ ID NO: 21 - protein, 78 aa, Bovine
CLKGTGKNYGGTVAVTESGHTCQRWSEQTPHKHNRTPENFPCKNLEENYCRNPDGEKAPWCYTTNSEVRWEYCTIPSC

SEQ ID NO: 22 - protein, 78 aa, Murinae sp.
CYQSDGQSYRGTSSTTITGKKCQSWAAMFPHRHSKTPENFPDAGLEMNYCRNPDGDKGPWCYTTDPSVRWEYCNLKRC

SEQ ID NO: 23 - protein, 78 aa, Homo sapiens
CYHGDGQSYRGTSSTTTTGKKCQSWSSMTPHRHQKTPENYPNAGLTMNYCRNPDADKGPWCFTTDPSVRWEYCNLKKC

SEQ ID NO: 24 - protein, 168 aa, Murine
CMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSC

SEQ ID NO: 25 - protein, 168 aa, Homo sapiens
CMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSC

SEQ ID NO: 26 - protein, 168 aa, Rhesus monkey
CMHCSGENYDGKISKTMSGLECQAWDSQSPHAHGYIPSKFPNKNLKKNYCRNPDGEPRPWCFTTDPNKRWELCDIPRCTTPPPSSGPTYQCLKGTGENYRGDVAVTVSGHTCHGWSAQTPHTHNRTPENFPCKNLDENYCRNPDGEKAPWCYTTNSQVRWEYCKIPSC

SEQ ID NO: 27 - protein, 168 aa, Porcine
CMHCSGEHYEGKISKTMSGIECQSWGSQSPHAHGYLPSKFPNKNLKMNYCRNPDGEPRPWCFTTDPNKRWEFCDIPRCTTPPPTSGPTYQCLKGRGENYRGTVSVTASGHTCQRWSAQSPHKHNRTPENFPCKNLEENYCRNPDGETAPWCYTTDSEVRWDYCKIPSC

SEQ ID NO: 28 - protein, 168 aa, Bovine
CMHCSGENYEGKIAKTMSGRDCQAWDSQSPHAHGYIPSKFPNKNLKMNYCRNPDGEPRPWCFTTDPQKRWEFCDIPRCTTPPPSSGPKYQCLKGTGKNYGGTVAVTESGHTCQRWSEQTPHKHNRTPENFPCKNLEENYCRNPDGEKAPWCYTTNSEVRWEYCTIPSC

SEQ ID NO: 29 - protein, 250 aa, Murinae sp.
CKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSC

SEQ ID NO: 30 - protein, 250 aa, Homo sapiens
CKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSC

SEQ ID NO: 31 - protein, 250 aa, Rhesus monkey
CKTGNGKNYRGTMSKTRTGITCQKWSSTSPHRPTFSPATHPSEGLEENYCRNPDNDGQGPWCYTTDPEERFDYCDIPECEDECMHCSGENYDGKISKTMSGLECQAWDSQSPHAHGYIPSKFPNKNLKKNYCRNPDGEPRPWCFTTDPNKRWELCDIPRCTTPPPSSGPTYQCLKGTGENYRGDVAVTVSGHTCHGWSAQTPHTHNRTPENFPCKNLDENYCRNPDGEKAPWCYTTNSQVRWEYCKIPSC

SEQ ID NO: 32 - protein, 250 aa, Porcine
CKTGNGKNYRGTTSKTKSGVICQKWSVSSPHIPKYSPEKFPLAGLEENYCRNPDNDEKGPWCYTTDPETRFDYCDIPECEDECMHCSGEHYEGKISKTMSGIECQSWGSQSPHAHGYLPSKFPNKNLKMNYCRNPDGEPRPWCFTTDPNKRWEFCDIPRCTTPPPTSGPTYQCLKGRGENYRGTVSVTASGHTCQRWSAQSPHKHNRTPENFPCKNLEENYCRNPDGETAPWCYTTDSEVRWDYCKIPSC

SEQ ID NO: 33 - protein, 250 aa, Bovine
CKTGNGQTYRGTTAETKSGVTCQKWSATSPHVPKFSPEKFPLAGLEENYCRNPDNDENGPWCYTTDPDKRYDYCDIPECEDKCMHCSGENYEGKIAKTMSGRDCQAWDSQSPHAHGYIPSKFPNKNLKMNYCRNPDGEPRPWCFTTDPQKRWEFCDIPRCTTPPPSSGPKYQCLKGTGKNYGGTVAVTESGHTCQRWSEQTPHKHNRTPENFPCKNLEENYCRNPDGEKAPWCYTTNSEVRWEYCTIPSC

SEQ ID NO: 34 - protein, 160 aa, Murinae sp.
CKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRC

SEQ ID NO: 35 - protein, 160 aa, Homo sapiens
CKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRC

SEQ ID NO: 36 - protein, 160 aa, Rhesus monkey
CKTGNGKNYRGTMSKTRTGITCQKWSSTSPHRPTFSPATHPSEGLEENYCRNPDNDGQGPWCYTTDPEERFDYCDIPECEDECMHCSGENYDGKISKTMSGLECQAWDSQSPHAHGYIPSKFPNKNLKKNYCRNPDGEPRPWCFTTDPNKRWELCDIPRC

SEQ ID NO: 37 - protein, 160 aa, Porcine
CKTGNGKNYRGTTSKTKSGVICQKWSVSSPHIPKYSPEKFPLAGLEENYCRNPDNDEKGPWCYTTDPETRFDYCDIPECEDECMHCSGEHYEGKISKTMSGIECQSWGSQSPHAHGYLPSKFPNKNLKMNYCRNPDGEPRPWCFTTDPNKRWEFCDIPRC

SEQ ID NO: 38 - protein, 160 aa, Bovine
CKTGNGQTYRGTTAETKSGVTCQKWSATSPHVPKFSPEKFPLAGLEENYCRNPDNDENGPWCYTTDPDKRYDYCDIPECEDKCMHCSGENYEGKIAKTMSGRDCQAWDSQSPHAHGYIPSKFPNKNLKMNYCRNPDGEPRPWCFTTDPQKRWEFCDIPRC

SEQ ID NO: 39 - protein, 352 aa, Murinae sp.
CKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPWCYTTDPSVRWEYCNLKRC

SEQ ID NO: 40 - protein, 352 aa, Homo sapiens
CKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKC

SEQ ID NO: 41 - protein, 378 aa, Murinae sp.
LFEKRVYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPWCYTTDPSVRWEYCNLKRCSETGGSVVELPTVSQE

SEQ ID NO: 42 - protein, 378 aa, Homo sapiens
LFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLL

SEQ ID NO: 43 - protein, 20 aa, Murinae sp.
HTHQDFQPVLHLVALNTPLS

SEQ ID NO: 44 - protein, 10 aa, Murinae sp.
MARRASVGTD

SEQ ID NO: 45 - DNA, 900 bp, Homo sapiens
cacagccaccgcgacttccagccggtgctccacctggttgcgctcaacagccccctgtcagtgtcggtggcgctgaaggtcggccacgaggtggaccaacgcgagttgtcgggggacagtggcggcatgcggggcatccgcggggccgacttccagtgcttccagcaggcgcgggccgtgccgccgtacgccccgtaggcgccccggctgaaggtcacgaaggtcgtccgcgcccggcacgggctggcgggcaccttccgcgccttcctgtcctcgcgcctgcaggacctgtacagcatccccgaccgcccgtggaaggcgcggaaggacaggagcgcggacgtcctggacatgtcgtaggtgcgccgtgccgaccgcgcagccgtgcccatcgtcaacctcaaggacgagctgctgtttcacgcggcacggctggcgcgtcggcacgggtagcagttggagttcctgctcgacgacaaacccagctgggaggctctgttctcaggctctgagggtccgctgaagcccggggcacgcatcgggtcgaccctccgagacaagagtccgagactcccaggcgacttcgggccccgtgcgtagttctcctttgacggcaaggacgtcctgaggcaccccacctggccccagaagagcgtgtggcatggctcggaccccaacgggcgcaggctgaccgagagctactgtgagacgtggcggacggtaccgagcctggggttgcccgcgtccgactggctctcgatgacactctgcaccgcctgcgaggctccctcggccacgggccaggcctcctcgctgctggggggcaggctcctggggcagtcacggcgctcgacggtagtgcggatgtagcacgagacgtaactcttgtcgaagtactga

SEQ ID NO: 46 - protein, 184 aa, Murinae sp.
HTHQDFQPVLHLVALNTPLSGGMRGIRGADFQCFQQARAVGLSGTFRAFLSSRLQDLYSIVRRADRGSVPIVNLKDEVLSPSWDSLFSGSQGQVQPGARIFSFDGRDVLRHPAWPQKSVWHGSDPSGRRLMESYCETWRTETTGATGQASSLLSGRLLEQKAASCHNSYIVLCIENSFMTSFSK

SEQ ID NO: 47 - protein, 180 aa, Rhesus monkey
HSHRDFQPVLHLVALNSPLPGGMRGIRGADFQCFQQARAVGLVGTFRAFLSSRLQDLYSIVRRADRAAVPIVNLKDELLFPSWEALFAGSEGPLKPGARIFSFDGKDVLRHPTWPQKSVWHGSDPSGRRLTESYCETWRTESPSVTGQASSLLGGRLLGQNAASCHHAYIVLCIENSFMT

SEQ ID NO: 48 - DNA, 540 bp, Rhesus monkey
cacagccaccgcgacttccagcccgtgctccacctggttgcgctcaatagcccgctgccaggcggcatgcggggcatccgcggggccgacttccagtgcttccagcaggcacgggccgtggggctggtgggcaccttccgtgccttcctgtcctcacggctgcaggacctgtacagcatcgtgcgccgtgccgaccgcgcagccgtgcccatcgtcaacctcaaggatgagctgctgtttcccagctgggaggctttgttcgcaggctctgagggtccgctgaagcccggggcacgcatcttctcctttgacggcaaggacgtcctgaggcaccccacctggccccagaagagcgtgtggcatggctcggaccccagcgggcgcaggctgactgagagctactgcgagacgtggcggacagagtctccctcggtcacaggtcaggcctcctccctgctggggggcaggctcctagggcagaatgccgcaagctgtcaccacgcctatatcgtcctctgcatcgagaacagcttcatgact

SEQ ID NO: 49 - protein, 184 aa, Canine sp.
HTHQDFQPVLHLVALNSPQPGGMRGIRGADFQCFQQARAAGLAGTFRAFLSSRLQDLYSIVRRADRTGVPVVNLRDEVLFPSWEALFSGSEGQLKPGARIFSFDGRDVLQHPAWPRKSVWHGSDPSGRRLTDSYCETWRTEAPAATGQASSLLAGRLLEQEAASCRHAFVVLCIENSVMTSFSK

SEQ ID NO: 50 - DNA, 552 bp, Canine sp.
cacacccaccaggacttccagccggtgctgcacctggtggccctgaacagcccgcagccgggcggcatgcgaggcatccggggagcggacttccagtgcttccagcaggcgcgcgccgcggggctggccggcaccttccgggccttcctgtcgtcgcggctgcaggacctctacagcatcgtgcgccgcgccgaccgcaccggggtgcccgtcgtcaacctcagggacgaggtgctcttccccagctgggaggccttattctcgggctccgagggccagctgaagcccggggcccgcatcttctctttcgacggcagagatgtcctgcagcaccccgcctggccccggaagagcgtgtggcacggctccgaccccagcgggcgccgcctgaccgacagctactgcgagacgtggcggacggaggccccggcggccaccgggcaggcgtcgtcgctgctggcgggcaggctgctggagcaggaggccgcgagctgccgccacgccttcgtggtgctctgcatcgagaacagcgtcatgacctccttctccaag

SEQ ID NO: 51 - DNA, 632 bp, Murinae sp.
catactcatcaggactttcagccagtgctccacctggtggcactgaacacccccctgtctggaggcatgcgtggtatccgtggagcagatttccagtgcttccagcaagcccgagccgtggggctgtcgggcaccttccgggctttcctgtcctctaggctgcaggatctctatagcatcgtgcgccgtgctgaccgggggtctgtgcccatcgtcaacctgaaggacgaggtgctatcttgcaggacctctacagcatcgtgcgccgcgccgaccgcaccggggtgcccgtcgtcaacctcagggacgaggtgctcttccccagctgggaggccttattctcgggctccgagggccagctgaagcccggggcccgcatcttctctttcgacggcagagatgtcctgcagcaccccgcctggccccggaagagcgtgtggcacggctccgaccccagcgggcgccgcctgaccgacagctactgcgagacgtggcggacggaggccccggcggccaccgggcaggcgtcgtcgctgctggcgggcaggctgctggagcaggaggccgcgagctgccgccacgccttcgtggtgctctgcatcgagaacagcgtcatgacctccttctccaag

SEQ ID NO: 52 - protein, 183 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 53 - DNA, 549 bp, Homo sapiens
cacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaag

SEQ ID NO: 54 - protein, 182 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTAS

SEQ ID NO: 55 - protein, 181 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTA

SEQ ID NO: 56 - protein, 180 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMT

SEQ ID NO: 57 - protein, 179 aa, Homo sapiens
DFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 58 - DNA, 33 bp, Artificial Sequence
  Synthetic primer
tctctcgagaaaagagacttccagccggtgctc

SEQ ID NO: 59 - DNA, 537 bp, Homo sapiens
gacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaag

SEQ ID NO: 60 - protein, 178 aa, Homo sapiens
DFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTAS

SEQ ID NO: 61 - protein, 260 aa, Homo sapiens
VYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHERTPENFPCKNLDENYCRNPDGKRAPWCHTTNSQVRWEYCKIPSCDSSPV

SEQ ID NO: 62 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 63 - DNA, 30 bp, Artificial Sequence
  Synthetic primer
atcgtctagagcatccaggcggtggctact

SEQ ID NO: 64 - protein, 92 aa, Homo sapiens
APDTQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 65 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

(19) United States
(12) Patent Application Publication
(10) Pub. No.: US 2003/0012792 A1
(43) Pub. Date: Jan. 16, 2003

(54) Compositions and methods for inhibiting endothelial cell proliferation and regulating angiogenesis using cancer markers

(76) Inventors: John W. Holaday, Bethesda, MD (US); Anne H. Fortier, Germantown, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/131,241

(22) Filed: Apr. 25, 2002

Related U.S. Application Data
(63) Continuation-in-part of application No. 09/907,402, filed on Jul. 17, 2001.
Application No. 09/907,402 is a continuation of application No. 09/413,049, filed on Oct. 6, 1999, now Pat. No. 6,413,513.
Application No. 09/413,049 is a continuation-in-part of application No. 09/316,802, filed on May 21, 1999.
(60) Provisional application No. 60/086,586, filed on May 22, 1998.

(51) Int. Cl. A61K 39/00

(52) U.S. Cl. 424/185.1; 424/277.1

(57) ABSTRACT

Compositions and methods for regulating angiogenesis wherein the compositions comprise cancer markers are provided. Serine proteases and kallikreins exhibit potent antiangiogenic activity on human and other animal cells, particularly endothelial cells. More particularly, the use of a cancer marker, such as PSA, CEA, HCG, NSE, or CA19-9, to inhibit or ameliorate angiogenesis and angiogenesis-related diseases such as cancer, arthritis, macular degeneration, and diabetic retinopathy is disclosed.

### CROSS-REFERENCE TO RELATED PATENT APPLICATIONS

This application claims priority to U.S. patent application Ser. No. 09/413,049, filed Oct. 6, 1999, which claims priority to U.S. application Ser. No. 09/316,802, filed May 21, 1999, which claims priority to U.S. Provisional Application Serial No. 60/086,586, filed May 22, 1998, each of which is incorporated herein by reference in its entirety.

### TECHNICAL FIELD

This invention relates to a novel use of cancer markers for preventing, ameliorating or treating a cell proliferative disease or disorder. The invention further relates to novel compositions and methods for treating angiogenesis-related diseases such as angiogenesis-dependent cancer.

### BACKGROUND OF THE INVENTION

Cancer Markers

Cancer markers are not cancer-specific but rather cancer-associated substances that can be elevated in sera from healthy individuals under various conditions and from patients with benign tumors. The discovery of cancer markers has significantly enhanced not only diagnosis of cancer but has also contributed to the monitoring of cancer patients for assessing disease progression. A rise in cancer markers is a yardstick with which benign diseases can be distinguished from metastatic disease and can also be used to evaluate the efficacy of treatments. A decline in cancer markers is often a predictor of possible residual disease if the timing of blood sampling is soon after therapy.

Numerous cancer markers are known in the art and are utilized in detection assays depending upon the intrinsic characteristics of each marker (antigen specificity, molecular heterogeneity) and individual factors (nonspecific increases and renal and hepatic diseases).

Some reports in literature speculate that lowering cancer markers, such as prostate specific antigen (PSA) production, may inhibit the progression of prostate cancer and therefore a number of initiatives are underway to develop agonists or vaccines against PSA (see, for example, Kim, oncogen 17:3125 (1998) and Hodges Int. J. Cancer 63:231 (1995)).

The inventors of this invention, as disclosed herein, demonstrated that these old strategies should be rethought in view of the potential of cancer markers as antiangiogenic agents that may play a physiological role in restoring homeostasis in the face of progressing angiogenesis and cancer. These observations are therefore consistent with other data indicating that an increase in a cancer marker is correlated with disease cure in certain circumstances (see, for example, Hanlon et al., Cancer, 83:130 (1998)).

Angiogenesis and Cancer

Angiogenesis is a process that is governed by the naturally occurring balance between endogenous stimulators and inhibitors of angiogenesis. Anti-angiogenesis is a process in which inhibitory influences of angiogenesis predominate (Rastinejad et al., Cell 56:345-355 (1989)).

In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases.

A number of serious diseases are dominated by abnormal neovascularization including solid tumor growth and metastases, arthritis, some types of eye diseases, disorders, and/or conditions, and psoriasis (see, i.e., reviews by Moses et al., Biotech. 9:630-634 (1991); Folkman et al., N. Engl. J. Med. 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983)). In a number of pathological conditions, the process of angiogenesis contributes to the disease state. For example, significant data have accumulated which suggest that the growth of solid tumors is dependent on angiogenesis (Folkman and Klagsbrun, Science 235:442-447 (1987)). The process of angiogenesis is complex and involves a number of orchestrated steps that can be separately studied in vitro, such in vitro studies includes, for example, FGF-2- and/or VEGF-stimulated endothelial cell proliferation and migration.

Several lines of direct evidence now suggest that angiogenesis is essential for the growth and persistence of solid tumors and their metastases (Folkman, J., J. Natl. Cancer Inst. 82:4-6 (1989); Hori, et al., Cancer Res. 51:6180-6184 (1991); Kim, et al., Nature 362, 841-844 (1993); Millauer, et al., Nature 367:576-579 (1994)). To stimulate angiogenesis, tumors upregulate their production of a variety of angiogenic factors, including the fibroblast growth factors (FGF and bFGF)(Kandel, et al., Cell 66:1095-1104 (1991)) and vascular endothelial cell growth factor/vascular permeability factor (VEGF/VPF). However, many malignant tumors also generate inhibitors of angiogenesis, including angiostatin and thrombospondin (Chen, et al., Cancer Res. 55:4230-4233 (1995); Good, et al., Proc. Nat. Acad. Sci. USA. 87:6624-6628 (1990); O'Reilly, et al., Cell 79:315-328 (1994)).

It is postulated that the angiogenic phenotype is the result of a net balance between these positive and negative regulators of neovascularization (Good et al., id.; O'Reilly et al. (1994), id.; Parangi, et al., Proc. Natl. Acad. Sci. USA 93:2002-2007 (1996); Rastinejad, et al., Cell 56:345-355 (1989)). Several other endogenous inhibitors of angiogenesis have been identified, although not all are associated with the presence of a tumor. These include, for example, platelet factor 4 (Gupta, et al., Proc. Natl. Acad. Sci. USA 92:7799-7803 (1995); Maione, et al., Science 247:77-79 (1990)), interferon-alpha, interferon-inducible protein 10 (Angiolillo, et al., J. Exp. Med. 182:155-162 (1995); Strieter, et al., Biochem. Biophys. Res. Comm. 210:51-57 (1995)), which is induced by interleukin-12 and/or interferon-gamma (Voest, et al., J. Natl. Cancer Inst. 87:581-586 (1995), gro-beta (Cao, Y., et al., J. Exp. Med. 182:2069-2077 (1995), and the 16 kDa N-terminal fragment of prolactin (Clapp, et al., Endocrinology 133:1292-1299 (1993)).

One example of an angiogenesis inhibitor that specifically inhibits endothelial cell proliferation is angiostatin protein (O'Reilly et al.(1994) id.) Angiostatin protein, is an internal fragment of plasminogen containing about 1 to 5 kringles of plasminogen and has been shown to reduce tumor weight and to inhibit metastasis in certain tumor models. A specific example of angiostatin is ANGIOSTATIN® (O'Reilly et al.(1994) id.) Another example of an angiogenesis inhibitor that specifically inhibits endothelial cell proliferation is endostatin protein (O'Reilly et al., Cell 88:277-285 (1997)). Endostatin protein, includes ENDOSTATINTM that is a carboxy fragment of collagen XV or XVIII, endostatin protein or peptide fragments derived from collagen other than collagen XV or XVIII, endostatin derived from human, mammals, other eukaryotic or prokaryotic tissues and/or organs (see, U.S. Pat. Nos. 5,639,725; 5,854,205; 6,024,688; 5,733,876; and 5,792,845, each of which is incorporated herein by reference in its entirety).

Prostate Specific Antigen

Prostate specific antigen (PSA) is a member of the kallikrein family and an important cancer marker for prostate cancer (Riegman, et al, Genomics 14:6 (1992)). The PSA molecule is a single-chain glycoprotein consisting of approximately 237 amino acids and has a molecular weight of about 28,430 Daltons as determined by ion-spray mass spectroscopy (Sokoll et al., Urologic Clinics of North America, 24:253-259 (1997)). The gene for PSA is located on the long arm of chromosome 19 and is approximately 6 kilobases in size, consisting of 4 introns and 5 exons. The PSA gene is under androgen regulation as evidenced by an androgen-responsive element in the promoter region. PSA is thought to be translated as a 261 amino acid prepropeptide. Although not isolated, the 244 propeptide zymogen form of PSA results after cleavage of the leader peptide during translation. The 237 amino acid active enzyme then is believed to result from subsequent cleavage with as yet unidentified proteases. Structurally, the molecule is thought to possess five disulfide bonds due to the presence of 10 cysteine residues with the active site of the enzyme composed of three amino acids, histidine 41, aspartate 96 and serine 189.

PSA is synthesized in the ductal epithelium and prostatic acini and located within the cell in cytoplasmic granules and vesicles, rough endoplasmic reticulum, vacuoles and secretory granules, and lysosmal dense bodies. PSA is found in normal hyperplastic, primary, and metatstatic prostate tissue. PSA is secreted into the lumina of the prostatic ducts via exocytosis to become a component of seminal plasma and reaches serum after diffusion from luminal cells through the epithelial basement membrane and prostatic stroma, where it can pass through the capillary basement membrane and epithelial cells or into the lymphatics (Sokoll et al. id).

Despite original assumptions that PSA was a tissue-specific and gender-specific antigen, immunohistochemical and immunoassay methods have detected PSA in female and male periurethral glands, anal glands, apocrine sweat glands, apocrine breast cancers, salivary gland neoplasms, and most recently in human breast milk.

PSA functions as a serine protease exhibiting proteolytic activity similar to chymotrypsin, cleaving peptide bonds carboxy terminus of certain leucine and tyrosine residues. Based on its function, amino acid structure and gene location, PSA is recognized as a member of the human kallikrein family.

In males, PSA is secreted from the lumen of the prostate and enters the seminal fluid as it passes through the prostate. In the seminal fluid are gel-forming proteins, primarily semenogelin I and II and fibronectin, which are produced in the seminal vesicles. These proteins are the major constituents of the seminal coagulum that forms at ejaculation and functions to entrap spermatozoa. PSA functions to liquefy the coagulum and break down the seminal clot through proteolysis of the gel-forming proteins into smaller more soluble fragments, thus releasing the spermatozoa. PSA may also modulate cell growth factor (IGF) binding protein 3, resulting in decreased binding with IGF-1, thus promoting cell growth (Sokoll et al. id.)

Kallikrein

Kallikrein and kallikrein-like enzymes belong to a multigene family of serine proteases present in tissues and body fluids of numerous animals such as mammals and reptiles (i.e. snake venom). Included in the kallikrein family is hk1, a pancreatic/renal kallikrein; hk2, a human glandular kallikrein present in seminal fluid, a protease that activates urokinase type plasminogen activator; and prostate-specific antigen (hk3), a single-chain glycoprotein found in prostate tissue. Pre-kallikrein is converted by limited proteolysis into an active serine protease, and is one of the five major proteins involved in the activation and inhibition of surface mediated pathways in blood clotting. Pre-kallikrein is an important component of the biochemical junctures of intrinsic coagulation with other plasma proteolytic pathways required in the initiation, amplification, and propagation of surface-mediated defense reactions wherein various proteins such as bradykinin are involved. Thus, the molecular events of the contact phase of coagulation activation and inhibition involve pre-kallikrein and the plasma biochemical systems (Colman et al., Curr. Top. MicrobiolImmunol. 231:125 (1998)).

Plasma kallikrein circulates in the blood as the precursor “pre-kallikrein.” Plasma pre-kallikrein is synthesized in the liver and secreted into plasma. However, only 25% of the protein exists as free pre-kallikrein and approximately 75% circulates bound to high molecular weight kininogen (HMWK). The molecular weight of human plasma pre-kallikrein, as assessed by gel filtration, is approximately 100,000 Daltons. By SDS polyacrylamide gel electrophoresis, plasma pre-kallikrein consists of two components having molecular weight 85,000 Daltons and 88,000 Daltons, depending whether the sample has undergone reduction. In plasma, the concentration of pre-kallikrein is estimated to be 35 μg to 50 μg/ml.

Following proteolysis, pre-kallikrein is activated to kallikrein. Current studies do not demonstrate any clear cut difference in physiochemical or immunochemical properties of zymogen pre-kallikrein, and active enzyme kallikrein in the absence of reduction. Hageman factor (also known as Factor XIIa) and Hageman factor fragment (also known as Factor XIIf) are both able to convert pre-kallikrein to kallikrein. Unlike pre-kallikrein on reduced SDS gel electrophoresis, kallikrein has two types of subunits: A heavy chain with a molecular weight of approximately 52,000 Daltons, and two light chain variants with a molecular weight of approximately 36,000 Daltons and 33,000 Daltons. Pre-kallikrein circulates mostly complexed to high molecular weight kininogen HMWK, and it is thought that this complex may have protective functions for the pre-kallikrein. Following activation from pre-kallikrein to kallikrein, HMWK is cleaved to release bradykinin. Bradykinin is one of the most potent vasodilators known (Colman et al. id.)

The gene for plasma pre-kallikrein has not been isolated or characterized thus far. The messenger RNA for plasma pre-kallikrein, however has been characterized and shown to be approximately 2,300 nucleotides in length. The cDNA of pre-kallikrein codes for a leader sequence of 19 amino acids and a mature polypeptide chain of 619 amino acids. The latter peptide in plasma pre-kallikrein is one amino acid longer than that in Factor XI. The activation reaction of pre-kallikrein to kallikrein is due to the cleavage of the peptide bond following arginine 371. Plasma kallikrein is generated as an enzyme composed of a heavy chain (371 amino acids) and a light chain (248 amino acids), held together by a disulfide bond. The catalytic domain or light chain of plasma kallikrein, contains three important amino acids (His-44, Asp-93 and Ser-188) that are directly involved in catalysis. In addition, plasma kallikrein contains 5 N-linked carbohydrate chains as established by amino acid sequence analysis.

The proteins and enzymes of the clotting cascade may perform multiple functions, for example, Factor XIIₐ may cleave pre-kallikrein to kallikrein, and Factor XI to XIₐ. Kallikrein can initiate reciprocal activation, generating additional Factor XIIₐ from Factor XII. Plasma kallikrein leads to the conversion of plasminogen to plasmin and Factor XIIₐ also converts plasminogen to plasmin. Kallikrein cleavage of HMWK results in the release bradykinin and may also elevate blood pressure by directly converting pro-renin to renin.

Patients with bacterial infections, especially those caused by gram negative bacteria, may have elevated levels of plasma kallikrein. The hypotensive effect of kallikrein may contribute to the development of disseminated intravascular coagulation by reducing blood flow to reticuloendothelial organs thereby impairing clearance of activated coagulation factors.

Despite the extensive characterization of certain cancer markers to date, there is no report in literature concerning their antiangiogenic potential. What is needed is the discovery and development of cancer markers with angiogenic regulatory activity that may be used alone or in combination with known angiogenic inhibitors in order to treat cancer and hyperproliferative disorders. This invention satisfies these and other needs.

### SUMMARY OF THE INVENTION

This invention is directed to novel methods and compositions for treating angiogenesis-related diseases such as angiogenesis-dependent cancer. The method described in the present application recognizes a body's attempt to restore homeostasis during a pathologic-angiogenic assault and employs this concept to provide a new composition and method against pathologic angiogenesis. In particular, this invention generally relates to the identification and use of antiangiogenic cancer markers for treating angiogenic related diseases.

Accordingly, in one aspect of the present invention compositions and methods are provided for treating, ameliorating, or preventing cell proliferative diseases or disorders, such as undesired or uncontrolled angiogenesis, by administering to a human or animal with the undesired angiogenesis, or at risk of contracting undesired angiogenesis, a composition comprising a cancer marker in a dosage sufficient to inhibit or prevent angiogenesis.

A particularly important aspect of the present invention is the discovery of a novel and effective method for treating angiogenesis-related diseases, by the administration of a cancer marker, or the co-administration of a cancer marker and another anti-angiogenesis compound, such as endostatin (including ENDOSTATIN™ protein, or a peptide fragment thereof) and/or angiostatin (including ANGIOSTATIN® protein, or a peptide fragment thereof).

According to one embodiment of the invention, the cancer marker includes prostate specific antigen (PSA), carcinoembryonic antigen (CEA), neuron specific enolase (NSE), human chorionic gonadotropin (HCG-α, HCG-β), cancer antigen (CA 19-9), analogs, derivatives, variants, substantially homologous peptides, mimetics, agonists, antagonists, or fusion peptides of these cancer markers. In a preferred embodiment of the invention, the cancer marker is administered with an angiogenic inhibitory peptide, a cytotoxic drug or both.

According to one aspect of the invention, a pharmaceutical composition containing a cancer marker, is used to inhibit or ameliorate the growth of benign tumors, neovascular diseases of the eye and increases apoptosis.

According to another aspect of the invention, pharmaceutical compositions and methods are provided for treating or repressing the growth of a cancer. In one embodiment, the invention provides a method for the specific destruction of cells (i.e., the destruction of tumor cells) by administering the cancer marker of the invention in association with toxins or cytotoxic prodrugs.

It is another aspect of the invention to provide compositions and methods for the detection or prognosis of angiogenesis activity.

It is yet another aspect of the present invention to provide pharmaceutical compositions that are utilized in a wide variety of surgical procedures. For example, in one embodiment of the present invention, a composition is utilized to coat or spray an area prior to removal of a tumor, in order to isolate normal surrounding tissues from malignant tissue, and/or to prevent the spread of disease to surrounding tissues. In other embodiments of the present invention, compositions are delivered via endoscopic procedures in order to coat tumors, or inhibit angiogenesis in a desired locale. In yet other embodiments of the present invention, surgical meshes which have been coated with antiangiogenic compositions of the present invention may be utilized during abdominal cancer resection surgery (i.e., subsequent to colon resection) in order to provide support to the structure, and to release an amount of the antiangiogenic factor.

According to another aspect of the present invention, methods are provided for treating tumor excision sites, comprising administering the pharmaceutical composition to the resection margins of a tumor subsequent to excision, such that the local recurrence of cancer and the formation of new blood vessels at the site is inhibited. In one embodiment of the invention, the pharmaceutical composition is administered directly to the tumor excision site (i.e., applied by swabbing, brushing or otherwise coating the resection margins of the tumor with the antiangiogenic compound).

In yet another aspect of the present invention, a new form of birth control is provided, wherein a pharmaceutically effective amount of a cancer marker of the invention, such as kallikrein (for example, PSA), is administered to a patient in need thereof such that uterine endometrial vascularization is inhibited and embryo implantation cannot occur or be sustained.

It is yet another aspect of the present invention to provide a therapy for cancer that has minimal side effects.

It is another aspect of the present invention to provide a method of screening a compound for its ability to regulate angiogenesis comprising: (a) identifying a candidate cancer marker, (b) preparing the cancer marker for testing and (c) testing the cancer marker in at least one bioassay to determine an inhibitory affect of the cancer marker on endothelial cell proliferation and/or formation, wherein an inhibitory effect of the cancer marker in at least one bioassay correlates with an angiogenesis inhibitory activity of the cancer marker. The testing is performed by one or more in vitro, in vivo, ex vivo, or in situ bioassays. Bioassays, include for example and not by way of limitation, a proliferation assay, migration assay, invasion assay, cord formation assay, or apoptosis assay. In one embodiment, bioassays include, human umbilical vein endothelial cell proliferation assay (HUVEC), the bovine capillary endothelial cell proliferation assay (BCE), chick CAM assay, mouse corneal assay, matrigel assay, implanted tumor assay, or a combination thereof.

It is yet another aspect of the present invention to provide drug screening methods for identification and isolation of drugs that modify the angiogenic regulatory ability of cancer markers. Such a drug screening method includes, for example, contacting cancer marker of the present invention with a selected compound(s) suspected of having antagonist or agonist activity, and assaying the activity of these polypeptides following binding.

In one embodiment, the screening method is used for identifying polypeptides, nucleic acids and/or small molecules that bind a polypeptide-based cancer marker of the invention. These binding molecules are useful, for example, as agonists and antagonists of the cancer marker of the invention. Such agonists and antagonists can be used, in accordance with the invention, in the therapeutic embodiments described in detail below.

These and other aspects, features and advantages of the present invention will become apparent after a review of the following detailed description of the disclosed embodiments and the appended claims.

### BRIEF DESCRIPTION OF THE FIGURES

FIG. 1. Effect of native PSA on angiogenesis in a Matrigel Plug. Matrigel supplemented with 2 mg/ml of FGF-2 was injected subcutaneously under the skin of male C57BL6/J mice. Animals were treated subcutaneously with native PSA (100 μg/day) or Endostatin™ protein (5 mg/kg/day). The concentration of hemoglobin was measured as an assessment of angiogenic responsiveness.

FIG. 2. A dose response graph demonstrating the inhibition of proliferation activity in bFGF-stimulated human umbilical vein endothelial cells following administration of PSA.

FIG. 3. A graph demonstrating the inhibition of proliferation activity in bFGF-stimulated bovine capillary endothelial cells following administration of PSA.

FIG. 4. A graph demonstrating the effect of PSA on proliferation of human umbilical vein endothelial cells (HUVEC) in vitro.

FIG. 5. A graph demonstrating the effect of PSA on proliferation of bovine capillary endothelial cells (BCE) in vitro.

FIG. 6. A graph demonstrating the effect of PSA on proliferation of human microvascular dermal cells (HMVEC-d) in vitro.

FIG. 7. A graph demonstrating the effect of PSA on proliferation of murine melanoma B16BL6 cells (tumor cell lines).

FIG. 8. A graph demonstrating the effect of PSA on proliferation of human prostate carcinoma (PC3).

FIG. 9. A graph demonstrating the effect of PSA on migration of FGF-2-stimulated HUVECs.

FIG. 10. A graph demonstrating the effect of PSA on migration of VEGF-stimulated HUVECs.

FIG. 11. A graph demonstrating the proteolytic activity of PSA using the synthetic substrate S-2586 (MeO-Suc-Arg-Pro-Tyr-NH-Np). The results are plotted as an increase in absorbance vs. time in minutes. PSA (0.89 μM) (square) or ACT (0.92 μM)(circle) were incubated alone with substrate and hydrolysis measured over 40 min. For analysis of an inhibitory effect of ACT on PSA: PSA was preincubated with (inverted triangle) or without (regular triangle) equimolar amounts of ACT at 37° C. for 4 h prior to the addition of substrate. Upon addition of substrate, hydrolysis was measured over 40 min.

FIG. 12. A graph demonstrating HUVEC migration inhibitory activity of PSA as assessed in the presence or absence of ACT. For comparison, the number of cells that migrated in response to media alone and FGF-2 is shown. Active PSA was preincubated with an equimolar concentration of ACT.

FIG. 13. A graph demonstrating the effect of native PSA and recombinant PSAs (intact and N−1 variant) on VEGF-induced migration of HUVEC. Native PSA (FIG. 13A), as well as recombinant PSA (FIG. 13B), and N−1 recombinant PSA (FIG. 13C), inhibit angiogenesis by at least 50%. Each data point is the mean of observations from quadruplicate cultures ±1 standard deviation. Results are representative of three similar experiments.

FIG. 14. A graph demonstrating HUVEC migration microhemotaxis assay with rCEA. rCEA inhibited VEGF-induced migration of HUVEC with an IC₅₀ between 10 and 100 ng/ml. Higher concentrations from 1-100 μg/ml were stimulatory.

FIG. 15. A graph demonstrating HUVEC cord formation on matrigel treated with rCEA. In addition, concentration of 1 ng/ml of rCEA inhibited HUVEC cord formation on matrigel by 28% while higher concentrations of 1-100 μg/ml enhanced cord formation.

FIG. 16. A graph demonstrating CA 19-9 at an IC₅₀ of 1000 U/ml inhibited FGF-2-stimulated HUVEC proliferation. The addition of 10,000 U/ml blocked cord formation by 38%. Concentrations of 1000 U/ml inhibited cord formation by 25%. No significant differences were observed with doses of 100 U/ml or less.

FIG. 17. A graph demonstrating HUVEC migration microchemotaxis assay treated with CA 19-9.

FIG. 18. A graph demonstrating HUVEC cord formation on matrigel treated with CA 19-9.

FIG. 19. A graph demonstrating HCG-α subunit inhibited FGF-2 HUVEC proliferation with an IC₅₀ between 10 and 100 μg/ml. The concentration of HCG-A that resulted in 50% inhibition of VEGF stimulated HUVEC migration was 100 μg/ml.

FIG. 20. A graph demonstrating that HCG-β was able to induce apoptosis of HUVEC in a dose-dependent fashion. A 13% induction of apoptosis was observed with a concentration of 10 μg/ml while 4% was observed after treatment with 1 μg/ml.

FIG. 21. A graph demonstrating HUVEC migration microchemotaxis assay treated with HCG-P.

FIG. 22. A graph demonstrating HUVEC cord formation on matrigel of HCG-β.

FIG. 23. A graph demonstrating HUVEC cord formation index of HCG-α and HCG-β. Media control (p=0.02).

FIG. 24. A graph demonstrating HUVEC BrdU Elisa assay of both HCG-α and HCG-β.

FIG. 25. A graph demonstrating HUVEC BrdU Elisa proliferation assay of NSE. NSE inhibited FGF-2-stimulated HUVEC proliferation with an IC₅₀ of 20 μg/ml (concentration at which inhibition was 50%).

FIG. 26. Nucleic acid and amino acid sequences of rCEA.

### DETAILED DESCRIPTION OF THE INVENTION

Applicants have discovered a novel property for a class of biomolecules. As a result of their investigations, the inventors of the present invention have surprisingly demonstrated for the first time that several biomolecules in the class of cancer markers are potent regulators of angiogenesis. These cancer markers are generally known to be useful in the diagnosis and prognosis of cancer. The regulatory affect on angiogenesis is demonstrated, for example, by potent anti-proliferative and/or anti-migratory activity of cancer markers on a variety of cultured endothelial cells, thus exhibiting an endothelial cell-specific inhibition of angiogenesis. Based on the novel findings of the inventors, the present invention is directed to methods and compositions comprising the administration of these biomolecules for the regulation of angiogenesis.

Definitions

As used herein “cancer markers” refer to molecules of diverse structure and function that are under-expressed, over-expressed, or aberrantly-expressed in an individual having a cancer or exposed to the risk of developing cancer. Cancer markers may be polypeptide or nucleotide-based molecules or non-polypeptide or nucleotide-based molecules, including small molecules, carbohydrates, lipids, or a combination thereof.

As used herein “polypeptide-based cancer markers” include any protein, polypeptide or peptide fragment that is produced in the course of the transcription, reverse-transcription, polymerization, translation, post-translation and/or expression of a nucleotide molecule. The polypeptide-based cancer marker of the invention includes, for example, biologically active fragments, substantially homologous polypeptides, oligopeptide, homodimers, heterodimers, variants of the polypeptides, modified polypeptides, derivatives, analogs, fusion proteins, agonists, antagonists, or antibody of the polypeptide, among others. The polypeptides of the invention are natural peptides, recombinant peptides, synthetic peptides, or a combination thereof.

As used herein “biologically active fragments” refer to fragments exhibiting activity similar, but not necessarily identical, to an activity of the cancer marker or the antiangiogenic peptide of the present invention. The biologically active fragments may have improved desired activity, or a decreased undesirable activity.

As used herein “polypeptides” include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

As used herein “nucleotide-based cancer markers” include cDNA, RNA, DNA/RNA hybrid, anti-sense RNA, mRNA, ribozyme, and genomic DNA, among others.

As used herein “antiangiogenic polypeptides” include, but are not limited to, angiostatin (i.e., ANGIOSTATIN®), endostatin (i.e., ENDOSTATIN™), metastatin (i.e., METASTATIN™) HGF, TFPI, anti-invasive factors, retinoic acid and derivatives thereof, paclitaxel, suramin, tissue inhibitor of metalloproteinase-1, tissue inhibitor of metalloproteinase-2, plasminogen activator inhibitor-1, plasminogen activator inhibitor-2, among others.

As used herein “angiostatin” includes ANGIOSTATIN®, which is a recombinant human angiostatin, angiostatin peptide fragments containing kringle fragments 1-5, or any combinations of kringles 1-5 such as, kringles 1, 2, 3, 4, 5, 1-2,1-3, 1-4,1-5, 2-3,2-4, 2-5,3-4, 3-5,4-5, biologically active fragments of angiostatin that elicit a biological activity either in vitro or in vivo, substantially homologous peptides, oligopeptide, homodimers, heterodimers, variants of the peptides, modified peptides, or fusion proteins, or a combination thereof (see, U.S. Pat. Nos. 5,639,725; 5,854,205; 6,024,688; 5,733,876; and 5,792,845, each of which is incorporated herein by reference in its entirety).

As used herein “endostatin” includes ENDOSTATIN™, which is a carboxy fragment of collagen XV or XVIII, endostatin protein or peptide fragments derived from carboxy fragment of collagens other than collagen XV or XVIII, endostatin derived from non-collagen precursor proteins, endostatin derived from human, mammals, other eukaryotic or prokaryotic tissues and/or organs, biologically active fragments that elicit a biological activity either in vitro or in vivo, substantially homologous peptides, oligopeptide, homodimers, heterodimers, variants of the peptides, modified peptides, or fusion proteins (see, U.S. Pat. Nos. 5,854,205 and 6,346,510, each of which is incorporated herein by reference in its entirety).

As used herein “small molecules” include, but are not limited to, carbohydrates, carbohydratemimetics, peptidomimetics, organic or inorganic compounds (i.e, including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein “pharmaceutical composition” refers to a composition that contains a cancer marker and a pharmaceutically acceptable carrier or diluent. The “pharmaceutical composition” also refers to a composition that additionally contains an antiangiogenic polypeptide and/or cytotoxic agents. The “pharmaceutical composition” also refers to a composition that contains a cancer marker agonist, antagonist, biologically active fragments, variants, analogs, modified peptides, substantially homologous sequences thereof.

As used herein “fusion protein” refers to a protein encoded by two or more, often unrelated, fused genes or fragments thereof. Membrane bound proteins, such as protein disulfide isomerase (PSI) are particularly useful in the formation of fusion proteins. Such proteins are generally characterized as possessing three distinct structural regions, an extracellular domain, a transmembrane domain, and a cytoplasmic domain. This invention contemplates the use of one or more of these regions as components of a fusion protein.

As used herein “cell proliferative disease” refers to any human or animal disease or disorder, affecting any one or any combination of organs, cavities, or body parts, which is characterized by single or multiple local abnormal proliferations of cells, groups of cells, or tissues, whether benign or malignant.

As used herein, the term “angiogenesis” and related terms such as “angiogenic” refer to activities associated with blood vessel formation, growth and development, including, but not limited to, endothelial cell proliferation, endothelial cell migration and capillary tube formation, among others.

As used herein, the term “antiangiogenic” refers to compositions and the like that are capable of inhibiting or reducing the formation or growth of blood vessels, including but not limited to inhibiting or reducing endothelial cell proliferation, endothelial cell migration and capillary tube formation, among others.

Cancer Markers

Cancer markers of the invention include, but are not limited to, prostate specific antigen (PSA), human chorionic gonadotropin (HCG-α and HCG-β, cancer antigen 19-9 (CA 19-9), carcinoembryonic antigen (CEA), neuron specific enolase (NSE), squamous cell carcinoma-associated antigen (SCC), alpha-fetoprotein, cancer antigen (CA)125, CA15-3, CD20, CDH13, CD31, CD34, CD 105, CD 146, D16S422HER-2, phospatidylinositol 3-kinase (PI 3-kinase), trypsin, trypsin-1 complexed with alpha(1)-antitrypsin, estrogen receptor, progesterone receptor, c-erbB-2, bcl-2, S-phase fraction (SPF), p185erbB-2, low-affinity insulin like growth factor-binding protein, urinary tissue factor, vascular endothelial growth factor, epidermal growth factor, epidermal growth factor receptor, apoptosis proteins (p53, Ki 67), factor VIII, adhesion proteins (CD-44, sialyl-TN, blood group A), bacterial LacZ, human placental alkaline phosphatase (ALP), alpha-difluoromethylomithine (DFMO), thymidine phosphorylase (dTHdPase), thrombomodulin, laminin receptor, fibronectin, anticyclins, anticyclin A, B, or E, proliferation associated nuclear antigen, lectin UEA-1, von Willebrand's factor, or a combination thereof.

According to one embodiment, the cancer marker comprises, PSA, HCG-α, HCG-β, CA 19-9, CEA, and NSE.

As a result of their investigations, the inventors of the present invention have surprisingly demonstrated for the first time that cancer markers, such as PSA, are an endothelial cell-specific inhibitor of angiogenesis and exhibit potent anti-proliferative and anti-migratory activity on a variety of cultured endothelial cells. Furthermore, PSA inhibits the endothelial-cell specific angiogenesis process of capillary tube formation in a matrigel assay.

Based on the novel findings of the inventors, the present invention is directed to methods and compositions comprising the administration of cancer markers such as PSA and other members of the serine protease family, including kallikreins, for the regulation of antiangiogenic processes. More particularly, the methods and compositions of the present invention comprise the administration of PSA for inhibiting angiogenesis and for reducing related cancer or tumor growth.

“Prostate-Specific Antigen” (PSA) is a protein belonging to the family of kallikreins and as used herein, it is to be understood that the term PSA includes PSA analogs, homologs and active peptides thereof. As it is used hereinafter, the term “PSA” refers to PSA as described above, peptide fragments of PSA that have angiogenesis inhibiting activity, and analogs of PSA that have substantial sequence homology (as defined herein) to the amino acid sequence of PSA, which have angiogenesis inhibiting activity. The term “kallikrein” refers to a family of serine proteases found in tissues and body fluids of numerous animals including mammals and reptiles. The family of kallikreins includes enzymes such as hk1, a pancreatic renal kallikrein, human glandular kallikrein (hk2), and prostate-specific antigen (hk3). Plasma kallikrein usually circulates in the blood as pre-kininogen (HMWK). Following proteolysis, pre-kallikrein is activated to kallikrein which then cleaves HMWK to release bradykinin. The kallikreins, HMWK, and bradykinin represent some of the important proteins involved in the activation and inhibition of surface mediated pathways involved in blood clotting. As used herein, the term “kallikrein” refers to kallikrein analogs, homologs and active peptides thereof having the ability to regulate angiogenic activity.

Prostate-Specific Antigen (PSA) refers generally to a protein that is approximately 26,000-32,000 Daltons in size as determined by ion-spray mass spectroscopy, more specifically to a protein that is 28,000-29,000 Daltons, and more preferably to a protein that is 28,430 Daltons. The amino acid sequence of a human PSA is provided in SEQ ID NO: 62. The term PSA also includes precursor forms of the prepropeptide and propeptide as well as modified proteins and peptides that have a substantially similar amino acid sequence, and which are capable of inhibiting proliferation of endothelial cells. For example, silent substitutions of amino acids, wherein the replacement of an amino acid with a structurally or chemically similar amino acid does not significantly alter the structure, conformation or activity of the protein, are well known in the art. Such silent substitutions, additions and deletions, are intended to fall within the scope of the appended claims.

It will be appreciated that the term “PSA” includes shortened proteins or peptides wherein one or more amino acid is removed from either or both ends of PSA, or from an internal region of the protein, yet the resulting molecule retains angiogenic regulating activity. The term “PSA” also includes lengthened proteins or peptides wherein one or more amino acid is added to either or both ends of PSA, or to an internal location in the protein, yet the resulting molecule retains angiogenic regulating activity. Such molecules, for example with tyrosine added in the first position, are useful for labeling such as radioiodination with ¹²⁵Iodine, for use in assays. Labeling with other radioisotopes may be useful in providing a molecular tool for isolating and identifying the target cell containing PSA receptors. Other labeling, with molecules such as ricin, may provide a mechanism for destroying cells with PSA receptors. The invention also contemplates that active peptides of PSA may be used alone or combined with other peptides and proteins to form chimeric proteins containing the active PSA peptide.

PSA can be isolated from normal, hyperplastic, primary and metatstatic prostate tissue from a variety of species including humans. PSA can also be isolated from body fluids including, but not limited to, semen, serum, urine and ascites, or synthesized by chemical or biological methods (i.e. cell culture, recombinant gene expression, peptide synthesis and in vitro enzymatic catalysis of precursor molecules to yield active PSA). In addition, PSA may be produced from recombinant sources, from genetically altered cells implanted into animals, from tumors, and from cell cultures as well as other sources. Recombinant techniques include gene amplification from DNA sources using the polymerase chain reaction (PCR), and gene amplification from RNA sources using reverse transcriptase/PCR.

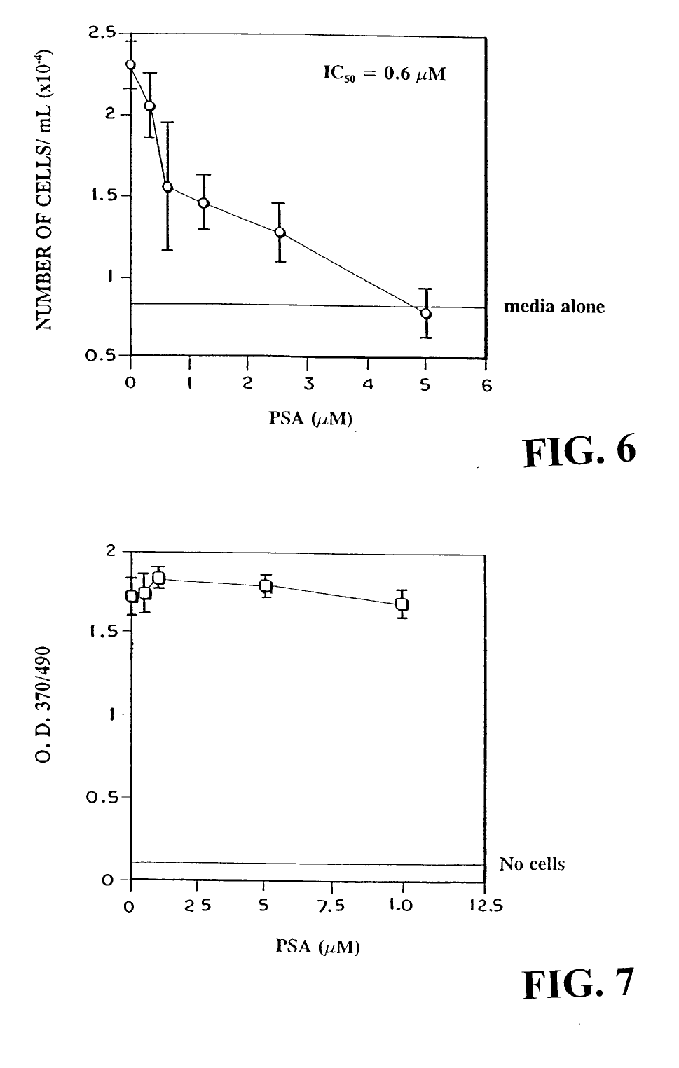

The inventors of the present invention have surprisingly discovered antiangiogenic properties of kallikreins, such as PSA. As explained in more detail in the Examples below, the effects of PSA on angiogenic activity were first shown in Human Umbilical Vein Endothelial Cells (HUVEC). Purified human PSA demonstrated a potent and dose related inhibitory activity on FGF-2-stimulated proliferation of HUVEC cells. To determine if PSA inhibited a variety of endothelial cells or simply displayed specificity for HUVECs, the ability of PSA to inhibit bovine adrenal cortex endothelial cell (BCE) and human microvascular dermal cell (HMVEC-d) proliferation was also evaluated. It was discovered that PSA potently inhibited FGF-2-stimulated endothelial cell proliferation, with an IC₅₀ for BCE cells of 1.0 μM, and an IC₅₀ for HMVEC-d of 0.6 μM (see, FIGS. 5 and 6, respectively).

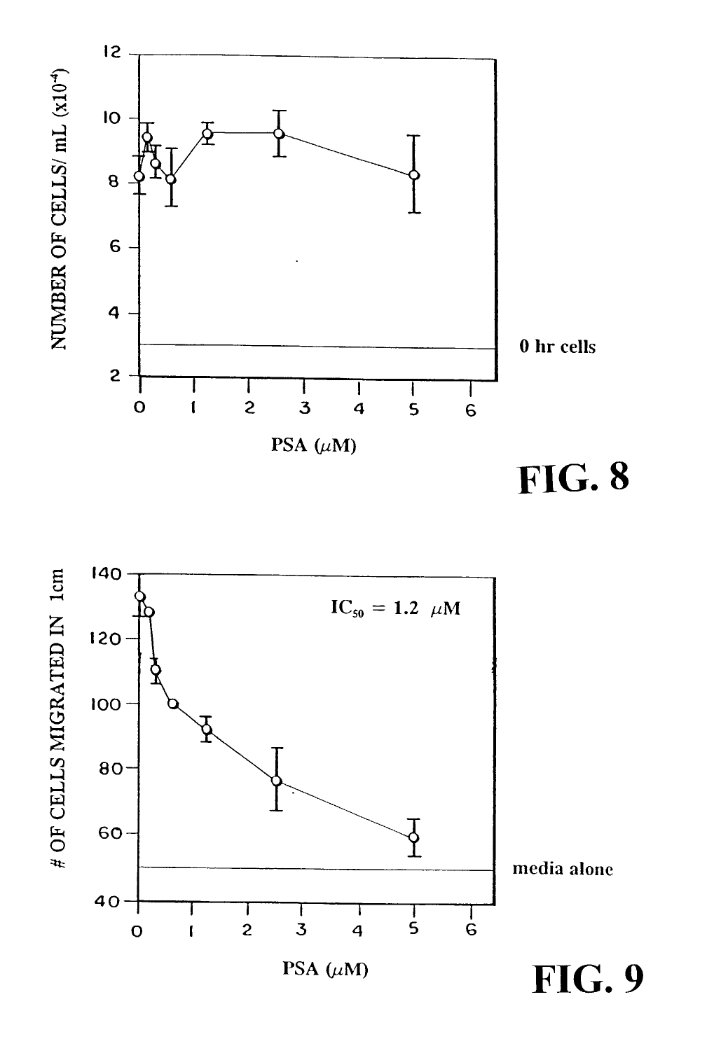

In order to demonstrate that PSA exerts antiangiogenic effects as opposed to general inhibition of cell proliferation, the inventors conducted experiments to evaluate direct stimulatory or inhibitory effect on the proliferation of cancer cells. As disclosed in Example 6, the growth of murine melanoma cells (B16BL6) or human prostate cancer cells (PC3) was unaffected by the addition of purified human PSA (see, FIGS. 7 and 8, respectively) thereby confirming PSA antiangiogenic activity.

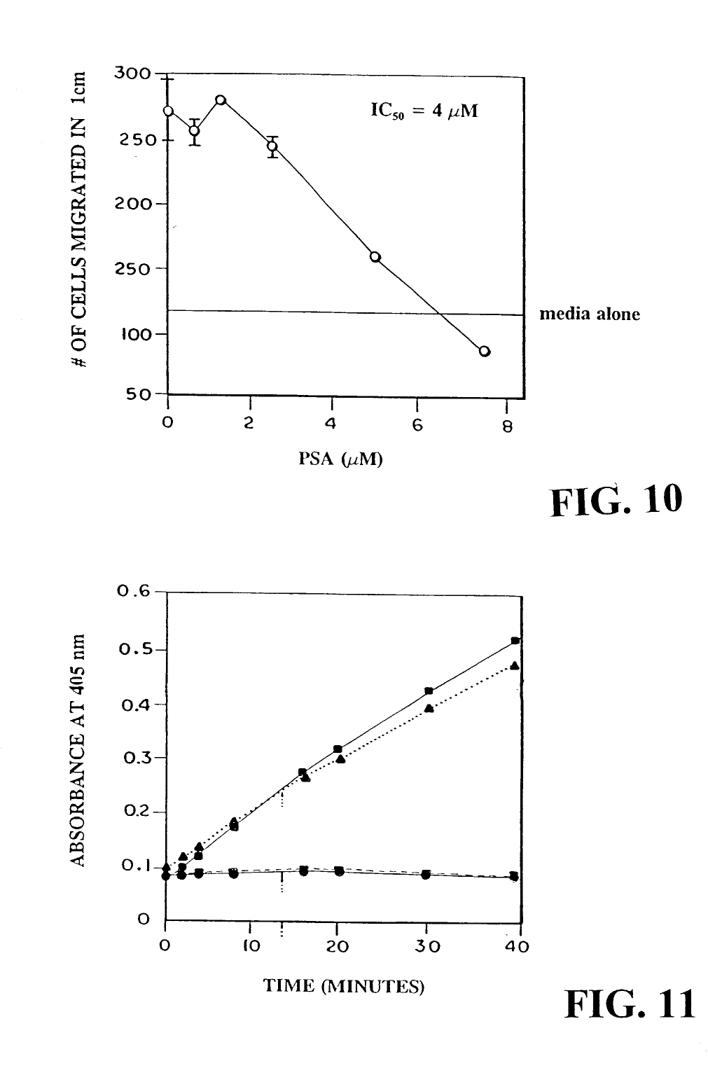

The effects of PSA on endothelial cell migration were demonstrated by the inventors to further confirm the antiangiogenic effects of PSA. In order to evaluate the in vitro effects of PSA on endothelial cell migration in response to FGF-2 or VEGF, confluent monolayers of HUVEC were scraped to remove a section of monolayer and cultured with FGF-2 or VEGF in the presence or absence of purified human PSA (see, Example 7). As shown in the figures, PSA exerted dose-response inhibitory effects on FGF-2 and VEGF-stimulated migration (see, FIGS. 9 and 10, respectively).

The inventors further demonstrated antiangiogenic properties of PSA by evaluating its effects on endothelial cell invasion. As further discussed in the examples below, the results of these experiments demonstrated that inhibition appeared to be dose dependent and not the result of toxicity since the endothelial cells appeared viable and, although some elongation was noted, there were no junctions made by the endothelial cells. These findings demonstrate the inhibitory effects of PSA on endothelial cell invasion and further confirm PSA antiangiogenic activity.

Though not wishing to be bound by the following theory, it is believed that the antiangiogenic properties of PSA are related to its serine protease activity. As demonstrated in Example 9, when the serine protease activity of PSA was blocked, the anti-proliferative and anti-migratory effects of PSA on endothelial cell were also inhibited.

The antiangiogenic serine proteases of the present invention can be generated by automated protein synthesis methodologies well-known to one skilled in the art. Alternatively, antiangiogenic serine proteases, or kallikreins, including PSA and peptide fragments thereof, may be isolated from larger known pre-propeptides that share a common or similar amino acid sequence.

According to one embodiment of the invention, carcinoembryonic antigen (CEA) is used as a regulator of angiogenesis. CEA is a surface glycoprotein with molecular weight of approximately 180 kD. CEA is an oncodevelopmental human tumor marker that normally occurs on basolateral cell membranes in embryonic intestine (fetal gut tissue), and disappears after birth. CEA is also found in very low amounts on lumenal aspect of epithelial cells in normal adults. This cancer marker is used to detect colon cancers (adenocarcinomas of the human digestive system).

The role of the CEA immunoassay for diagnosis and serially monitoring cancer patients for recurrent disease or response to therapy, particularly in the case of colonic cancer, have been widely evaluated and documented. But in practicing the immunoassay for such purposes, one encounters difficulties because of the presence of CEA-like substances in normal colonic mucosa and in the serum, saliva, faeces and colon lavages of apparently normal individuals. These CEA-like substances share common and distinct binding sites with CEA suggesting the presence of very closely related genes as well as precursor-product relationships between some of these genes (see, for example, Rogers, Biochim, Biophys. Acta, 695: 227-249 (1981)). It is essential, therefore, to clarify the molecular nature of these antigens for establishing the tumor-specific assays for clinical use and for the study of their biological significance.

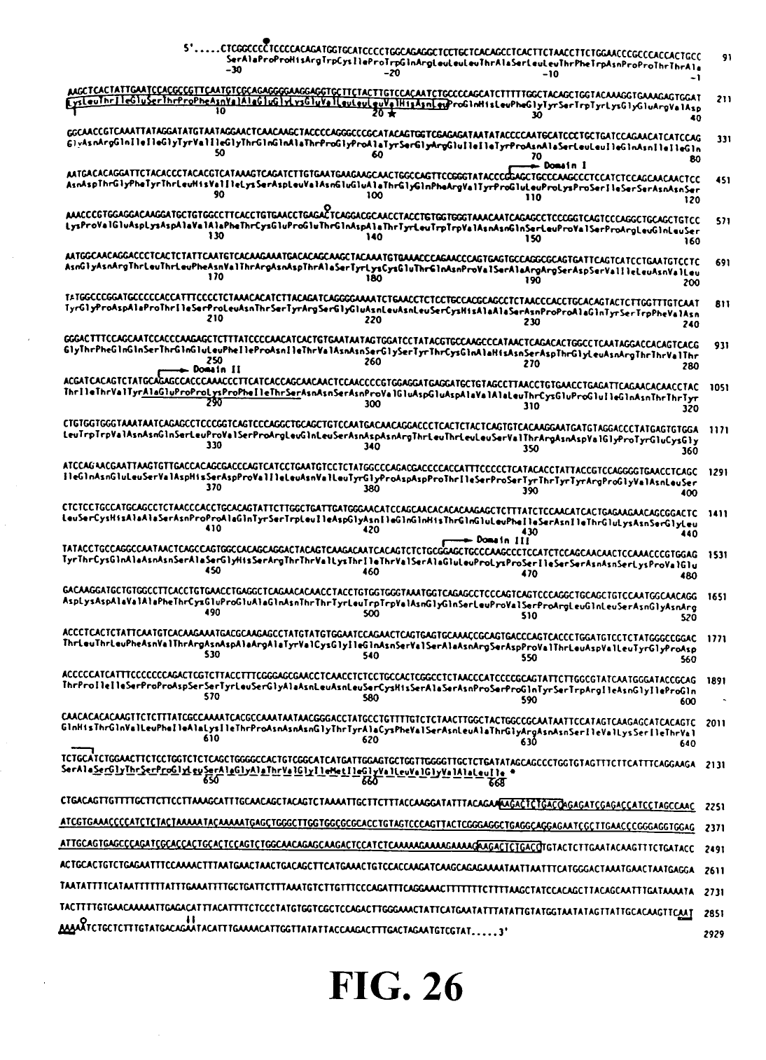

Cloning of the cDNA corresponding to the mRNA encoding a polypeptide which is immunoreactive with the antisera specific to CEA and the primary structure of the precursor for the putative CEA deduced from the nucleotide sequences of the cDNAs are disclosed in Shinzo et al., Biochemical and Biophysical Res. Commu. 142: 511-518 (1987), content of which is incorporated herein by reference in its entirety)). The nucleic acid and the amino acid sequences of rCEA is disclosed herein as FIG. 26.

The antiangiogenic effect of CEA is demonstrated for the first time by the inventors of the present invention. The results, as disclosed and described in the examples below, demonstrate that both CEA and recombinant CEA (rCEA) demonstrated antiangiogenic activity.

According to another embodiment of the invention, cancer antigen (CA 19-9) is used as a regulator of angiogenesis. CA19-9 refers to a large cancer antigen found on cancers of the pancreas, the stomach, the bile and to a lesser extent cancer of the colon. CA 19-9 is produced by adenocarcinomas of the pancreas, stomach, gall bladder, colon, ovary, and lung, and it is shed into the circulation. High values of CA19-9 have also been found in patients with lung cancer. CA 19-9 assay measures a tumor related mucin that contains the sialylated Lewis-a pentasaccharide epitope, lacto-N-fucopentaose II. The results of the experiments described herein demonstrate that CA19-9 has an angiogenic inhibiting activity.

According to yet another embodiment of the invention, human chorionic gonadotrophin (HCG) is used as a regulator of angiogenesis. Glycoprotein hormones (or gonadotropins) are a family of proteins which include the mammalian hormones follitropin (FSH), lutropin (LSH), thyrotropin (TSH) and chorionic gonadotropin (CG), as well as at least two forms of fish gonadotropins. All these hormones consist of two glycosylated chains (alpha and beta). In mammalian gonadotropins, the alpha chain is identical in the four types of hormones, but the beta chains, while homologous, are different. The alpha chains are highly conserved proteins of about 100 amino acid residues which contain ten conserved cysteines all involved in disulfide bonds.

HCG is a dimer glycoprotein that consists of approximately 237 amino acids. HCG is produced during pregnancy and is secreted by the placenta. The HCG-α polypeptide consists of approximately 92 amino acids and is used to detect testicular cancer, pituitary adenomas and malignant endocrine gastroenteropancreatic tumors. The HCG-β polypeptide consists of approximately 115 amino acids and is a tumor marker used to detect testicular tumors. Expression of human chorionic gonadotrophin is associated with trophoblastic, testicular and other malignancies such as bladder, pancreatic, cervical, breast and prostate cancer. In the prostate, however, HCG expression, associated with neuroendocrine cells, is also found in normal tissue. Among six highly homologous genes that all encode the beta-subunit of HCG, the beta 7 gene is reportedly the only gene expressed in several non-transformed tissues (Span, et al., J Endocrinol 172(3):489-95 (2002).

There are five disulphide bonds in the a-subunit and six in the B-subunit. The chemical assignment of the disulphide pairings has been extensively studied (see, Ryan et al., Rec. Prog. Honn. Res. 43:383 (1987)). The amino acid sequences of HCG-α and HCG-β are disclosed in Stockell et al., Biochem. J. 287:665 (1992) and the Protein Data Bank (PDB), each of which is incorporated herein by reference in its entirety.

The three-dimensional structure of human chorionic gonadotropin shows that each of its two different subunits has a similar topology, with three disulphide bonds forming a cysteine knot (Lapthom, et al., Nature 369:455-461 (1994)). The heterodimer is stabilized by a segment of the β-subunit which wraps around the α-subunit and is covalently linked like a seat belt by the disulphide Cys 26-Cys 110. This extraordinary feature appears to be essential not only for the association of these heterodimers but also for receptor binding by the glycoprotein hormones.

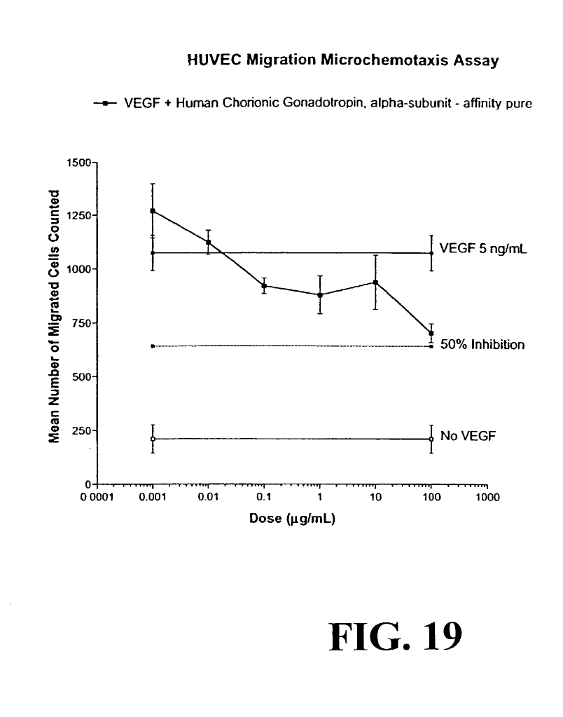

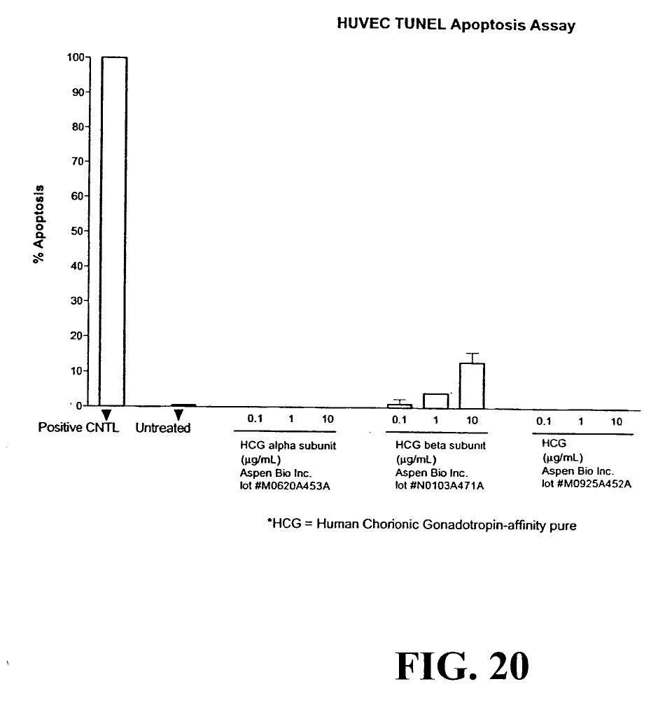

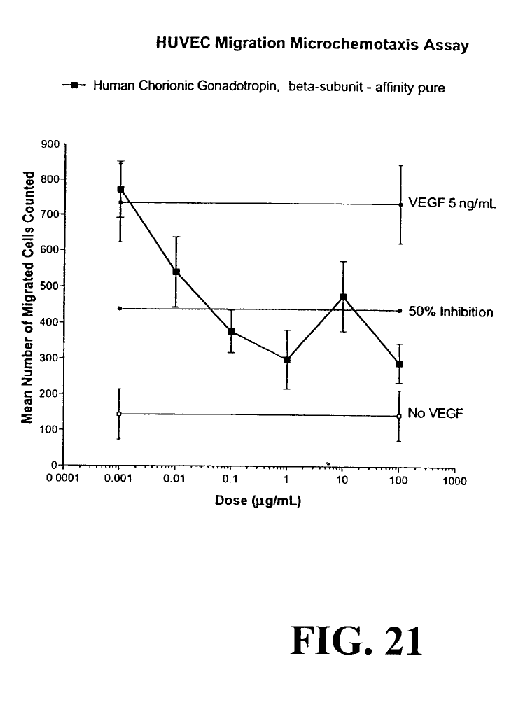

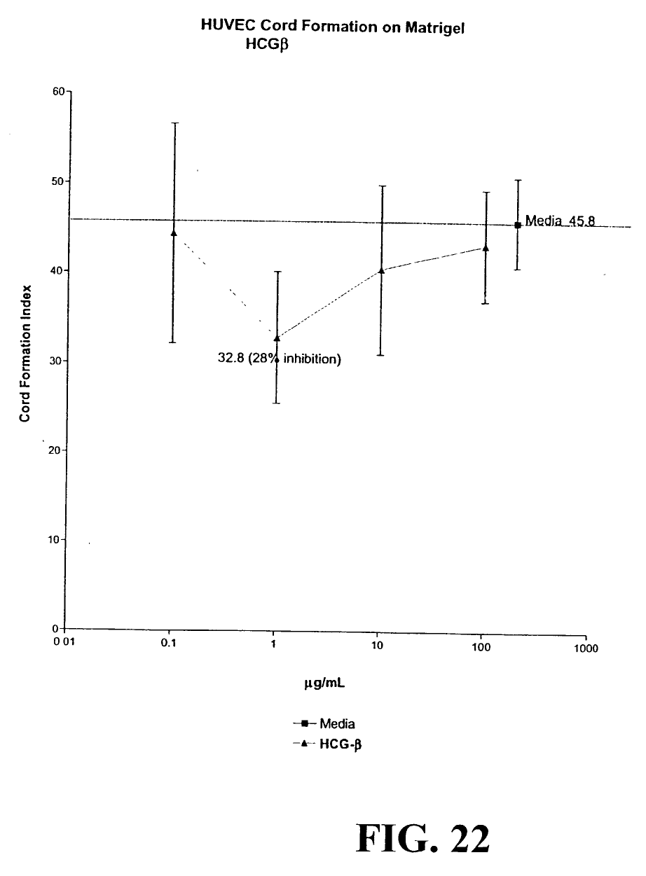

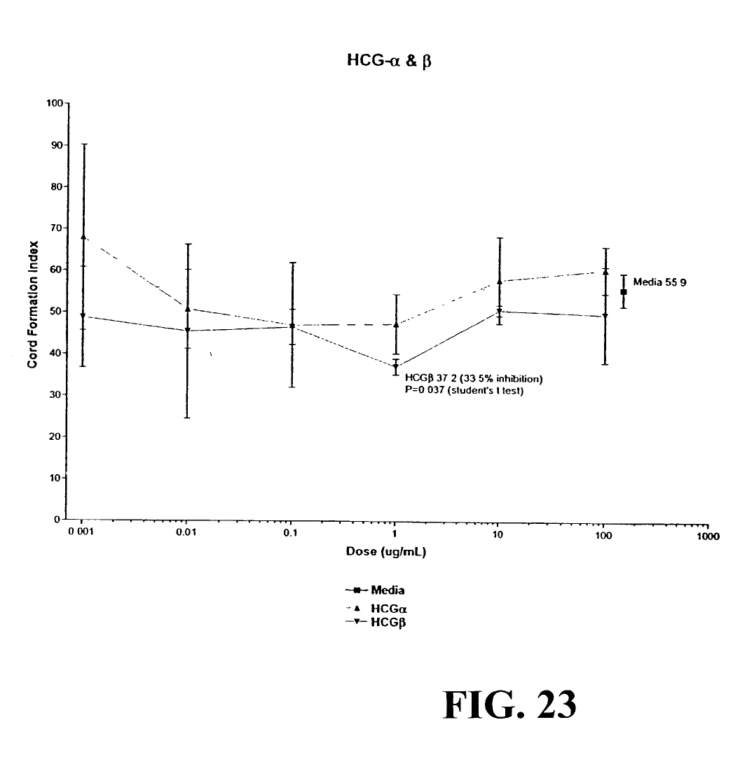

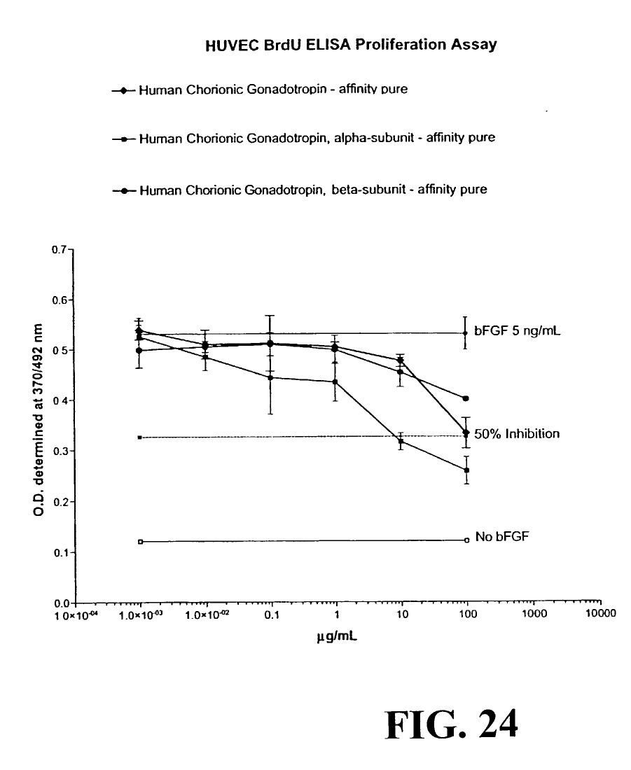

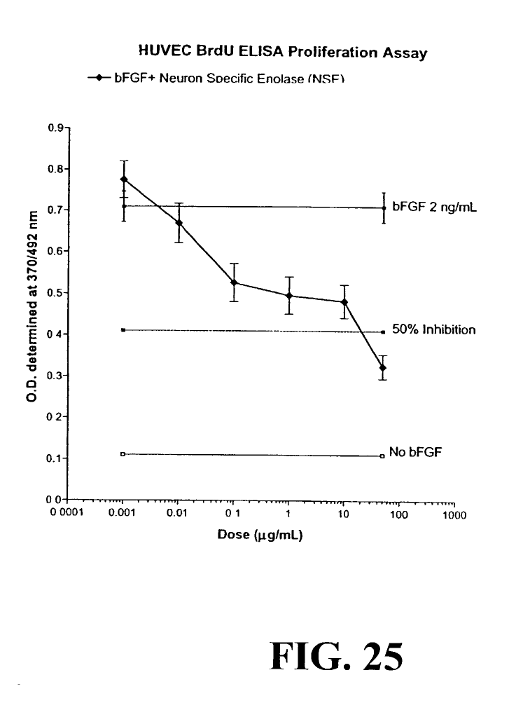

The invention disclosed herein demonstrates for the first time that both HCG-α, and HCG-β exhibit antiangiogenic activity, albeit with a different mechanism of action, as demonstrated in FIGS. 19-24 of the present application.

According to yet another embodiment of the invention, cancer marker neuron specific enolase (NSE) is used as a regulator of angiogenesis. NSE is the neuron-specific isomer of the glycolytic enzyme 2-phospho-D-glyerate hydrolase or enolase. NSE is a marker for small cell lung carcinoma and is widely used serum marker for neuroendocrine tissues.

The enolases (phosphopyruvate hydratase, EC 4.2.1.11) are enzymes that catalyze the interconversion of 2-phosphoglycerate to phosphoenolpyruvate in the glycolytic pathway. The functional enzyme is a homodimer made up of subunits referred to as α, β, and γ (Zomzely-Neurath (1983) Enolase. In ‘Handbook of Neurochemistry38 (A. Lajtha, Ed.), Vol. 4, 2nd ed., pp. 403-433, Plenum Press, New York). These subunits are closely related to one another, exhibiting strong similarity at the amino acid level (more than 80%). Moreover, the polypeptide sequences predicted from enolase-encoding cDNAs isolated from different species show a high degree of evolutionary conservation (Segil et al, Biochem. J. 251: 31-39 (1988); and McAleese et al., Eur. J. Biochem. 178: 413-417 (1988). In manmals there are at least three isoforms of enolase characterized by different tissue distributions as well as by distinct biochemical and immunological properties (see, Rider and Taylor, Biochim, Biophys. Acta 365: 285-300 (1974)).

It has been reported that at least three genes encode the different isoforms of the glycolytic enzyme enolase (Cali et al., Genomics 10: 157-165 (1991)) content of which is incorporated herein by reference in its entirety. Cali et al., have isolated the gene for the human γ or neuron-specific enolase and determined the nucleotide sequence from upstream to the 5′ end to beyond the polyadenylation site. The gene is reported to contain 12 exons distributed over 9213 nucleotides. Introns occur at positions identical to those reported for the homologous rat gene as well as for the human a or normeuronal enolase gene, supporting the existence of a single ancestor for the members of this gene family. Primer extension analysis indicated that the gene has multiple start sites. The putative promoter region shows a lack of TATA and CAAT boxes, is very G-C-rich, and contains several potential regulatory sequences. A comparison of the 5-flanking region of the human γ enolase gene with the same region of the rat gene revealed a high degree of sequence conservation.

According to yet another embodiment of the invention, Squamous cell carcinoma-associated antigen (SCC) is used as an angiogenic regulator. SCC is relatively tumor-specific, and widely used for monitoring patients with squamous cell carcinoma.

Biological Activity

Cancer markers, including agonists, antagonists, or a biologically active fragment of the cancer markers of the present invention, are used in bioassays to test for one or more biological activities. A biological activity, according to the invention described herein, includes, but is not limited to, regulation of endothelial cell formation and proliferation including, migration, cord formation, apoptosis; antigenicity (ability to bind or compete with a cancer marker of the invention for binding to an antibody or a binding peptide of the cancer marker of the invention); immunogenicity (ability to generate antibody which binds to a cancer marker of the invention); ability to form multimers with a cancer marker of the invention; and/or ability to bind to a receptor or ligand for a cancer marker of the invention.

According to one aspect of the invention, the biological activity of a cancer marker is related to the regulation of endothelial cell proliferation and/or formation. In particular, for determination of biological activity related to endothelial cell proliferation and/or formation, bioassays such as, for example, the human umbilical vein endothelial cell proliferation assay (HUVEC) and the bovine capillary endothelial cell proliferation assay (BCE) are used. Such assays are described in U.S. Pat. No. 5,639,725, which is incorporated herein by reference in its entirety. Other assays used include the chick CAM assay, the mouse corneal assay, and the tumor implant assay. The chick CAM assay is described by O'Reilly et al., Cell 79(2):315 (1994), which is hereby incorporated by reference in its entirety.

According to one embodiment of the invention, the biological activity of the cancer cancers markers is related to the induction of apoptosis. These cancer markers may act either directly, or indirectly to induce apoptosis of proliferative cells and tissues. Without being limited to any particular mechanism of action to induce apoptosis, one possible mechanism of action would be the involvement of the cancer marker in the activation of a death-domain receptor, such as tumor necrosis factor (TNF) receptor-1, CD95 (Fas/APO-1), TNF-receptor-related apoptosis-mediated protein (TRAMP) and TNF-related apoptosis-inducing ligand (TRAIL) receptor-1 and -2 (see, Schulze-Osthoff K, et al., Eur J Biochem 254:439 (1998)). In another embodiment of the present invention, cancer markers induce apoptosis through other mechanisms, such as in the activation of other proteins which will activate apoptosis, or through stimulating the expression of the cancer markers, either alone or in combination with small molecule drugs or adjuvants, such as apoptonin, galectins, thioredoxins, antiinflammatory proteins.

According to a preferred embodiment, cancer markers of the present invention are useful in inhibiting the metastasis of proliferative cells or tissues. Inhibition may occur as a direct result of administering the cancer marker, or indirect result of administration of cancer marker. Such indirect inhibition may include, for example, the activation of the expression of proteins known to inhibit metastasis, for example alpha 4 integrins, (See, i.e., Curr. Top. Microbiol Immunol. 231:125 (1998)). Such therapeutic affects of the present invention may be achieved either alone, or in combination with other antiangiogenic drugs, small molecule drugs, adjuvants, or a combination thereof.

According to another embodiment, the biological activity of the cancer marker of the present invention includes repressing expression of oncogenic genes or antigens. Repressing expression of the oncogenic genes includes, but is not limited to, the suppression of the transcription of the gene, the degradation of the gene transcript (pre-message RNA), the inhibition of splicing, the destruction of the messenger RNA, the prevention of the post-translational modifications of the protein, the destruction of the protein, the inhibition of the normal function of the protein, or a combination thereof.

The biological activity of the cancer marker can also be detected by immunoassays to test for the ability of a molecule to bind or compete with the cancer marker of the invention. Various immunoassays known in the art can be used, including but not limited to, competitive and non-competitive assay systems using techniques such as radioimmunoassay, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (i.e., gel agglutination assays, hemagglutination assays), complement fixation assays, immunofluorescence assays, protein A assays, and immunoelectrophoresis assays.

In addition, assays described herein and otherwise known in the art may routinely be applied to measure the ability of cancer markers of the invention to elicit a biological activity either in vitro or in vivo. Other methods will be known to the skilled artisan and are within the scope of the invention. Thus, the skilled artisan would readily be able to take advantage of the cancer markers and angiogenic regulatory potential of cancer markers, as described herein, for various screening, detection, diagnostic and prognostic assays as well as for pharmacogenomics and methods of treatment.

Therapeutic and Diagnostic Activities

The cancer markers of the invention, including agonists, antagonists, or a biologically active fragment thereof, are useful in treating, preventing, and/or diagnosing of several diseases, disorders, and/or conditions.

According to one aspect, the invention provides for a cancer marker that is useful in regulating proliferation of cells or tissues, either alone, or in combination with an antiangiogenic compound, a cytotoxic agent, or both. In one embodiment, the present invention provides for treatment of diseases, disorders, and/or conditions associated with neovascularization by administration to a subject in need thereof, a pharmaceutical composition containing a cancer marker. Malignant and metastatic conditions which can be treated with the pharmaceutical composition of the invention include, but are not limited to, solid tumors, and cancers described herein and otherwise known in the art (for a review of such disorders, see Fishman et al., Medicine, 2d Ed., J. B. Lippincott Co., Philadelphia (1985)).

Cancers which may be treated, prevented, and/or diagnosed with the pharmaceutical composition of the invention include, but are not limited to, solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases, melanomas; glioblastoma, Kaposi's sarcoma; leiomyosarcoma, non-small cell lung cancer, colorectal cancer, advanced malignancies; and blood born tumors such as leukemias.

According to one aspect of the present invention, the pharmaceutical composition is used in treating, preventing, and/or diagnosing diseases, disorders, and/or conditions of angiogenesis not related to cancer. These diseases, disorders, and/or conditions include, but are not limited to benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma, retrolental fibroplasia, rubeosis, retinoblastoma, uvietis and pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

The present invention also provides methods for treating, preventing, and/or diagnosing neovascular diseases of the eye, including for example, neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, Waltman et al, Am. J. Ophthal. 85:704 (1978).

Thus, within one aspect of the present invention methods are provided for treating or preventing neovascular diseases of the eye such as corneal neovascularization (including corneal graft neovascularization), comprising the step of administering to a patient in need thereof a therapeutically effective amount of a compound (as described above) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of diseases, disorders, and/or conditions can result in corneal neovascularization, including for example, corneal infections (i.e., trachoma, herpes simplex keratitis, leishmaniasis and onchocerciasis), immunological processes (i.e., graft rejection and Stevens-Johnson's syndrome), alkali burns, trauma, inflammation (of any cause), toxic and nutritional deficiency states, and as a complication of wearing contact lenses.

Within another aspect of the present invention, methods are provided for treating, preventing, and/or diagnosing hypertrophic scars and keloids, comprising the step of administering the pharmaceutical composition of the invention to a hypertrophic scar or keloid.

Within one embodiment of the present invention, pharmaceutical composition is directly injected into a hypertrophic scar or keloid, in order to prevent the progression of these lesions. This therapy is of particular value in the prophylactic treatment of conditions which are known to result in the development of hypertrophic scars and keloids (i.e., burns), and is preferably initiated after the proliferative phase has progressed, but before hypertrophic scar or keloid development.

Within one aspect, the present invention provides for the use of the cancer markers as a birth control agent by reducing or preventing uterine vascularization required for embryo implantation. Thus, the present invention provides an effective birth control method when an amount of the cancer marker sufficient to prevent embryo implantation is administered to a female. For example, an amount of the cancer marker sufficient to block embryo implantation is administered before or after intercourse, thus providing an effective method of birth control, possibly a “morning after” method. Cancer markers may also be used in controlling menstruation or administered as either a peritoneal lavage fluid or for peritoneal implantation in the treatment of endometriosis.

While not wanting to be bound by this theory, it is believed that inhibition of vascularization of the uterine endometrium interferes with implantation of the blastocyst. Similar inhibition of vascularization of the mucosa of the uterine tube interferes with implantation of the blastocyst, preventing occurrence of a tubal pregnancy. Administration methods may include, but are not limited to, pills, injections (intravenous, subcutaneous, intramuscular), suppositories, vaginal sponges, vaginal tampons, and intrauterine devices. It is also believed that cancer marker administration will interfere with normal enhanced vascularization of the placenta, and also with the development of vessels within a successfully implanted blastocyst and developing embryo and fetus.

According to yet another aspect of the invention, the pharmaceutical composition is utilized in a wide variety of surgical procedures. For example, within one embodiment of the present invention a compositions (in the form of, for example, a spray or film) may be utilized to coat or spray an area prior to removal of a tumor, in order to isolate normal surrounding tissues from malignant tissue, and/or to prevent the spread of disease to surrounding tissues. Within other embodiments of the present invention, compositions (i.e., in the form of a spray) may be delivered via endoscopic procedures in order to coat tumors, or inhibit angiogenesis in a desired locale. Within yet other embodiments of the present invention, surgical meshes which have been coated with antiangiogenic compositions of the present invention may be utilized in any procedure wherein a surgical mesh might be utilized. For example, within one embodiment of the invention a surgical mesh laden with an antiangiogenic composition may be utilized during abdominal cancer resection surgery (i.e., subsequent to colon resection) in order to provide support to the structure, and to release an amount of the antiangiogenic factor.

Within further embodiments of the present invention, methods are provided for treating tumor excision sites, comprising administering the pharmaceutical composition to the resection margins of a tumor subsequent to excision, such that the local recurrence of cancer and the formation of new blood vessels at the site is inhibited. Within one embodiment of the invention, the pharmaceutical composition is administered directly to the tumor excision site (i.e., applied by swabbing, brushing or otherwise coating the resection margins of the tumor with the antiangiogenic compound). Alternatively, the pharmaceutical composition may be incorporated into known surgical pastes prior to administration. Within particularly preferred embodiments of the invention, the pharmaceutical composition is applied after hepatic resections for malignancy, and after neurosurgical operations.

In a specific embodiment, the pharmaceutical composition of the invention affects apoptosis, and therefore, is useful in treating a number of diseases associated with increased cell survival or the inhibition of apoptosis. For example, diseases associated with increased cell survival or the inhibition of apoptosis that could be treated or detected by the pharmaceutical composition of the invention, include cancers such as, follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer), autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection.

Additional diseases or conditions associated with increased cell survival that could be treated or detected by pharmaceutical composition of the invention, include, but are not limited to, progression, and/or metastases of malignancies and related disorders such as leukemia (including acute leukemias (i.e., acute lymphocytic leukemia, acute myelocytic leukemia (including myeloblastic, promyelocytic, myelomonocytic, monocytic, and erythroleukemia)) and chronic leukemias (i.e., chronic myelocytic (granulocytic) leukemia and chronic lymphocytic leukemia)), polycythemia vera, lymphomas (i.e., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated or detected by the pharmaceutical composition of the invention, include AIDS; neurodegenerative disorders (such as Alzheimer's disease, Parkinson's disease, amyotrophic lateral sclerosis, retinitis pigmentosa, Cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (i.e., hepatitis related liver injury, ischemia/reperfusion injury, cholestosis (bile duct injury) and liver cancer); toxin-induced liver disease (such as that caused by alcohol), septic shock, cachexia and anorexia.

In preferred embodiments, the pharmaceutical composition of the invention is used to inhibit growth, progression, and/or metastisis of cancers, in particular those listed above.

Cancer Markers and Cytotoxic Agents

In one embodiment, the invention provides a method for the specific destruction of cells (i.e., the destruction of tumor cells) by administering the cancer marker of the invention in association with toxins or cytotoxic prodrugs.

As used herein “toxin” refers to compounds that bind and activate endogenous cytotoxic effector systems, radioisotopes, holotoxins, modified toxins, catalytic subunits of toxins, or any molecules or enzymes not normally present in or on the surface of a cell that define conditions that cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin.

As used herein “cytotoxic prodrug” refers to a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

Drug Screening

Further contemplated within the present invention is the use of drug screening methods to modify the activity of the antiangiogenic cancer markers. This invention is particularly useful for screening therapeutic compounds by using a cancer marker, or a binding fragment thereof, in any of a variety of drug screening techniques. In one embodiment, the screening method is used for identifying polypeptides, nucleotide and/or small molecules that bind cancer markers of the invention. Such a drug screening method would include, for example, contacting the polypeptide-based cancer marker of the present invention with a selected compound(s) suspected of having antagonist or agonist activity, and assaying the activity of these compounds following binding.

These binding molecules are useful, for example, as agonists and antagonists of cancer markers of the invention. Such agonists and antagonists can be used, in accordance with the invention, in the therapeutic embodiments described in detail, below.

The compounds employed in such a test may be affixed to a solid support, expressed on a cell surface, free in solution, or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. One may measure, for example, the formulation of complexes between the agent being tested and a polypeptide of the present invention.

Thus, the present invention provides methods of screening for drugs or any other agents which affect activities mediated by the cancer markers of the present invention. These methods comprise contacting such an agent with a cancer marker of the present invention and assaying for the presence of a complex between the agent and the cancer marker, by methods well known in the art. In such a competitive binding assay, following incubation, free agent is separated from that present in bound form, and the amount of free label is a measure of the ability of a particular agent to bind to the cancer marker of the present invention.

Another technique for drug screening provides high throughput screening for compounds having suitable binding affinity to the cancer marker of the present invention, and is described in great detail in European Patent Application 84/03564, published on Sep. 13, 1984, which is incorporated herein by reference. Briefly stated, large numbers of different small peptide test compounds are synthesized on a solid substrate, such as plastic pins or some other surfaces. The peptide test compounds are reacted with the cancer marker of the present invention and washed. Bound polypeptides are then detected by methods well known in the art. Purified polypeptides are coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies are used that are capable of binding cancer markers of the present invention and specifically compete with a test compound for binding to cancer markers. In this manner, the antibodies are used to detect the presence of any peptide which shares one or more antigenic epitopes with a cancer marker of the invention.

Alternatively, one may also separate a plurality of cancer markers into substantially separate fractions comprising a subset of or individual cancer markers. For instance, one can separate the plurality of cancer markers by gel electrophoresis, column chromatography, or like method known to those of ordinary skill for the separation of molecules. The individual cancer marker can also be produced by a transformed host cell in such a way as to be expressed on or about its outer surface (i.e., a recombinant phage). Individual isolates can then be “probed” by the caner marker of the invention, optionally in the presence of an inducer should one be required for expression, to determine if any selective affinity interaction takes place between the cancer marker of the invention and the individual clone. In this manner, positive clones could be identified from a collection of transformed host cells of an expression library, which harbor a DNA construct encoding a polypeptide having a selective affinity for a cancer marker of the invention.

Furthermore, the amino acid sequence of the polypeptide having a selective affinity for the cancer marker of the invention can be determined directly by conventional means or the coding sequence of the DNA encoding the polypeptide can frequently be determined more conveniently. The primary sequence can then be deduced from the corresponding DNA sequence. If the amino acid sequence is to be determined from the polypeptide itself, one may use microsequencing techniques. The sequencing technique may include mass spectroscopy.

In certain situations, it may be desirable to wash away any unbound polypeptide of the invention, or alternatively, unbound polypeptides, from a mixture of the polypeptide of the invention and the plurality of polypeptides prior to attempting to determine or to detect the presence of a selective affinity interaction. Such a wash step may be particularly desirable when the polypeptide of the invention or the plurality of polypeptides is bound to a solid support.

The plurality of molecules provided according to this method may be provided by way of diversity libraries, such as random or combinatorial peptide or non-peptide libraries which can be screened for molecules that specifically bind to a polypeptide of the invention. Many libraries are known in the art that can be used, i.e., chemically synthesized libraries, recombinant (i.e., phage display libraries), and in vitro translation-based libraries. Examples of chemically synthesized libraries are described in Fodor et al., Science 251:767-773 (1991); Houghten et al., Nature 354:84-86 (1991); and Brenner and Lemer, Proc. Natl. Acad. Sci. USA 89:5381-5383 (1992), among others.

Examples of phage display libraries are described in Scott and Smith, Science 249:386-390 (1990); Devlin et al., Science 249:404-406 (1990); and Christian et al., J. Mol. Biol. 227:711-718 (1992), among others. In vitro translation-based libraries are described, for example, in PCT Publication No. WO 91/05058 dated April 18, (1991); and Mattheakis et al., Proc. Natl. Acad. Sci. USA 91:9022-9026 (1994), among others.

By way of examples of non-peptide libraries, a benzodiazepine library (see, i.e., Bunin et al., Proc. Natl. Acad. Sci. USA 91:4708-4712 (1994)) can be adapted for use. Peptoid libraries (Simon et al., Proc. Natl. Acad. Sci. USA 89:9367-9371 (1992)) can also be used. Another example of a library that can be used, in which the amide functionalities in peptides have been permethylated to generate a chemically transformed combinatorial library, is described by Ostresh et al., Proc. Natl. Acad. Sci. USA 91:11138-11142 (1994).

The non-peptide libraries that are useful in the present invention are, for example, libraries described by Ecker and Crooke, Bio/Technology 13:351-360 (1995). These libraries use compounds such as, for example, benzodiazepines, hydantoins, piperazinediones, biphenyls, sugar analogs, beta-mercaptoketones, arylacetic acids, acylpiperidines, benzopyrans, cubanes, xanthines, aminimides, and oxazolones among others to form the basis of various libraries.

Non-peptide libraries can be classified broadly into two types: decorated monomers and oligomers. Decorated monomer libraries employ a relatively simple scaffold structure upon which a variety functional groups is added. Often the scaffold will be a molecule with a known useful pharmacological activity. For example, the scaffold might be the benzodiazepine structure.

Non-peptide oligomer libraries utilize a large number of monomers that are assembled together in ways that create new shapes that depend on the order of the monomers. Among the monomer units that have been used are carbamates, pyrrolinones, and morpholinos. Peptoids, peptide-like oligomers in which the side chain is attached to the alpha amino group rather than the alpha carbon, form the basis of another version of non-peptide oligomer libraries. The first non-peptide oligomer libraries utilized a single type of monomer and thus contained a repeating backbone. Recent libraries have utilized more than one monomer, giving the libraries added flexibility.

Screening the libraries can be accomplished by any of a variety of commonly known methods. See, i.e., the following references, which disclose screening of peptide libraries: Parmley and Smith, Adv. Exp. Med. Biol. 251:215-218 (1989); Scott and Smith, id.; Fowlkes et al., BioTechniques 13:422-427 (1992); Oldenburg et al., Proc. Natl. Acad. Sci. USA 89:5393-5397 (1992); and Yu et al., Cell 76:933-945 (1994), among others. In a specific embodiment, screening to identify a molecule that binds a polypeptide of the invention can be carried out by contacting the library members with a polypeptide of the invention immobilized on a solid phase and harvesting those library members that bind to the polypeptide of the invention. Examples of such screening methods, termed “panning” techniques are described by way of example in Fowlkes et al., id. In another embodiment, the two-hybrid system for selecting interacting proteins in yeast (Fields and Song, Nature 340:245-246 (1989)); can be used to identify molecules that specifically bind to a polypeptide of the invention.

Where the polypeptide of the invention binding molecule is a polypeptide, the polypeptide can be conveniently selected from any peptide library, including random peptide libraries, combinatorial peptide libraries, or biased peptide libraries. The term “biased” is used herein to mean that the method of generating the library is manipulated so as to restrict one or more parameters that govern the diversity of the resulting collection of molecules.

Pharmaceutical Composition Formulation and Mode of Administration

The present invention also provides pharmaceutical compositions comprising a therapeutically effective amount of a cancer marker compound, and a pharmaceutically acceptable carrier. The pharmaceutical composition also refers to a composition that additionally contains an antiangiogenic polypeptide and/or cytotoxic agent.

According to one embodiment, the cancer markers of the invention having angiogenic regulatory activity described are provided as isolated and substantially purified compounds in pharmaceutically acceptable formulations using formulation methods known to those of ordinary skill in the art. These formulations can be administered by standard routes.

In a specific embodiment, the term “pharmaceutically acceptable” means approved by a regulatory agency of the Federal or a state government or listed in the U.S. Pharmacopcia or other generally recognized pharmacopcia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In general, the combinations may be administered by the transdermal, intraperitoneal, intracranial, intracerebroventricular, intracerebral, intravaginal, intrauterine, oral, rectal, ophthalmic (including intravitreal or intracameral), nasal, topical (including buccal and sublingual), parenteral (including subcutaneous, intraperitoneal, intramuscular, intravenous, intradermal, intracranial, intratracheal, and epidural) administration.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In addition, the cancer markers may be incorporated into biodegradable polymers allowing for sustained release of the compound, the polymers being implanted in the vicinity of where drug delivery is desired, for example, at the site of a tumor or implanted so that the composition is slowly released systemically. Osmotic mini-pumps may also be used to provide controlled delivery of high concentrations of the composition of cancer markers through cannulae to the site of interest, such as directly into a metastatic growth or into the vascular supply to that tumor. The biodegradable polymers and their use are described, for example, in detail in Brem et al., J. Neurosurg. 74:441-446 (1991), which is hereby incorporated by reference in its entirety.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The pharmaceutical composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Within particularly preferred embodiments of the invention, the pharmaceutical composition may be prepared for topical administration in saline (combined with any of the preservatives and antimicrobial agents commonly used in ocular preparations), and administered in eyedrop form. The solution or suspension may be prepared in its pure form and administered several times daily. Alternatively, the pharmaceutical composition, prepared as described above, may also be administered directly to the cornea. Within preferred embodiments, the antiangiogenic composition is prepared with a muco-adhesive polymer which binds to cornea. Within further embodiments, the antiangiogenic factors or antiangiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the pharmaceutical composition may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted comeas. In a sustained-release form injections might only be required 2-3 times per year. A steroid could also be added to the injection solution to reduce inflammation resulting from the injection itself.

Within another embodiment of the present invention, methods are provided for treating or preventing neovascular glaucoma, comprising the step of administering to a patient a therapeutically effective amount of the pharmaceutical composition of the invention to the eye, such that the formation of blood vessels is inhibited. In one embodiment, the compound may be administered topically to the eye in order to treat or prevent early forms of neovascular glaucoma.

Within other embodiments, the compound may be implanted by injection into the region of the anterior chamber angle. Within other embodiments, the compound may also be placed in any location such that the compound is continuously released into the aqueous humor.

Within one aspect of the present invention, methods are provided for treating or preventing proliferative diabetic retinopathy, comprising the step of administering to a patient a therapeutically effective amount of a polynucleotide, polypeptide, antagonist and/or agonist of the cancer marker to the eyes, such that the formation of blood vessels is inhibited.

Within particularly preferred embodiments of the invention, proliferative diabetic retinopathy may be treated by injection into the aqueous humor or the vitreous, in order to increase the local concentration of the polynucleotide, polypeptide, antagonist and/or agonist in the retina. Preferably, this treatment should be initiated prior to the acquisition of severe disease requiring photocoagulation.

Within another aspect of the present invention, methods are provided for treating or preventing retrolental fibroplasia, comprising the step of administering to a patient a therapeutically effective amount of the pharmaceutical composition of the invention to the eye, such that the formation of blood vessels is inhibited. The compound may be administered topically, via intravitreous injection and/or via intraocular implants.

Within one aspect of the present invention, the pharmaceutical composition of the invention may be administered to the resection margin of a wide variety of tumors, including for example, breast, colon, brain and hepatic tumors. For example, within one embodiment of the invention, antiangiogenic compounds may be administered to the site of a neurological tumor subsequent to excision, such that the formation of new blood vessels at the site are inhibited.

Various delivery systems are known and can be used to administer a compound of the invention, i.e., encapsulation in liposomes, microparticles, microcapsules, recombinant cells capable of expressing the compound, receptor-mediated endocytosis (see, i.e., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (i.e., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, i.e., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, i.e., in conjunction with a wound dressing after surgery, by injection, by means of a catheter, by means of a suppository, or by means of an implant, the implant being of a porous, non-porous, or gelatinous material, including membranes, such as sialastic membranes, or fibers. Preferably, when administering a protein, including an antibody, of the invention, care must be taken to use materials to which the protein does not absorb or otherwise interact.

In one embodiment, the compound or composition can be delivered in a controlled release system. In one embodiment, a pump may be used (see, Sefton, Biomed. Eng. 14:201 (1987)). In another embodiment, polymeric materials can be used (see, Ranger and Peppas, J., Macromol. Sci. Rev. Macromol. Chem. 23:61 (1983); and Levy et al., Science 228:190 (1985)). In yet another embodiment, a controlled release system can be placed in proximity of the therapeutic target, i.e., the brain, thus requiring only a fraction of the systemic dose. Other controlled release systems are discussed in the review by Langer, Science 249:1527-1533 (1990).

The amount of the compound of the invention which will be effective in the treatment, inhibition and prevention of a disease or disorder associated with expression and/or activity of a polypeptide of the invention can be determined by standard clinical techniques. In addition, in vitro assays may optionally be employed to help identify optimal dosage ranges.

In particular, the dosage of the cancer marker composition of the present invention will depend on the disease state or condition being treated and other clinical factors such as weight and condition of the human or animal and the route of administration of the compound. The precise dose to be employed in the formulation, therefore, should be decided according to the judgment of the practitioner and each patient's circumstances. Effective doses may be extrapolated from dose-response curves derived from in vitro or animal model test systems.

For treating humans or animals, between approximately 0.5 to 500 mg/kilogram is typical broad range for administering the pharmaceutical composition of the invention. The methods of the present invention contemplate single as well as multiple administrations, given either simultaneously or over an extended period of time. It is to be understood that the present invention has application for both human and veterinary use.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Gene Therapy/Nucleotide-Based Cancer Markers

In a specific embodiment, where the compound of the invention is a nucleic acid encoding a protein, the nucleic acid can be administered in vivo to promote expression of its encoded protein. One preferred embodiment utilizes compounds of the present invention to inhibit aberrant cellular division, by gene therapy using the polypeptide and/or nucleotide-based cancer markers of the invention. Thus, the present invention provides a method for treating or preventing cell proliferative diseases, disorders, and/or conditions by inserting into an abnormally proliferating cell a polynucleotide of the present invention, wherein said polynucleotide represses said expression.

Another embodiment of the present invention provides a method of treating or preventing cell proliferative diseases, disorders, and/or conditions in individuals comprising administration of one or more active gene copies encoding the cancer marker of the present invention to an abnormally proliferating cell or cells. In a preferred embodiment, polynucleotides of the present invention is a DNA construct comprising a recombinant expression vector effective in expressing a DNA sequence encoding the cancer markers. In another preferred embodiment of the present invention, the DNA construct encoding the cancer marker of the present invention is inserted into cells to be treated utilizing a retrovirus, or more preferably an adenoviral vector (see, Nabel et al., PNAS, 96:324-326 (1999)), which is hereby incorporated by reference. In a most preferred embodiment, the viral vector is defective and will only transform proliferating cells.

In another preferred embodiment, the polynucleotides of the present invention inserted into proliferating cells either alone, or in combination with or fused to other polynucleotides, can then be modulated via an external stimulus (i.e. magnetic, specific small molecule, chemical, or drug administration, etc.), which acts upon the promoter upstream of said polynucleotides to induce expression of the encoded protein product. As such the beneficial therapeutic affect of the present invention may be expressly modulated.

In a specific embodiment where the compound of the invention is a nucleic acid encoding a protein, the nucleic acid can be administered in vivo to promote expression of its encoded protein.

Accordingly, the present invention also encompasses gene therapy whereby a gene encoding a peptide of the invention is regulated in a patient. Various methods of transferring or delivering DNA to cells for expression of the gene product, otherwise referred to as gene therapy, are disclosed in Gene Transfer into Mammalian Somatic Cells in vivo, Yang, Crit. Rev. Biotech. 12(4):335-356 (1992), which is hereby incorporated by reference. Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Strategies for treating these medical problems with gene therapy include therapeutic strategies such as identifying the defective gene and then adding a functional gene to either replace the function of the defective gene or to augment a slightly functional gene. In one embodiment, prophylactic strategies employed that prevents the onset of an angiogenic-related disease or disorder. As an example of a prophylactic strategy, a gene such as that for PSA or CEA may be placed in a patient and thus prevent occurrence of angiogenesis; or a gene that makes tumor cells more susceptible to radiation could be inserted and then radiation of the tumor would cause increased killing of the tumor cells.

Many protocols for transfer of a gene encoding a cancer marker are envisioned in this invention. Transfection of promoter sequences, other than one normally found specifically associated with the cancer marker, or other sequences which would increase production of the cancer marker are also envisioned as methods of gene therapy. An example of this technology is found in Transkaryotic Therapies, Inc., of Cambridge, Mass., using homologous recombination to insert a “genetic switch” that turns on an erythropoietin gene in cells. See, Genetic Engineering News, Apr. 15, 1994. Such “genetic switches” could be used for example to activate kallikreins (or kallikreins receptors) in cells not normally expressing kallikrein (or the kallikrein receptor).

Genetic transformation methods fall into three broad categories: physical (i.e., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses.

In vivo gene transfer involves introducing the DNA into the cells of the patient when the cells are within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of kallikrein DNA or kallikrein regulatory sequences.

Chemical methods of gene therapy involve, for example, a lipid i10 based compound, not necessarily a liposome, to ferry the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct tissue-specific expression of foreign genes, cis-acting regulatory elements or promoters that are known to be tissue specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors that have been used for gene therapy protocols include but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed at by the 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most cell types, precise single copy vector integration into target cell chromosomal DNA, and ease of manipulation of the retroviral genome.

For local administration to abnormally proliferating cells, nucleotide-based cancer marker of the invention may be administered by any method known to those of skill in the art including, but not limited to transfection, electroporation, microinjection of cells, or in vehicles such as liposomes, lipofectin, or as naked polynucleotides, or any other method described throughout the specification. The polynucleotide of the present invention may be delivered by known gene delivery systems such as, but not limited to, retroviral vectors (Hocke, Nature 320:275 (1986)); vaccinia virus system (Chakrabarty et al., Mol. Cell Biol. 5:3403 (1985)), or other efficient DNA delivery systems (Yates et al., Nature 313:812 (1985)) known to those skilled in the art. These references are exemplary only and are hereby incorporated by reference. In order to specifically deliver or transfect cells which are abnormally proliferating and spare non-dividing cells, it is preferable to utilize a retrovirus, or adenoviral, as described in the art and elsewhere herein.

The polynucleotides of the present invention may be delivered directly to cell proliferative disorder/disease sites in internal organs, body cavities and the like by use of imaging devices used to guide an injecting needle directly to the disease site. The polynucleotides of the present invention may also be administered to disease sites at the time of surgical intervention.

In another embodiment, the invention provides a method of delivering compounds and compositions of the invention to targeted cells expressing a receptor for a polypeptide-based cancer marker of the invention, or cells expressing a cell bound form of a polypeptide of the invention. In one embodiment, the invention provides a method for the specific delivery of compositions of the invention to cells by administering the composition of the invention into the targeted cell.

Any amount of the polynucleotides of the present invention may be administered as long as it has a biologically inhibiting effect on the proliferation of the treated cells. Moreover, it is possible to administer more than one of the polynucleotide of the present invention simultaneously to the same site. By “biologically inhibiting” is meant partial or total growth inhibition as well as decreases in the rate of proliferation or growth of the cells. The biologically inhibitory dose may be determined by assessing the effects of the polynucleotides of the present invention on target malignant or abnormally proliferating cell growth in tissue culture, tumor growth in animals and cell cultures, or any other method known to one of ordinary skill in the art.

In another embodiment, the invention provides a method of delivering the pharmaceutical composition containing the polypeptide or nucleotide-based cancer markers, optionally combined with toxins, or prodrugs, to enhance the immunogenicity and/or antigenicity of proliferating cells or tissues, either directly, such as would occur if the composition of the present invention ‘vaccinated’ the immune response to respond to proliferative antigens and immunogens, or indirectly, such as in activating the expression of proteins known to enhance the immune response (i.e. chemokines) to said antigens and immunogens.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of the cells which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of antiangiogenic cancer markers may be accomplished by administering compounds that bind to the cancer marker gene, or control regions associated with the cancer marker gene, or corresponding RNA transcript to modify the rate of transcription or translation. Additionally, cells transfected with a DNA sequence encoding the cancer marker may be administered to a patient to provide an in vivo source of the cancer marker. For example, cells may be transfected with a vector containing a nucleic acid sequence encoding kallikreins. The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. It may be desirable that a recombinant DNA molecule comprising a kallikrein DNA sequence is operatively linked to an expression control sequence to form an expression vector capable of expressing a kallikrein. The transfected cells may be cells derived from the patient's normal tissue, the patient's diseased tissue, or may be non-patient cells.

For example, tumor cells removed from a patient can be transfected with a vector capable of expressing a cancer marker protein, such as, for example, kallikrein protein, of the present invention, and re-introduced into the patient. The transfected tumor cells produce kallikrein levels in the patient that inhibit the growth of the tumor. The gene therapy protocol for transfecting kallikrein into a patient may either be through integration of kallikrein DNA into the genome of the cells, or as a separate replicating or non-replicating DNA construct in the cytoplasm or nucleoplasm of the cell. Kallikrein expression continues for a long-period of time or is reinjected periodically to maintain a desired level of kallikrein protein in the cell, the tissue or organ or a determined blood level.

Polypeptide-Based Cancer Markers

Cancer markers of the invention include polypeptide-based molecules. In general, as used herein, the term polypeptide encompasses variety of modifications, particularly those that are present in polypeptides synthesized by expressing a polynucleotide in a host cell.

It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, or by chemical modification techniques. Modifications which may be present in polypeptides of the present invention include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

It will be appreciated, that polypeptides are not always entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translational natural processes and by entirely synthetic methods, as well.

Modifications occur anywhere in a polypeptide, including the peptide backbone, the amino acid side chains and the amino or carboxyl termini. Blockage of the amino or carboxyl group in a polypeptide, or both, by a covalent modification, occur in a natural or synthetic polypeptides and such modifications are present in polypeptides of the present invention, as well. In general, the nature and extent of the modifications are determined by the host cell's post-translational modification capacity and the modification signals present in the polypeptide amino acid sequence. It will be appreciated that the same type of modification may be present in the same or varying degrees at several sites in a polypeptide. Also, a polypeptide of the invention may contain more than one type of modifications.

Variants of a polypeptide include polypeptides that differ in amino acid sequence from another, reference polypeptide. Generally, differences are limited so that the sequences of the reference and the variant are closely similar overall and, in many regions, identical.

A variant and reference polypeptide may differ in amino acid sequence by one or more substitutions, additions, deletions, fusions and truncations, which may be present in any combination.

The polypeptide-based cancer marker according to the invention includes truncated and/or N-terminally or C-terminally extended forms of the polypeptide, analogs having amino acid substitutions, additions and/or deletions, allelic variants and derivatives of the polypeptide, so long as their sequences are substantially homologous to the native antiangiogenic polypeptide.

Specifically, as will be appreciated by those skilled in the art, the polypeptide-based cancer marker of the invention include those polypeptides having slight variations in amino acid sequences or other properties. Such variations may arise naturally as allelic variations, as disclosed above, due to genetic polymorphism, for example, or may be produced by human intervention (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules. Substitutions may be designed based on, for example, the model of Dayhoff, et. al., Atlas of Protein Sequence and Structure, Nat'l Biomed. Res. Found., Washington, D. C. (1978). These modifications can result in changes in the amino acid sequence, provide silent mutations, modify a restriction site, or provide other specific mutations. The peptide-based cancer marker may comprise one or more selected antigenic determinants of endostatin or angiostatin peptides, possess catalytic activity exhibited by their native protein or alternatively lack such activity, mimic angiostatin or endostatin binding regions, or the like.

The conserved and variable sequence regions of a native antiangiogenic polypeptide and the homology thereof can be determined by techniques known to the skilled artisan, such as sequence alignment techniques. For example, the determination of percent identity between two sequences can also be accomplished using a mathematical algorithm.

A preferred, non-limiting example of a mathematical algorithm utilized for the comparison of two sequences is the algorithm of Karlin and Altschul, Proc. Natl. Acad. Sci. USA, 87:2264-2268 (1990) modified as in Karlin and Altschul, Proc. Natl. Acad. Sci. USA, 90:5873-5877 (1993). Such an algorithm is incorporated into the NBLAST and XBLAST programs of Altschul, et al., J. Mol. Biol. 215:403-410 (1990). BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., Nucleic Acids Res. 25:3389-3402 (1997). Alternatively, PSI-Blast can be used to perform an iterated search which detects distant relationships between molecules (Id.) When utilizing BLAST, Gapped BLAST, and PSI-Blast programs, the default parameters of the respective programs (i.e., XBLAST and NBLAST) can be used (see, http://www.ncbi.nlm.nih.gov).

Another preferred, non-limiting example of a mathematical algorithm utilized for the comparison of sequences is the algorithm of Myers and Miller, CABIOS 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “substantially homologous” as used herein includes, those sequences which have at least about 50% homology, preferably at least 60-70%, more preferably at least about 70-80% homology and most preferably at least about 95% or more homology to the amino acid sequence of a native antiangiogenic peptide and still retain at least some biological activity of the native antiangiogenic peptide. By biological activity is meant the ability to inhibit endothelial cell growth in vitro, to specifically bind antibodies that bind to the native antiangiogenic protein, and/or to elicit antibodies that also bind to the native protein.

Fusion Protein

As one of skill in the art will appreciate, and as discussed above, the cancer marker of the invention can be fused to a heterologous polypeptide sequences. For example, the cancer marker of the present invention (including fragments or variants thereof), may be fused to one or more additional cancer markers, or other antiangiogenic peptides. In a preferred embodiment, endostatin peptides, angiostatin peptides, or a combination thereof is fused with one or more cancer markers. In a preferred embodiment, cancer marker of the present invention are fused with human recombinant angiostatin kringles 1-3, human recombinant endostatin, or a combination thereof.

This invention also relates to genetically engineered soluble fusion proteins comprised of an antiangiogenic polypeptide, for example, endostatin, angiostatin or a portion thereof, a cancer marker polypeptide and of various portions of a membrane bound protein, such as for example protein disulfide isomerase (PDI). This invention further relates to processes for the preparation of these fusion proteins by genetic engineering, and to polynucleotides encoding such fusion proteins.

Nucleic acids encoding the above cancer marker or other antiangiogenic peptides can also be recombined with a gene of interest as an epitope tag (i.e., the hemagglutinin (“HA”) tag or flag tag) to aid in detection and purification of the expressed polypeptide. For example, a system described by Janknecht et al. allows for the ready purification of non-denatured fusion proteins expressed in human cell lines (See, for example, Janknecht et al., Proc. Natl. Acad. Sci. USA 88:8972-897 (1991)). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). DNA shuffling involves the assembly of two or more DNA segments by homologous or site-specific recombination to generate variation in the polynucleotide sequence.

In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

Agonist/Antagonist, Antibodies and Binding Peptides of Cancer Markers

Antagonist/agonist antibodies and/or binding peptides of the cancer marker may be employed to augment the biological activity of the cancer marker of the invention. For example, an agonist or antagonist may increase or decrease inhibition of the growth and proliferation of neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hypervascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat, prevent, and/or diagnose the diseases described herein.

Thus, the invention provides a method of treating or preventing diseases, disorders, and/or conditions, including but not limited to the diseases, disorders, and/or conditions listed throughout this application, associated with over-expression of a polynucleotide of the present invention by administering to a patient (a) an antisense molecule directed to the polynucleotide encoding the cancer marker of the present invention, and/or (b) a ribozyme directed to the polynucleotide encoding the cancer marker of the present invention.

Systematic substitution of amino acids within the synthesized peptides yields high affinity peptide agonists and antagonists to a cancer marker receptor that enhance or diminish cancer marker binding to its receptor. Such agonists are used to suppress the growth of primary and metastatic tumors, thereby limiting the spread of cancer. Antagonists to antiangiogenic cancer markers are applied in situations of inadequate vascularization, to block the inhibitory effects of the cancer marker and possibly promote angiogenesis. This treatment may have therapeutic effects to promote wound healing in diabetics. For example, an antagonist of alpha-1 anti-chymotrypsin (ACT) can be used as an antiangiogenic drug.

Potential antagonists according to the invention also include catalytic RNA, or a ribozyme (see, Sarver et al, Science 247:1222-1225 (1990)). While ribozymes that cleave mRNA at site specific recognition sequences can be used to destroy mRNAs corresponding to the polynucleotides of the invention, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within each nucleotide sequence disclosed in the sequence listing. Preferably, the ribozyme is engineered so that the cleavage recognition site is located near the 5′ end of the mRNA corresponding to the polynucleotides of the invention; i.e., to increase efficiency and minimize the intracellular accumulation of non-functional mRNA transcripts.

Antibodies that specifically bind polypeptide-based cancer markers can be employed to modulate endothelial-dependent processes such as reproduction, development, and wound healing and tissue repair. In addition, antisera directed to the Fab regions of cancer markers antibodies can be administered to block the ability of endogenous cancer marker antisera to bind the cancer marker.

Antibodies specific for the cancer marker are made according to techniques and protocols well known in the art. The antibodies may be either polyclonal or monoclonal. The antibodies are utilized in well know immunoassay formats, such as competitive and non-competitive immunoassays, including ELISA, sandwich immunoassays and radioimmunoassays (RIAs), to determine the presence or absence of the endothelial proliferation inhibitors of the present invention in body fluids. Examples of body fluids include but are not limited to semen, blood, serum, peritoneal fluid, pleural fluid, cerebrospinal fluid, uterine fluid, saliva, and mucus.

Conversely, blockade of the polypeptide-based cancer marker receptors with the polypeptide analogs which act as receptor antagonists, may promote angiogenic activity such as endothelialization and vascularization. Such effects may be desirable in situations of inadequate vascularization of the uterine endometrium and associated infertility, wound repair, healing of cuts and incisions, treatment of vascular problems in diabetics, especially retinal and peripheral vessels, promotion of vascularization in transplanted tissue including muscle and skin, promotion of vascularization of cardiac muscle especially following transplantation of a heart or heart tissue and after bypass surgery, promotion of vascularization of solid and relatively avascular tumors for enhanced cytotoxin delivery, and enhancement of blood flow to the nervous system, including but not limited to the cerebral cortex and spinal cord. A combination of the cancer marker antagonists may be co-applied with stimulators of angiogenesis to increase vascularization of tissue.

Nucleotide-Based Cancer Markers

This invention also encompasses nucleic acid sequences that correspond to, and code for the cancer markers of the invention. The nucleotide-based cancer marker of the present invention are useful in modulating angiogenic processes in vivo, and for diagnosing, preventing and treating endothelial cell-proliferation-related diseases and conditions.

Nucleotide-based cancer markers are prepared based upon the knowledge of the amino acid sequence of the encoded peptide, and the art recognized correspondence between triplet codons and amino acids. Because of the degeneracy of the genetic code, wherein a base in a triplet codon may vary yet still code for the same amino acid, many different possible coding nucleic acid sequences are derivable for any particular protein or peptide fragment.

Nucleic acid sequences are synthesized using automated systems well known in the art. Either the entire sequence may be synthesized or a series of smaller oligonucleotides are made and subsequently ligated together to yield the full length sequence. Alternatively, the nucleic acid sequence may be derived from a gene bank using oligonucleotides probes designed based on the N-terminal amino acid sequence and well known techniques for cloning genetic material.

The nucleotide-based cancer marker of the invention, as described and disclosed herein, generally refers to any polyribonucleotide or polydeoxyribonucleotide, which may be unmodified RNA or DNA or modified RNA or DNA. Thus, for instance, polynucleotide as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide, as used herein, refers to triple-stranded regions comprising RNA or DNA or both RNA and DNA. As used herein, the term polynucleotide also includes DNAs or RNAs as described above that contain one or more modified bases. Thus, DNAs or RNAs with backbones modified for stability or for other reasons are polynucleotides, as that term is intended herein. Moreover, DNAs or RNAs comprising unusual bases, such as inosine, or modified bases, such as tritylated bases of 8 amino adenine bases, to name just a few examples, are polynucleotides, as the term is used herein. It will be appreciated that a great variety of modifications have been made to DNA and RNA that serve many useful purposes known to those of skill in the art. The term polynucleotide, as it is employed herein, embraces such chemically, enzymatically or metabolically modified forms of polynucleotide.

Polynucleotides of the present invention encode, for example, the coding sequence for the mature polypeptide, the coding sequence for the mature polypeptide and additional coding sequences; and the coding sequence of the mature polypeptide, with or without the aforementioned additional coding sequences, together with additional, non-coding sequences. Examples of additional coding sequence include, but are not limited to, sequences encoding a leader or secretory sequence, such as a pre-, pro- or prepro-protein sequence. Examples of additional non-coding sequences include, but are not limited to, introns and non-coding 5′ and 3′ sequences, such as the transcribed, non-translated sequences that play a role in transcription, and mRNA processing, including splicing and polyadenylation signals, for example, for ribosome binding and stability of mRNA.

The polynucleotides also encode a polypeptide which is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may facilitate protein trafficking, may prolong or shorten protein half-life or may facilitate manipulation of a protein for assay or production, among other things. As generally is the case in situ, the additional amino acids may be processed away from the mature protein by cellular enzymes.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

In sum, a polynucleotide of the present invention encodes, for example, a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

“Variant(s)” of polynucleotides or polypeptides, as the term is used herein, are polynucleotides or polypeptides that differ from a reference polynucleotide or polypeptide, respectively. Variants in this sense are described below and elsewhere in the present disclosure in greater detail.

Variants include polynucleotides that differ in nucleotide sequence from another, reference polynucleotide. Generally, differences are limited so that the nucleotide sequences of the reference and the variant are closely similar overall and, in many regions, identical. As noted below, changes in the nucleotide sequence of the variant may be silent. That is, they may not alter the amino acids encoded by the polynucleotide. Where alterations are limited to silent changes of this type, a variant will encode a polypeptide with the same amino acid sequence as the reference. As also noted below, changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence, as discussed below.

Coding sequences which provide additional functionalities are also incorporated into the polynucleotide. Thus, for instance, the expressed polypeptide may be fused to a marker sequence, such as a peptide, which facilitates purification of the fused polypeptide. In certain preferred embodiments of this aspect of the invention, the marker sequence is a hexahistidine peptide, such as that provided in the pQE vector (Qiagen, Inc.). As described in Gentz et al., Proc. Natl. Acad. Sci. 86:821-824 (1989), for instance, hexahistidine provides for convenient purification of the fusion protein. In other embodiments the marker sequence is HA tag. The HA tag corresponds to an epitope derived of influenza hemaglutinin protein, which has been described by Wilson et al., Cell 37:767 (1984), for instance. Many other such tags are commercially available.

In accordance with the foregoing, the term “polynucleotide encoding a polypeptide” as used herein encompasses polynucleotides which include, by virtue of the redundancy of the genetic code, any sequence encoding a polypeptide of the present invention. The term also encompasses polynucleotides that include a single continuous region or discontinuous regions encoding the polypeptide (for example, interrupted by introns) together with additional regions, that also may contain coding and/or non-coding sequences.

The present invention further relates to polynucleotides that hybridize to the herein described sequences. The term “hybridization under stringent conditions” according to the present invention is used as described by Sambrook et al., Molecular Cloning, A Laboratory Manual, Cold Spring Harbor Laboratory Press 1.101-1.104 (1989). Preferably, a stringent hybridization according to the present invention is given when after washing for an hour with 1× SSC and 0.1% SDS at 50° C., preferably at 55° C., more preferably at 62° C., and most preferably at 68° C., and more preferably for 1 hour with 0.2× SSC and 0.1% SDS at 50° C., preferably at 55° C., more preferably at 62° C., and most preferably at 68° C. a positive hybridization signal is still observed. A polynucleotide sequence which hybridizes under such washing conditions with the nucleotide sequence shown in any sequence disclosed herein or with a nucleotide sequence corresponding thereto within the degeneration of the genetic code is a nucleotide sequence according to the invention.

Angiogenic Inhibitory Polypeptides

Angiogenic inhibitory polypeptides of the invention, as described and disclosed herein, encompass polypeptides that have the ability of reducing or inhibiting endothelial cell proliferation.

According to a preferred embodiment of the invention, angiogenic inhibitory polypeptides include, angiostatin (i.e., ANGIOSTATIN®), endostatin (i.e., ENDOSTATIN™), metastatin (i.e., METASTATIN™) HGF, TFPI, anti-invasive factors, retinoic acid and derivatives thereof, paclitaxel, suramin, tissue inhibitor of metalloproteinase-1, tissue inhibitor of metalloproteinase-2, plasminogen activator inhibitor-1, plasminogen activator inhibitor-2, or a combination thereof. Angiostatin and kringle fragment thereof are disclosed in U.S. Pat. No. 5,945,403, content of which is incorporated herein by reference in its entirety.

As used herein, “angiostatin” means a protein derivative of angiostain, or plasminogen, having an endothelial cell proliferation inhibiting activity. The amino acid sequence of an angiostatin can be selected from a portion of murine plasminogen (SEQ ID NO. 1), murine angiostatin (SEQ ID NO. 2), human angiostatin (SEQ ID NO. 3), Rhesus angiostatin (SEQ ID NO. 4), porcine angiostatin (SEQ ID NO. 5), and bovine angiostatin (SEQ ID NO. 6), unless indicated otherwise by the context in which it is used.

As used herein, “kringle 1” means a protein derivative of plasminogen having an endothelial cell inhibiting activity or antiangiogenic activity, and having an amino acid sequence comprising a sequence homologous to kringle 1, exemplified by, but not limited to that of murine kringle 1 (SEQ ID NO. 7), human kringle 1 (SEQ ID NO. 8), Rhesus kringle 1 (SEQ ID NO. 9), porcine kringle 1 (SEQ ID NO. 10), and bovine kringle 1 (SEQ ID NO. 11), murine kringle 1 (SEQ ID NO. 7) corresponds to amino acid positions 103 to 181 of murine plasminogen of SEQ ID NO. 1, and corresponds to amino acid positions 6 to 84 of murine angiostatin of SEQ ID NO. 2. Human kringle 1 (SEQ ID NO. 8), Rhesus kringle 1 (SEQ ID NO. 9), porcine kringle 1 (SEQ ID NO. 10), and bovine kringle 1 (SEQ ID NO. 11) correspond to amino acid positions 6 to 84 of angiostatin of SEQ ID NO. 3, SEQ ID NO. 4, SEQ ID NO. 5, and SEQ ID NO. 6, respectively.

As used herein, “kringle 2” means a protein derivative of plasminogen having an endothelial cell inhibiting activity or antiangiogenic activity, and having an amino acid sequence comprising a sequence homologous to kringle 2, exemplified by, but not limited to that of murine kringle 2 (SEQ ID NO. 12), human kringle 2 (SEQ ID NO. 13), rhesus kringle 2 (SEQ ID NO. 14), porcine kringle 2 (SEQ ID NO. 15), and bovine kringle 2 (SEQ ID NO. 16), unless indicated otherwise by the context in which it is used. Murine kringle 2 (SEQ ID NO. 12) corresponds to amino acid positions 185 to 262 of murine plasminogen of SEQ ID NO. 1, and corresponds to amino acid positions 88 to 165 of murine angiostatin of SEQ ID NO. 2. Human kringle 2 (SEQ ID NO. 13), Rhesus kringle 2 (SEQ ID NO. 14), porcine kringle 2 (SEQ ID NO. 15), and bovine kringle 2 (SEQ ID NO. 16) correspond to amino acid positions 88 to 165 of angiostatin of SEQ ID NO. 3, SEQ ID NO. 4, SEQ ID NO. 5, and SEQ ID NO. 6, respectively.

As used herein, “kringle 3” means a protein derivative of plasminogen having an endothelial cell inhibiting activity or antiangiogenic activity, and having an amino acid sequence comprising a sequence homologous to kringle 3, exemplified by, but not limited to that of murine kringle 3 (SEQ ID NO. 17), human kringle 3 (SEQ ID NO. 18), rhesus kringle 3 (SEQ ID NO. 19), porcine kringle 3 (SEQ ID NO. 20), and bovine kringle 3 (SEQ ID NO. 21). Murine kringle 3 (SEQ ID NO. 17) corresponds to amino acid positions 275 to 352 of murine plasminogen of SEQ ID NO. 1, and corresponds to amino acid positions 178 to 255 of murine angiostatin of SEQ ID NO. 2. Human kringle 3 (SEQ ID NO. 18), rhesus kringle 3 (SEQ ID NO. 19), porcine kringle 3 (SEQ ID NO. 20), and bovine kringle 3 (SEQ ID NO. 21) correspond to amino acid positions 178 to 255 (inclusive) of angiostatin of SEQ ID NO. 3, SEQ ID NO. 4, SEQ ID NO. 5, and SEQ ID NO. 6, respectively.

As used herein, “kringle 4” means a protein derivative of plasminogen having an endothelial cell inhibiting activity or antiangiogenic activity, and having an amino acid sequence comprising a sequence homologous to kringle 4, exemplified by, but not limited to that of murine kringle 4 (SEQ ID NO. 22) and human kringle 4 (SEQ ID NO. 23), unless indicated otherwise by the context in which it is used. Murine kringle 4 (SEQ ID NO. 22) corresponds to amino acid positions 377 to 454 of murine plasminogen of SEQ ID NO. 1.

As used herein “kringle 1-5” means a protein derivative of plasminogen having an endothelial cell inhibiting activity or antiangiogenic activity, and having an amino acid sequence comprising a sequence homologous to kringle 1-5, exemplified by, but not limited to that of murine kringle 1-5 corresponding to amino acid positions 102 to 560 (inclusive) of murine plasminogen of SEQ ID NO. 1. Kringle 5 itself is represented in the murine sequence of plasminogen of SEQ ID NO. 1 at amino acid positions 481-560 (inclusive). The amino acid and corresponding nucleotide sequence of plasminogen is provided in Forsgren et al., FEBS 213:2, 254 (1987), which is hereby incorporated by reference.

As used herein, “kringle 2-3” means a protein derivative of plasminogen having an endothelial cell inhibiting activity or antiangiogenic activity, and having an amino acid sequence comprising a sequence homologous to kringle 2-3, exemplified by, but not limited to that of murine kringle 2-3 (SEQ ID NO. 24), human kringle 2-3 (SEQ ID NO. 25), rhesus kringle 2-3 (SEQ ID NO. 26), porcine kringle 2-3 (SEQ ID NO. 27), and bovine kringle 2-3 (SEQ ID NO. 28), unless indicated otherwise by the context in which it is used. Murine kringle 2-3 (SEQ ID NO. 24) corresponds to amino acid positions 185 to 352 (inclusive) of murine plasminogen of SEQ ID NO. 1, and corresponds to amino acid positions 88 to 255 (inclusive) of murine angiostatin of SEQ ID NO. 2. Human kringle 2-3 (SEQ ID NO. 25), rhesus kringle 2-3 (SEQ ID NO. 26), porcine kringle 2-3 (SEQ ID NO. 27), and bovine kringle 2-3 (SEQ ID NO. 28) correspond to amino acid positions 88 to 255 of angiostatin of SEQ ID NO. 3, SEQ ID NO. 4, SEQ ID NO. 5, and SEQ ID NO. 6, respectively.

As used herein, “kringle 1-3” means a protein derivative of plasminogen having an endothelial cell inhibiting activity or antiangiogenic activity, and having an amino acid sequence comprising a sequence homologous to kringle 1-3, exemplified by, but not limited to that of murine kringle 1-3 (SEQ ID NO. 29), human kringle 1-3 (SEQ ID NO. 30), rhesus kringle 1-3 (SEQ ID NO. 31), porcine kringle 1-3 (SEQ ID NO. 32), and bovine kringle 1-3 (SEQ ID NO. 33), unless indicated otherwise by the context in which it is used. Murine kringle 1-3 (SEQ ID NO. 29) corresponds to amino acid positions 103 to 352 of murine plasminogen of SEQ ID NO. 1, and corresponds to amino acid positions 6 to 255 of murine angiostatin of SEQ ID NO. 2. Human kringle 1-3 (SEQ ID NO. 30), rhesus kringle 1-3 (SEQ ID NO. 31), porcine kringle 1-3 (SEQ ID NO. 32), and bovine kringle 1-3 (SEQ ID NO. 33) correspond to amino acid positions 6 to 255 (inclusive) of angiostatin of SEQ ID NO. 3, SEQ ID NO. 4, SEQ ID NO. 5, and SEQ ID NO. 6, respectively.

As used herein, “kringle 1-2” means a protein derivative of plasminogen having an endothelial cell inhibiting activity or antiangiogenic activity, and having an amino acid sequence comprising a sequence homologous to kringle 1-2, exemplified by, but not limited to that of murine kringle 1-2 (SEQ ID NO. 34), human kringle 1-2 (SEQ ID NO. 35), rhesus kringle 1-2 (SEQ ID NO. 36), porcine kringle 1-2 (SEQ ID NO. 37), and bovine kringle 1-2 (SEQ ID NO. 38), unless indicated otherwise by the context in which it is used. Murine kringle 1-2 (SEQ ID NO. 34) corresponds to amino acid positions 103 to 262 of murine plasminogen of SEQ ID NO. 1, and corresponds to amino acid positions 6 to 165 of murine angiostatin of SEQ ID NO. 2. Human kringle 1-2 (SEQ ID NO. 35), rhesus kringle 1-2 (SEQ ID NO. 36), porcine kringle 1-2 (SEQ ID NO. 37), and bovine kringle 1-2 (SEQ ID NO. 38) correspond to amino acid positions 6 to 165 (inclusive) of angiostatin of SEQ ID NO. 3, SEQ ID NO. 4, SEQ ID NO. 5, and SEQ ID NO. 6, respectively.

As used herein, “kringle 1-4” means a protein derivative of plasminogen having an endothelial cell inhibiting activity or antiangiogenic activity, and having an amino acid sequence comprising a sequence homologous to kringle 1-4, exemplified by, but not limited to that of murine kringle 1-4 (SEQ ID NO. 39) and human kringle 1-4 (SEQ ID NO. 40), unless indicated otherwise by the context in which it is used. Murine kringle 1-4 (SEQ ID NO. 39) corresponds to amino acid positions 103 to 454 of murine plasminogen of SEQ ID NO. 1. Peptide variants of Kringle 1-4 are represented by K 1-4 BKLS sequences represented by SEQ ID NOS 41-42.

Endostatin is approximately 20 kDa, derived from sources including, for example, carboxyl-terminal end of collagen XVIII, which corresponds to the C-terminal 183 amino acid residues of the non-collagenous carboxyl-terminal domain, O'Reilly et al., Cell 88:277(1997); Sim et al., Angiogenesis 3:41(1999). Treatment of human cancer patients with rhEndostatin in phase II clinical trials revealed significant decreases in tumor blood flow and a trend towards reducing tumor metabolism (EntreMed Inc., Rockville, Md., USA, May 14, 2000).

The term endostatin also includes an N terminal fragment of endostatin consisting of the sequence of the first 20N terminal amino acids which is shown in SEQ ID NO. 43. This sequence of the first 20N terminal amino acids corresponds to a C-terminal fragment of newly identified collagen type XVIII.

The Endostatin molecule of the present invention can be recombinantly expressed in any system used to express proteins. Non-limiting examples of such expressions systems include bacterial expression systems, yeast expression systems and insect viral expression systems, as disclosed in U.S. Pat. No. 5,854,205, content of which is incorporated herein in its entirety. A preferred expression system, according to the invention, is a yeast expression system.

It is to be understood that the term endostatin, as defined above and as encompassed within the present invention, includes a variety of forms of endostatin protein, including but not limited to forms that are lengthened or shortened by one or more amino acids, at either or both ends, or at an internal location, of the endostatin protein provided the resulting molecule retains endothelial proliferation inhibiting activity.

The following is an example of, but it is not limited to, functional human endostatin protein, and variants thereof. Human endostatin protein (183 aa) comprising (SEQ ID NO. 52). Human endostatin polynucleotide sequence encoding this protein comprises (SEQ ID NO. 53).

C-terminal variants of Human endostatin protein, include but is not limited to: human endostatin protein: C terminus minus 1 amino acid, lysine, at position 183 (182 aa) comprising (SEQ ID NO. 54); human endostatin protein: C terminus minus 2 amino acids, lysine and serine at positions 183 and 182, respectively (181 aa) comprising (SEQ ID NO. 55). Human endostatin protein: C terminus minus 3 amino acids, lysine, serine, and alanine at positions 183, 182, and 181, respectively (180 aa) comprising SEQ ID NO. 56. The variant represented as C terminus minus 1 amino acid (SEQ ID NO. 54, 182 aa) is the preferred endostatin protein of the present invention.

N-terminal variants of human endostatin protein, include but is not limited to: human endostatin protein: N terminus minus the first 4 N-terminal amino acids (N 1-4, 179 aa) comprising SEQ ID NO. 57. Human endostatin protein: C terminus minus 1 amino acid, lysine, at position 183, and minus the first four amino acids at the N-terminus (N 1-4, 178 aa) comprising SEQ ID NO. 60.

Human endostatin protein was also expressed from clones without the nucleotides encoding for the first 4 (N-terminal) amino acids (N-4) (hESv3) in P. pastoris by amplifying the gene fragment encoding human endostatin protein (SEQ ID NO. 57, N-4) using the forward and reverse primers #359 5′ TCT CTC GAG AAA AGA GAC TTC CAG CCG GTG CTC (SEQ ID NO. 58) and #295 5′ ATC GTC TAG AGC ATC CAG GCG GTG GCT ACT (SEQ ID NO. 63) respectively, using the same strategy as for the gene encoding full length human Endostatin™ protein. The shuttle plasmid used for transforming GS115 was pPIC9K/hESv3/27. The phenotype of the P. pastoris clone expressing rh endostatin protein (N-4) that was selected for study was identified as His+ Mut+.

Method of Making Cancer Markers In vitro

It is contemplated as part of the present invention that cancer markers are isolated from a body fluid such as semen, blood or urine of patients. Alternatively, polypeptide-based cancer markers, such as PSA, HCG, CEA, and NSE among others, can be produced by recombinant DNA methods or synthetic peptide chemical methods that are well known to those of ordinary skill in the art. One example of a method of producing polypeptide or nucleotide-based cancer marker of the invention using recombinant DNA techniques entails the steps of (1) identifying and purifying the polypeptide as discussed above, and as more fully described below, (2) determining the N-terminal amino acid sequence of the purified polypeptide, (3) synthetically generating a DNA oligonucleotide probe that corresponds to the N-terminal amino acid sequence, (4) generating a DNA gene bank from human or other mammalian DNA, (5) probing the gene bank with the DNA oligonucleotide probe, (6) selecting clones that hybridize to the oligonucleotide, (7) isolating the inhibitor gene from the clone, (8) inserting the gene into an appropriate vector such as an expression vector, (9) inserting the gene-containing vector into a microorganism or other expression system capable of expressing the inhibitor gene, and (10) isolating the recombinantly produced inhibitor. The above techniques are more fully described in laboratory manuals such as “Molecular Cloning: A Laboratory Manual” Latest Edition by Sambrook et al., Cold Spring Harbor Press (1989).

Yet another method of producing cancer markers, or biologically active fragments thereof, is by peptide synthesis. For example, once a biologically active fragment of a cancer marker is found, it can be sequenced, for example by automated peptide sequencing methods. Alternatively, once the gene or DNA sequence which codes for the cancer marker is isolated, for example by the methods described above, the DNA sequence can be determined, which in turn provides information regarding the amino acid sequence. Thus, if the biologically active fragment is generated by specific methods, such as tryptic digests, or if the fragment is N-terminal sequenced, the remaining amino acid sequence can be determined from the corresponding DNA sequence.

Once the amino acid sequence of the peptide is known, for example the N-terminal 20 amino acids, the fragment can be synthesized by techniques well known in the art, as exemplified by “Solid Phase Peptide Synthesis: A Practical Approach,” E. Atherton and R. C. Sheppard, IRL Press, Oxford England. Similarly, multiple fragments can be synthesized which are subsequently linked together to form larger fragments. These synthetic peptide fragments can also be made with amino acid substitutions at specific locations in order to test for agonistic and antagonistic activity in vitro and in vivo.

Polypeptid-based cancer markers can also be produced in recombinant eukaryotic or prokaryotic expression systems, and purified with column chromatography. The expression systems, include but are not limited to, E. coli, insect, or yeast expression systems.

The present invention also provides in one aspect expression vectors that carry polynucleotides of the present invention. According to a more preferred embodiment of the invention, vectors that are compatible in a yeast host are used. The example of these vectors include, for example, pJJ215, pJJ250, pJJ236, pJJ248, pJJ242 (Jones & Prakash, Yeast 6:363 (1990)); pDP6 (Fleig et al., Gene 46:237 (1986), and most preferably plasmid, pPIC9 (Invitrogen, San Diego, Calif., USA).

For the expression of a heterologous gene product in yeast preferably a vector construct is used that contains regulatory sequences capable of functioning in a methylotrophic yeast host. There are a number of methanol responsive genes in methylotrophic yeast, the expression of each being controlled by methanol responsive regulatory regions (also referred to as promoters). Any such methanol responsive promoters are suitable for use in the practice of the present invention.

Examples of specific regulatory regions include the promoter for the primary alcohol oxidase gene from Pichia pastoris AOX 1, the promoter for the secondary alcohol oxidase gene from P. pastoris AOX2 (U.S. Pat. Nos. 4,855,231, 5,032,516 and 5,166,329, incorporated herein by reference), the MOX1 gene of Hansenula polymorpha or Candida biodinii (U.S. Pat. No. 5,389,525, incorporated herein by reference), the methanol utilization genes AUG1 and AUG2 of P. methanolica, the promoter for the dihydroxyacetone synthase gene from P. pastoris (DAS), the promoter for the P40 gene from P. pastoris, the promoter for the catalase gene from P. pastoris, and the like.

Other yeast promoters include, the promoter sequence and the terminator sequence of the PGK gene, the GAP gene, the PMA gene, MF alphal promoter, galactose inducible promoters such as GAL1, GAL7 and GAL10 promoters, glycolytic enzyme promoters including TPI and PGK promoters, TRP1 promoter, CYCI promoter, CUP1 promoter, PHO5 promoter, ADH1 promoter, ADH2 promoter, GAP 491 (TDH3) and HSP promoter, and the like.

A preferred promoter region employed to drive the heterologous gene expression is derived from a methanol-regulated alcohol oxidase gene of P. pastoris. P. pastoris is known to contain two functional alcohol oxidase genes: alcohol oxidase I (AOX1) and alcohol oxidase II (AOX2) genes. The coding portions of the two AOX genes are closely homologous at both the DNA and the predicted amino acid sequence levels and share common restriction sites. The proteins expressed from the two genes have similar enzymatic properties but the promoter of the AOX1 gene is more efficient and more highly expressed; therefore, its use is preferred. The AOX1 gene, including its promoter, has been isolated and thoroughly characterized (see, Ellis et al., Mol. Cell. Biol. 5:1111 (1985) and U.S. Pat. No. 4,855,231, each of which is incorporated herein by reference in its entirety).

According to a more preferred embodiment of the invention, the AUG1 promoter is operatively linked to a nucleotide molecule encoding a heterologous peptide, and a transcriptional terminator operatively linked to this nucleotide. For expression of a desired polypeptide in methylotrophic yeast, it is preferred that the promoter and terminator are from host species genes.

The vector construct of the invention, preferably contains additional elements, such as an origin of replication, one or more selectable markers allowing amplification in alternative hosts, such as, E. coli. Selectable marker genes useful for the practice of this invention, include preferably those genes that are functional in methylotrophic yeast. For example, any gene which confers a phenotype upon methylotrophic yeast cells, thereby allowing them to be identified and selectively grown from among a vast majority of untransformed cells are intended to be encompassed within the scope of the invention. Suitable selectable marker genes include, for example, selectable marker systems composed of an auxotrophic mutant P. pastoris host strain and a wild type biosynthetic gene which complements the host's defect. For transformation of HIS₄-P. pastoris strains, for example, the S. cerevisiae or P. pastoris HIS₄ gene, or for transformation of ARG₄ mutants, the S. cerevisiae ARG₄ gene or the P. pastoris ARG₄ gene, may be employed.

In addition, the vector construct according to the invention optionally further comprises selectable marker genes that are functional in bacteria. Thus, any gene can be used which confers a phenotype on bacteria that allows transformed bacterial cells to be identified and selectively grown from among a vast majority of untransformed cells. This additional selectable marker enables DNA of the invention to be transformed into bacteria such as E. coli for amplification. Suitable selectable marker genes include the ampicillin resistance gene (Amp^{r}), tetracycline resistance gene (Tc^{r}), cycloheximide-resistance L41 gene, the gene conferring resistance to an antibiotic G418 such as the APT gene derived from a bacterial transposon Tn903, the antibiotic hygromycin B-resistance gene, and the like. When the selectable marker gene is derived from microorganisms, it is preferred to ligate it to a promoter that functions in a methylotrophic yeast to ensure the expression.

The vector constructs may further contain additional elements, such as an origin of replication and a secretory sequence. According to a preferred embodiment of the invention, the vector construct contains a DNA encoding the S. cerevisiae alpha-mating factor (AMF) pre-pro sequence (including the proteolytic processing site: lys-arg) under the regulation of a promoter region of a methanol responsive gene of a methylotrophic yeast.

The S. cerevisiae alpha-mating factor is a 13-residue peptide, secreted by cells of the “alpha” mating type, that acts on cells of the opposite “a” mating type to promote efficient conjugation between the two cell types and thereby formation of “a-alpha” diploid cells, Thomer et al., Cold Spring Harbor Laboratory 143 (1981). The AMF pre-pro sequence is a leader sequence contained in the AMF precursor molecule, and includes the lys-arg encoding sequence which is necessary for proteolytic processing and secretion (see, i.e., Brake et al., Proc. Natl. Acad. Sci. USA 81:4642 (1984)).

The transcription terminator functional in a methylotrophic yeast used in accordance with the present invention preferably has either (a) a subsegment which encodes a polyadenylation signal and polyadenylation site in the transcript, and/or (b) a subsegment which provides a transcription termination signal for transcription from the promoter used in the expression cassette.

According to one embodiment of the invention, the polynucleotide encoding an angiogenic regulatory polypeptide to be secreted and the polynucleotide encoding protein disulfide isomerase are present on a single expression vector in the host cell. This expression vector is preferably an extrachromosomal vector. More preferably the vector integrates into the chromosome of the host cell. When a single expression vector is used it is preferred that the polynucleotide encoding antiangiogenic polypeptide and the polynucleotide encoding protein disulfide isomerase are under the control of a single expression signal, for example in the form of a dicistronic operon.

According to yet another embodiment of the invention, the polynucleotide encoding the cancer marker to be secreted and the polynucleotide encoding protein disulfide isomerase are present on two mutually compatible expression vectors which are each under the control of their own promoter. According to a preferred embodiment of the invention, the expression vectors include, for example, the knock-in vector pPICZ_{b}/PDI that contains one expression cassette containing PDI; and the knock-in vectors pGAPZ_{b}/PDI/pAOX1/hESv2; and pGAPZ_{b}/PDI/pAOX1/hASv3 each containing two expression cassettes—PDI driven under the AOX1 promoter and Endostatin or Angiostatin driven under another separate AOX1 promoter, respectively. In accordance with another embodiment of this aspect of the invention, there are provided novel expression vectors comprising pPIC9K/hASv3/1, pPIC9K/hESv2/14; PIC9K/hESv2/hASv3; and pPIC9K/hESv3.

Host cells are genetically engineered to incorporate polynucleotides and express polypeptides of the present invention. Polynucleotides are introduced into host cells using techniques such as infection, transduction, transfection, and transformation. The polynucleotides are introduced alone or with other polynucleotides. Such other polynucleotides are introduced independently, co-introduced or introduced joined to the polynucleotides of the invention.

Thus, for instance, nucleotide-cancer marker of the invention are transfected into host cells with another, separate polynucleotide encoding a selectable marker, using standard techniques for co-transfection and selection in, for instance, yeast cells. In this case the polynucleotides generally are stably incorporated into the host cell genome.

Alternatively, the polynucleotides are joined to a vector containing a selectable marker for propagation in a host. The vector construct are introduced into host cells by the aforementioned techniques. The vector construct of the invention, as described and disclosed above, is capable of stable integration into the genome of the host. In particular, the vector constructs preferably integrates into the genome of the yeast, either chromosomally or extrachromosomally. The integration is more preferably chromosomal integration and is achieved, for example, through homologous recombination. If integration is desired, the DNA construct is inserted into yeast by an integration plasmid, such as, for example, pJJ215, pJJ250, pJJ236, pJJ248, pJJ242 (Jones & Prakash, Yeast 6:363 (1990)); pDP6 (Fleig et al, id.), or preferably plasmid, pPIC9 (Invitrogen, San Diego, Calif., USA). Plasmid pPIC9 contains the required regulatory sub-sequences, as well as sub-sequences encoding selectable antibiotic and/or auxotrophic markers and multiple cloning sites.

The integration of DNA into the yeast host is achieved through strategies that involve, for example, insertion or replacement methods. These methods involve strategies utilizing, for example, direct terminal repeats, inverted terminal repeats, double expression cassette knock-in, specific gene knock-in, specific gene knock-out, random chemical mutagenesis, random mutagenesis via transposon, and the like. The integration vector is, for example, flanked with homologous sequences of any non-essential yeast genes, transposon sequence or ribosomal genes. Preferably the flanking sequences are yeast protease genes or genes used as a selective marker. The DNA is then integrated on host chromosome(s) by homologous recombination occurred in the flanking sequences, by using standard techniques.

Generally, a plasmid vector is introduced as DNA in a precipitate, such as a calcium phosphate precipitate, or in a complex with a charged lipid. Electroporation is also used to introduce polynucleotides into a host. If the vector is a virus, it may be packaged in vitro or introduced into a packaging cell and the packaged virus may be transduced into cells.

Representative examples of appropriate hosts include bacterial cells, such as Escherichia coli, Bacillus subtilis and Salmonella typhimurium. Various species of Pseudomonas, Streptomyces, and Staphylococcus are also suitable hosts in this regard. Also included within the scope of the present invention are fungal cells, such as yeast cells; insect cells such as Drosophila S2 and Spodoptera Sf9 cells; animal cells such as CHO, COS and Bowes melanoma cells; and plant cells.

The heterologous gene products of the invention is preferably produced in a yeast host. The yeast host comprises Saccharomyces cerevisiae, Saccharomyces kluyveri, Schizosaccharomyces pombe, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans. Preferably the yeast is a methylotrophic yeast, i.e., yeast which is able to utilize methanol as a sole source of carbon and energy. A methylotrophic yeast strain is one that is able to use methanol as the sole carbon and energy source. Adaptation to growth on methanol is associated with induction of methanol oxidase (alcohol oxidase, AOX), dihydroxyacetone synthase (DAS), and other enzymes of methanol metabolism (Sreekrishna et al, In: Nonconventional Yeasts in Biotechnology, Springer, Berlin, page 203(1996)).

The particular methylotrophic yeast strain employed includes members of the genera Candida, Kloeckera, Saccharomyces, Rhodotorula, Hansenula, Torulopsis and Pichia (Anthony, The Biochemistry of Methylotrophs 269 (1982)). Preferred methylotrophic yeast strains are those of the genera Hansenula and Pichia. Particularly preferred methylotrophic yeast strains are Pichia pastoris and Hansenula polymorpha.

Examples of Pichia pastoris which can be employed in the present invention include Pichia pastoris GS115 (NRRL Y-15851) (U.S. Pat. No. 4,808,537), Pichia pastoris G5190 (NRRL Y-18014) (U.S. Pat. No. 4,818,700), and Pichia pastoris PPF1 (NRRL Y-18017) (U.S. Pat. No. 4,812,405), each of which is incorporated herein by reference. These auxotrophic Pichia pastoris strains are employed in the present invention in view of their ease of selection of recombinants. However, wild-type Pichia pastoris strains (i.e., NRRL Y-11430 and NRRL Y-11431) may also be employed in the present invention if such are transformed with a suitable marker gene, i.e., the SUC2 gene, such that the strains are capable of growth on sucrose, or with an antibiotic resistance gene, such as the Kanamycin gene which confers resistance to G418.

Pichia pastoris strain GS 115 is the most preferred methylotrophic yeast strain employed in the present invention. Pichia pastoris clones containing endostatin and angiostatin genes are disclosed in the U.S. Provisional application No. 60/361,353 to Liang Hong et al., filed Mar. 5, 2002, content of which is incorporated herein by reference in its entirety.

In a particularly preferred embodiment of this aspect of the invention, the Pichia pastoris clones comprise EM6688; EM6688.2; EM6688.3; EM6688.6; EM6688/pGAPZB/PDI; EM6688.2/pGAPZ_{b}/PDI; EM6688.3/pGAPZB/PDI; EM6688.6/pGAPBlastB/PDI; EM6688/pPICZ_{b}/PDI; EM6688.2/pPICZ_{b}/PDI; X33/pGAPZ_{b}/PDI/pAOX₁/hESV₂; EM6688/GAPZ_{b}/PDI/pAOX₁/hESV₂; EM6688/pAOX/PDI/pAOX/hESv2; EMAN98.3; EMAN98/pGAPZB/PDI; X33/pGAP/PDI/pAOX/hASv3; EMAN98/pGAP/PD1/pAOX/hASv3; and EMAN98/pPICZ/PDI.

The vector constructs are introduced, for example, into essentially pure cultures of methylotrophic yeast cells by, transforming spheroplasts that have been produced by enzymatic digestion of the cell walls. The transforming DNA is incubated in the presence of calcium ions and polyethylene glycol, then the cells walls are regenerated in selective growth medium (See, i.e., Stroman et al., U.S. Pat. No. 4,879,231, incorporated herein by reference.) Transformation of whole cells of methylotrophic species of the genus Pichia in buffered solutions of lithium chloride or lithium sulfate are described in Cregg et al., U.S. Pat. No. 4,949,555, incorporated herein by reference.

According to a more preferred embodiment of the invention, the transformation is achieved by an electroporation apparatus (MaxCyte Rockville, Md.). The specific method of electroporation along with the high efficiency electroporation apparatus continuously provided superior transformation efficiency with respect to a number of different host cells and DNA expression vector employed.

Positive transformants are characterized by Southern blot analysis (see, Maniatis et al., Cold Spring Harbor Laboratory Press (1982)), and methanol-responsive heterologous RNA transcription and translation products are detected by Northern blot and Western blot, respectively. Host cells containing vector constructs of the present invention, as selected above, are then cultured to produce recombinant heterologous gene product.

Following transformation of a suitable host strain, the host strain was grown to an appropriate cell density. The engineered host cells were cultured in conventional nutrient media, which were modified as appropriate for, inter alia, activating promoters, selecting transformants or amplifying genes. Culture conditions, such as, pH, temperature during the induction phase, macro-micro nutrients, and methanol feed rate, were modified according to the particular expression system used and the expressed polypeptide sought. Where the selected promoter is inducible, it was induced by appropriate means (i.e., temperature shift or exposure to chemical inducer) and cells are cultured for an additional period. Cells typically then are harvested by centrifugation, disrupted by physical or chemical means, and the resulting crude extract retained for further purification.

In accordance with one embodiment of the invention, a methylotrophic yeast was cultured on a minimal defined medium with an excess of non-inducing carbon source (i.e., glycerol). The growth medium generally selects for cells containing the vector construct by, for example, drug selection or deficiency in an essential nutrient that is complemented by the selectable marker on the vector.

Expression of the heterologous gene product was typically induced by limiting the non-inducing carbon source and preferably by adding the inducing carbon source, i.e., methanol, so as to derepress the methanol responsive promoter. Transformed cells that were particularly well suited (especially those exhibiting high and stable expression levels) for expression of the heterologous gene product were selected, and then subcultured.

According to an embodiment of the invention, transformed strains that were of the desired phenotype and genotype were grown in fermentors. For example, in one embodiment, for the large-scale production of the heterologous gene product in methylotrophic yeast, a three-stage, high cell-density, fed batch fermentation system was used. In the first stage, or growth stage, expression hosts were cultured in defined minimal medium with an excess of a non-inducing carbon source (i.e., glycerol). When grown on such carbon sources heterologous gene expression was completely repressed, which repression allows the generation of cell mass in the absence of heterologous protein expression. It is presently preferred that during this growth stage the pH of the medium is maintained at about 5. Next, a short period of non-inducing carbon source limitation growth is allowed to further increase cell mass and derepress the methanol responsive promoter. The pH of the medium during this limitation growth period was adjusted to the pH value to be maintained during the production phase, which is generally carried out below a pH of about 4; preferably at a pH in the range of about 2-3.5. Subsequent to the period of growth under limiting conditions, methanol alone (referred to herein as “methanol excess fed-batch mode”) or a limiting amount of a non-inducing carbon source plus and methanol (referred to herein as “mixed-feed fed-batch mode”) are added in the fermentor, inducing the expression of the heterologous gene driven by a methanol responsive promoter. This third stage is the so-called production stage.

The heterologous gene product is secreted intercellular or extracellular. According to a preferred embodiment of the invention, the heretrologous gene product is expressed extracellular. For secretion, a signal sequence may be supplied, i.e., the S. cerevisiae prepro alpha mating factor (MF alpha. prepro) leader sequence, as described in, i.e., U.S. Pat. No. 5,324,639 and Vedvick et al., J. Ind. Microbiol. 7:197-201 (1991) both of which are incorporated herein by reference.

To extract the heterologous gene product form the yeast, cells are disrupted. The cells are, for example, milled by typically using glass beads, or lysed, usually while keeping the cells chilled, i.e., at or below about 4-7° C. An extraction buffer adjusted to about pH 7.0 to 7.2 is employed that preferably contains protease inhibitors; reducing agents such as dithiothreitol or 2-mercaptoethanol; and a detergent, particularly a non-ionic detergent such as TRITON X-114 (polyethylene glycol tertiary octylphenyl ether), TRITON X-100 (polyethylene glycol mono [p-(1,1,3,3-tetramethyl-butyl) phenyl] ether).

Methods of isolation and purification of the expressed heterologous gene product can be achieved by conventional chemical purification means, such as liquid chromatography, immunoaffmity chromatography, lectin affinity chromatography, gradient centrifugation, and gel electrophoresis, among others. Methods of protein purification are generally described in, for example, Scopes R., Protein Purification (1982), and for P. pastoris a general purification protocol is described in, for example, Clegg et al., Bio/Technol. 11:905-910 (1993), each of which is incorporated herein by reference. It is preferred that a reducing agent such as DTT or the like, a non-ionic detergent, and a phosphate buffering agent be present throughout most stages of the purification process to maintain stability of the purified heterologous gene product.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

Effect of PSA on bFGF-Induced Proliferation of HUVE Cells

Proliferation assays familiar to those skilled in the art using human umbilical vein endothelial (HUVE) cells were used to determine the effect of PSA on bFGF-induced proliferation of human umbilical vein endothelial cells.

The materials for this experiment included HUVE cells and media for their proliferation, Endothelial Cell Basal Medium (EBM) and Endothelial Cell Growth Medium (EGM) (Clonetics, San Diego, Calif.). Also used was Human Prostate-Specific Antigen (Vitro Diagnostics, Inc., Littleton, Colo., catalog number 4-70-455).

The proliferation assay involved the routine culturing of HUVE cells to confluency in EGM media. The cells were trypsinized and plated in a 96-well plate at 5000 cells per well per 100 mL EBM media. The cells were plated in EBM for 24 hours. Next bFGF at 5 ng/ml and PSA at various concentrations were added to the wells (1-100 μg/ml). The cells were cultured for 72 hours after which cell proliferation was determined using a standard bromo-uridine incorporation method.

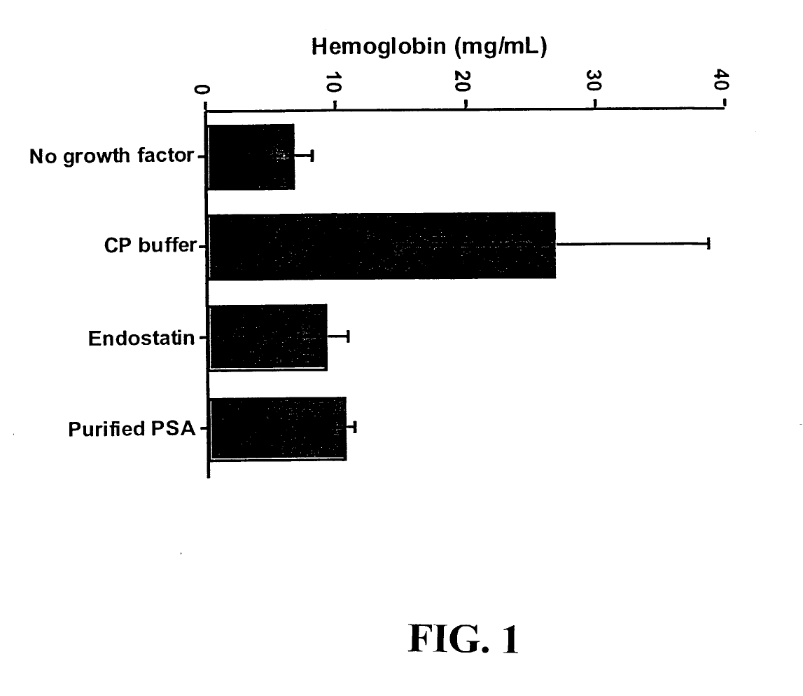

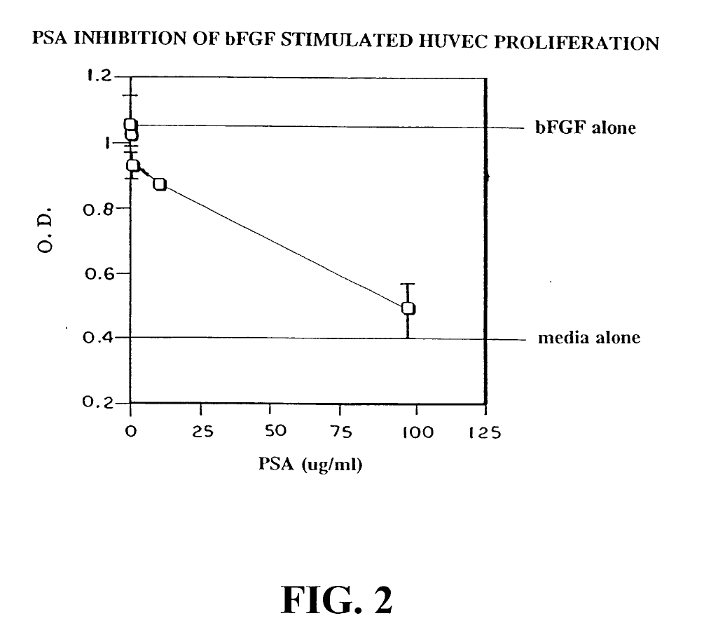

PSA inhibited bFGF-induced proliferation of HUVE cells in a dose dependent manner in two different experiments. The relative inhibitory effects of the various concentrations of PSA are shown graphically in FIGS. 1, 2 and 4 respectively.

### Example 2

Effect of PSA on bFGF-Induced Proliferation of BCE Cells

Proliferation assays familiar to those skilled in the art using bovine capillary endothelial cells (BCE) were used to determine the effect of PSA on bFGF-induced proliferation of BCE Cells.

The materials for this experiment included BCE cells and media for their proliferation, Endothelial Cell Basal Medium (EBM) and Endothelial Cell Growth Medium (EGM), (Clonetics, San Diego, Calif.). Also used was Human Prostate-Specific Antigen, (Vitro Diagnostics, Inc., Littleton, Colo., catalog number 4-70-455).

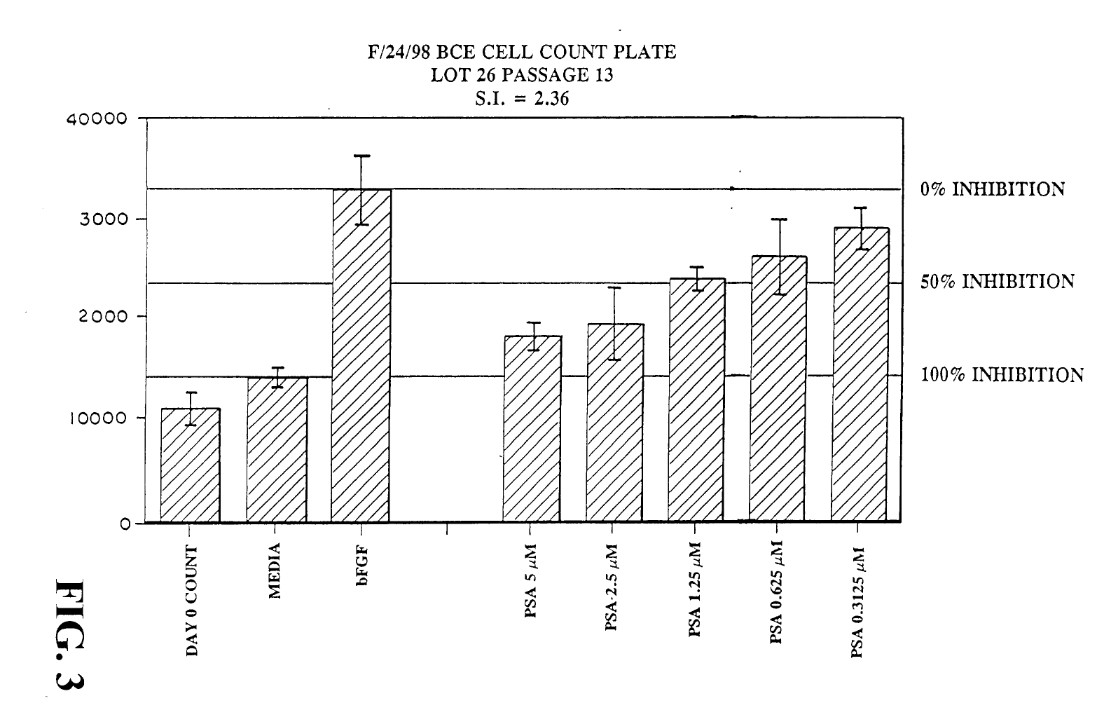

The cells were cultured for 72 hours after stimulation with bFGF in the presence or absence of PSA at various concentrations as indicated in FIG. 3.

PSA inhibited bFGF-induced proliferation of BCE cells in a dose dependent manner. The relative inhibitory effects of the various concentrations of PSA are shown graphically in FIGS. 3 and 5.

### Example 3

In vivo Effect of PSA on Tumor Growth

PSA (Vitro Diagnostics, Inc., Littleton, Colo., catalog number 4-70-455) was used to treat mice that had been inoculated with B16BL6 Melanoma. The mice were inoculated with 5×10⁴ tumor cells intraveneously on day 0. On day 3 and for the next consecutive 11 days, the animals were treated with PBS or 30 μg of (a) PSA 9 μM, or (b) a control protein 15 μM, or (c) ENDOSTATIN™ as a positive control 15 μM. The mice were sacrificed at day 14 and the lung metastases were counted. The mean number of lung metastases for each of the treated groups was compared with the PBS control to give a T/C (treated/control) ratio.

As summarized below, mice receiving a PSA treatment had a significantly lower occurrence of lung metastases as compared to control mice. PSA demonstrated modest growth inhibitory effects on tumor lesions in mouse lungs (20 and 40% inhibition).

Effect of PSA on Metastatic (B16B16) Disease in Mice

| | | Mean Lung Metastases T/C: | | |
|---|---|---|---|---|
| Treatment | Dose: | ±1S.D. of the Mean: | | p value |
| PBS | 0.1 ml | 115 ± 16 | 1.0 | — |
| PSA | 9 μM | 70 ± 8 | 0.61 | 0.003 |
| Negative | 15 μM | 88 ± 10 | 0.77 | 0.044 |
| Control | | | | |
| Endostatin ™ | 15 μM | 16 ± 8 | 0.14 | 0.0002 |
| Protein | | | | |

### Example 4

Antiproliferative Effects of PSA

The antiproliferative effects of PSA were demonstrated in Human Umbilical Vein Endothelial Cells (HUVEC).

Human umbilical vein endothelial cells (HUVEC): Single donor HUVEC were obtained frozen at passage 1 from Clonetics (San Diego, Calif.). The cells were maintained in endothelial cell growth medium (EGM, Clonetics) supplemented with bovine brain extract (Clonetics). Cells were 2 cultured on 75 cm vented tissue culture flasks (Costar Corning, N.Y.) at 37° C., in moist air containing 5% CO₂. HUVEC were used at passages 2-5 in all following examples. For proliferation assays HUVEC were obtained from trypsin/versene (Biowhittaker, Walkersville, Md.) digested monolayers. Cells were resuspended in endothelial cell basal medium-2 (EBM-2, Clonetics) supplemented with 2% heat inactivated FBS (Hyclone, Logan, UT) and 2 mM L-glutamine (Biowhittaker). Two hundred μL of HUVEC at 2.5×104/mL were plated into 96 well flat bottom plates (Costar) and incubated overnight at 37° C. in 5% CO₂. These cultures were then washed and exposed to various concentrations of purified human PSA (Vitro Diagnostics, Littleton, Colo.) or to media alone in a total volume of 100 μL and incubated for 30 minutes at 37° C. in 5% CO₂. After 30 minutes of incubation, an additional 100 μL of assay media containing 10 ng/mL of FGF-2 (R&D Systems, Minneapolis, Minn.) was added to all cultures except for the control which contained media alone. All cultures were incubated for an additional 48 h at 37° C. in 5% CO₂. Cell proliferation was assessed with a calorimetric ELISA kit (Boehringer Mannheim, Indianapolis, Ind.) that measured the amount of BrdU incorporated during DNA Synthesis. Results are expressed as the mean absorbance of triplicate cultures measured at 370 nm (reference wavelength 492 nm).

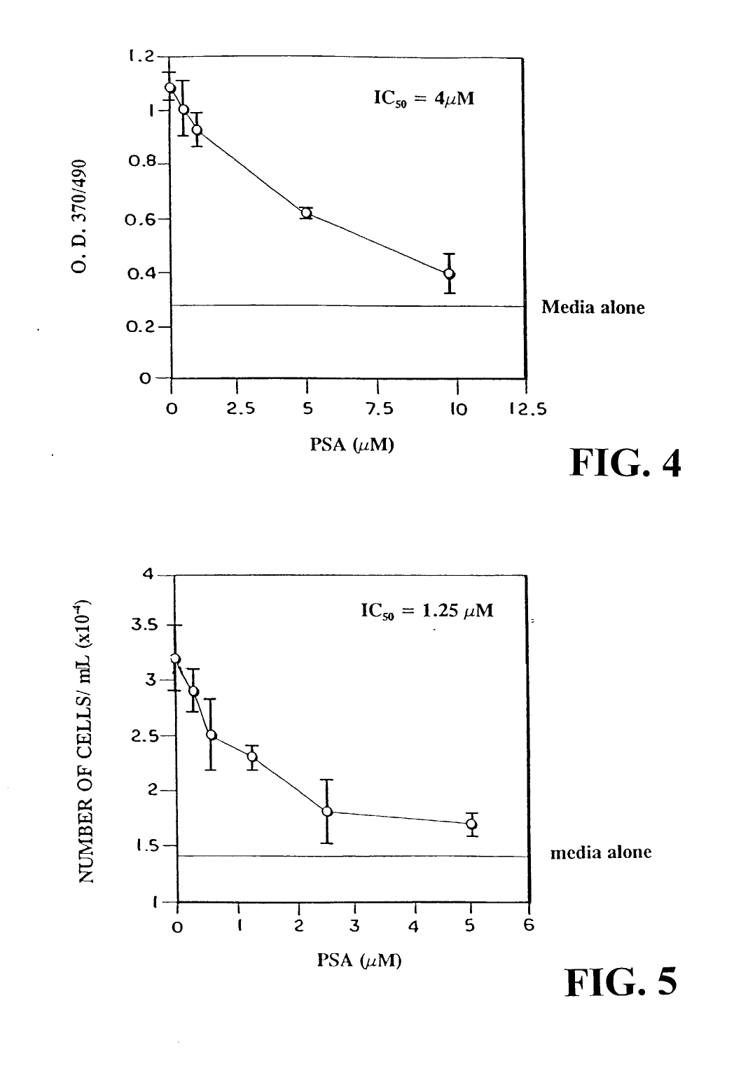

As shown in FIG. 4, purified human PSA demonstrated a potent and dose-related inhibitory activity on FGF-2-stimulated proliferation of HUVEC cells with an IC₅₀ of 4 μM.

### Example 5

Antiproliferative Effects of PSA on Cells Other Than HUVECs

To determine if PSA inhibited a variety of endothelial cells or simply displayed specificity for HUVECs, the ability of PSA to inhibit bovine adrenal cortex endothelial cell (BCE) and human microvascular dermal cell (HMVEC-d) proliferation was also evaluated (see, FIGS. 5 and 6).

BCE were obtained at passage 9 as a generous gift from Dr. J. Folkman (Children's Hospital, Harvard Medical School, Boston, Mass.). The cells were cultured and maintained as described by O'Reilly et al., Cell 79:315 (1994). For evaluation of PSA ability to inhibit BCE proliferation, assays were performed also as described by O'Reilly et al (1994) id., and cells were exposed to various concentrations of purified PSA or media alone for 30 minutes at 37° C. in 10% CO₂ prior to stimulation with FGF-2. Cell proliferation was assessed by counting the number of cells with a Coulter Z 1 particle counter (Coulter Corp., Hialeah, Fla.). Results were expressed as the mean number of cells counted in triplicate culture wells.

Single donor adult HMVEC-d were obtained frozen at passage 4 from Clonetics. The cells were maintained in microvascular endothelial cell growth medium-2 (EGM-2-MV, Clonetics). Cells were cultured on 75 cm² vented tissue culture flasks at 37° C., in moist air containing 5% CO₂. HMVEC-d were used at passages 5-8 in all experiments. For proliferation assays HMVEC-d were obtained from trypsin/versene (Biowhittaker) digested monolayers. HMVEC-d were resuspended in endothelial cell basal medium-2 (EBM-2, Clonetics) supplemented with 2% heat inactivated FBS (Hyclone) and 2 mM L-glutamine. Cells at 1.6×104/ml were plated into 1.5% gelatin coated 24 well flat bottom plates (Costar) and incubated overnight at 37° C. in 5% CO₂. These cultures were then washed and exposed to various concentrations of purified PSA or to media alone and incubated for 30 minutes at 37° C. in 5% CO₂. After 30 minutes FGF-2 at 10 ng/mL was added to all cultures except for the control which contained media alone. All cultures were incubated for an additional 48 h at 37° C. in 5% CO₂. Cell proliferation was assessed by counting the number of cells/well with a Coulter Z 1 particle counter (Coulter Corp). Results are expressed as the mean number of cells counted in triplicate culture wells.

As shown in FIGS. 5 and 6, PSA potently inhibited FGF-2-stimulated endothelial cell proliferation, with an IC₅₀ for BCE cells of 1.0 μM, and an IC₅₀ for HMVEC-d of 0.6 μM. Accordingly, inventors demonstrated that the antiproliferative effects of PSA were not limited to, or specific for, HUVECs.

### Example 6

Specificity of Anti-Proliferative Effects of PSA

In order to demonstrate that the antiproliferative effects of PSA are specific for endothelial cells, the inventors conducted experiments to evaluate direct stimulatory or inhibitory effect on the proliferation of cancer cells.

B16BL6, a murine melanoma, obtained from the NCI-FCRC cell repository were maintained in DMEM (Biowhittaker), supplemented with 5% heat inactivated fetal bovine serum FBS (Hyclone) and 2 mM L-glutamine. Tumor cells were cultured on 75 cm² vented tissue culture flasks at 37° C., 5% CO₂ in moist air. For proliferation assays B16BL6 were obtained from trypsin/versene (Biowhittaker) digested monolayers. B16BL6 at 1.25×104/ml were plated into 96 well flat bottom plates (Costar) and incubated overnight at 37° C. in 5% CO₂. These cultures were then washed and exposed to various concentrations of purified PSA or media alone and incubated for an additional 48 h at 37° C. in 5% CO₂. Tumor lines showed FGF-2 independent growth in vitro. Cell proliferation was assessed with a calorimetric ELISA kit (Boehringer Mannheim) for BrdU incorporation. Results are expressed as the mean absorbance of triplicate cultures measured at 370 nm (reference wavelength 492 μm).

Human prostate cancer cell line, PC3, also a kind gift from Dr. Folkman, was used to determine PSA inhibitory effects on FGF-2 independent cell growth. PC3 were obtained by gentle removal of cells from the tissue culture flask with a cell scraper (Costar). Cells were resuspended in DMEM supplemented with 10% heat inactivated FBS and 2 mM L-glutamine, plated into 24 well flat bottom plates at 6×104/mL (Costar) and incubated overnight at 37° C. in 5% CO₂. These cultures were then washed and exposed to various concentrations of purified PSA or to media alone (no FGF-2 added to the cultures) and incubated for 30 minutes at 37° C. in 5% CO₂. After 30 minutes of incubation, additional assay media was added to all wells. All cultures were incubated for an additional 48 hours at 37° C. in 5% CO₂. Cell proliferation was assessed by counting the number of cells with a Coulter Z1 particle counter. Results are expressed as the mean number of cells in triplicate cultures.

As shown in the figures, the growth of murine melanoma cells (B16BL6) or human prostate cancer cells (PC3) was unaffected by the addition of purified human PSA (see FIGS. 7 and 8, respectively).

### Example 7

Anti-Migratory Effects of PSA on Endothelial Cells

In order to evaluate the in vitro effects of PSA on endothelial cell migration in response to FGF-2 or VEGF, confluent monolayers of HUVEC were scraped to remove a section of monolayer and cultured for 24 hr with FGF-2 or VEGF in the presence or absence of purified human PSA.

A wound migration assay was performed as described by Kubota et al. J. Cell Biol. 107:1589 (1988) to determine the ability of PSA to block HUVEC migration induced by recombinant FGF-2 or recombinant VEGF 165 (R&D Systems). Briefly, 5×10⁵ HUVEC in EGM were plated onto 1.5% gelatin coated 60 mm tissue culture dishes (Corning) and incubated for 72 h at 37° C. in 5% CO₂ in moist air. After incubation, confluent monolayers were wounded with a sterile single edged No. 9 razor blade (VWR Scientific, Media, Pa.) which resulted in a straight edge that separates the confluent area from the denuded area. Immediately after monolayers were wounded, the cells were washed with PBS (Biowhittaker) to remove cellular debris, and further incubated in EBM supplemented with 1% heat inactivated FBS, 2 mM L-glutamine, 100μ/ml penicillin, 100 μg/ml streptomycin and 0.25 μg/ml fungizone. The monolayers were exposed to 2 ng/mL of FGF-2 or to 10 ng/mL VEGF in the presence or absence of different concentrations of PSA (Vitro Diagnostics), or to media alone for 16-20 h in 5% CO₂ in moist air. The monolayers were fixed with absolute methanol and stained with Hematoxylin Solution, Gill No.3 (Sigma Diagnostics, St. Louis, Mo.). Migration was quantified by counting the number of cells that migrated from the wound edge into the denuded area. Cells were counted at 200× magnification using an inverted light microscope with an ocular micrometer along a 1 cm distance. The values represent the mean number of cells in duplicate cultures.

As shown in FIGS. 9 and 10, PSA exerted dose-response inhibitory effects on FGF-2 and VEGF-stimulated migration, respectively, with an IC₅₀ for PSA versus FGF-2 of 1.2 μM, and versus VEGF of 4 μM (FIGS. 9 and 10, respectively).

### Example 8

Effect of PSA on Invasion by Endothelial Cells

Assays to measure migration of endothelial cells were coupled with another parameter of angiogenesis, invasion, by performing the assay in a two-chamber environment where the chambers are separated with a membrane filter coated with matrigel. In this assay, PSA, at 5 μM, inhibited FGF-2-stimulated HUVEC invasion through matrigel by 77%. In addition, at concentrations ranging from 0.3 μM to 31M purified human PSA inhibited tube formation of HWVEC in matrigel by approximately 50%.

Biocoat matrigel 8 μm invasion chambers (Collaborative Biomedical Products, Bedford, Mass.) were pre-coated with 38 μg of matrigel (Collaborative Biomedical Products). Chambers were rehydrated with warm (37° C.) EBM supplemented with 1% heat inactivated FBS and 2 mM L-glutamine for 2 h at room temperature. After rehydration, the media was gently removed and replaced immediately with 5×10⁴ HUVEC pretreated with PSA (5 μM) or with media alone for 30 minutes at 37° C. in 5% CO₂. The lower chambers were filled with assay media supplemented with 5 ng/mL of FGF-2 or assay media alone. These chambers were then incubated for 24 h at 37° C. in 5% CO₂. After incubation, the non-invading cells were removed by scrubbing the inserts with a cotton swab. The cells on the lower surface of the membrane were stained with Diff-Quik (Dade Diagnostics, Aquado, PR). The membrane was removed and mounted on a microscope slide. The number of cells invaded was determined by counting the cells in the central field of the membrane of triplicate cultures within a 24×36 mm ocular grid at 150× magnification.

Matrigel obtained from Collaborative Biomedical Products (Bedford, Mass.) exists as a liquid below 4° C. and forms a gel at temperatures above 4° C. For induction of endothelial tube formation the following procedure was adapted from the protocol of Kubota et al., J. Cell Biol. 107:1589 (1988). Briefly, matrigel is aliquoted into a 96 well tissue culture plate (Costar) in a volume of 65 μL. The plate is incubated for 30 min at 37° C. to allow the matrigel to gel. Following incubation, various doses of PSA (Vitro Diagnostics) were added to the matrigel in a volume of 100 μL. Included as a positive control was 2-methoxyestradiol (Fotsis Nature and media alone served as negative control. The HUVECs were harvested and adjusted to 1×10⁵ cells/ml in EGM supplemented with 5% heat inactivated FBS. One hundred μL cell suspension was added to the wells and incubated at 37° C., 5% CO₂ in moist air. After 4 hours of incubation, endothelial cells elongate and tube structures begin to form by 16 hrs endothelial cells are microscopically evaluated for tube formation.

The results of this experiment demonstrated that inhibition appeared to be dose dependent and not the result of toxicity. Endothelial cells appeared viable and some elongation was noted but, there were no junctions made by the endothelial cells.

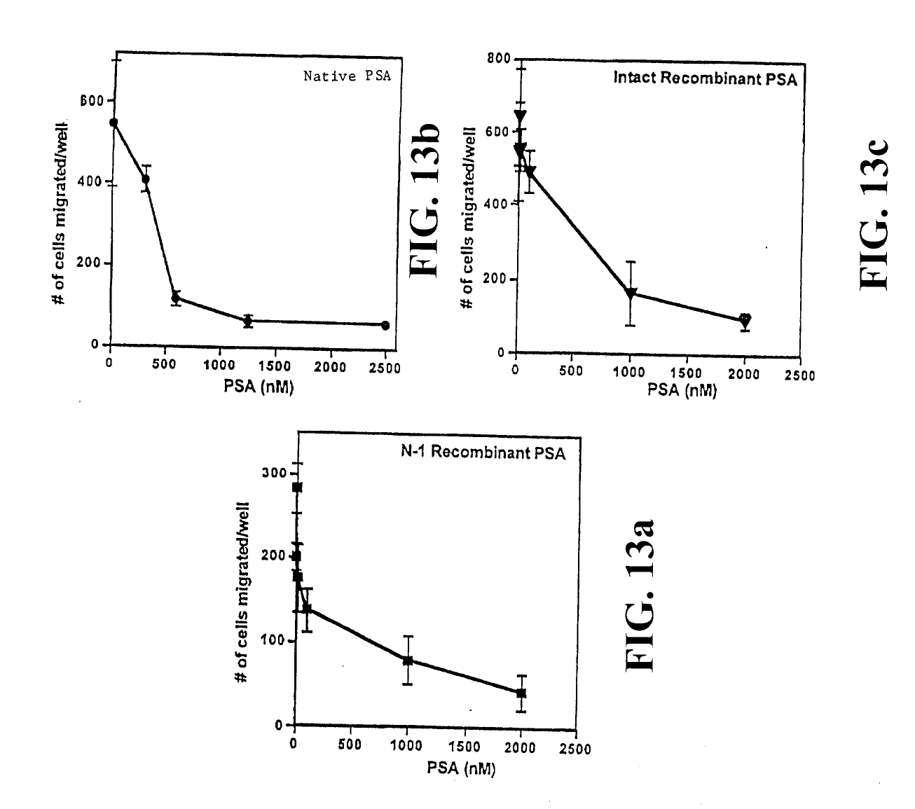

Effect of native PSA and recombinant PSAs (intact and N-1 variant) on VEGF-induced migration of HUVEC. HUVEC migration induced by 5 ng/ml of VEGF was evaluated in a modified Boyden chamber as shown in FIG. 13. Cells were pre-incubated for 30 minutes in the presence or absence of PSA and were then allowed to migrate through an 8 micron polycarbonate PVP-free filter coated with collagen type 1 for 6 h. The non-migrated cells were removed, and the filter was fixed and stained with Diff-Quik. The number of migrated cells was determined using the Image-Pro plus analysis system.

The results regarding in vivo antiangiogenic activity of PSA were obtained by using a matrigel assay. In these experiments, a matrigel plug is loaded with FGF-2 and implanted under the skin of normal mice. Typically, matrigel was supplemented with 2 mg/ml of FGF-2 and was then injected subcutaneously under the skin of male C57BL6/J mice. Animals were treated subcutaneously with native PSA (100 μg/day) or, for comparison Endostatin™ protein (5 mg/kg/day) for 5 consecutive days. After 5 days animals are sacrificed and the plugs are removed and homogenized. The concentration of hemoglobin was measured as an assessment of angiogenic responsiveness. The results of this experiment is demonstrated in FIG. 1.

### Example 9

Effect of PSA Serine Protease Activity on Angiogenesis

PSA has serine protease activity, and in serum, PSA is predominantly bound to the protease inhibitor, alpha-I anti-chymotrypsin (ACT)(Lilia et al. Clin. Chem. 37:9 (1991)). The ability of ACT to inhibit both serine protease activity of purified PSA as well as the antimigratory effects of PSA on FGF-2-stimulated HUVEC was tested as described below.

The ability of alpha-1 antichymotrypsin to inhibit the proteolytic activity of PSA was measured using the synthetic substrate S-2586 (MeO-Suc-Arg-Pro-Tyr-NH-Np). The rate of hydrolysis of S-2586 (1.3 mM) by PSA 6 μg (0.89 μM) with and without pretreatment for 4 hours at 37° C. with an equimolar concentration of ACT (Sigma Chemical Co., St. Louis, Mo.) was monitored at 405 nm in 50 mM Tris/HCl, pH 7.8 containing 0.1 M NaCl. Stable complexes of PSA and ACT formed after 4 hours of incubation and were confirmed by SDS-PAGE. The results were plotted as an increase in absorbance vs. time in minutes. The ability of ACT (Sigma) to inhibit the anti-migratory activity of PSA was measured by preincubating PSA (5 μM) with an equimolar concentration of ACT for 4 h at 37° C. prior to addition to the HUVEC migration assay.

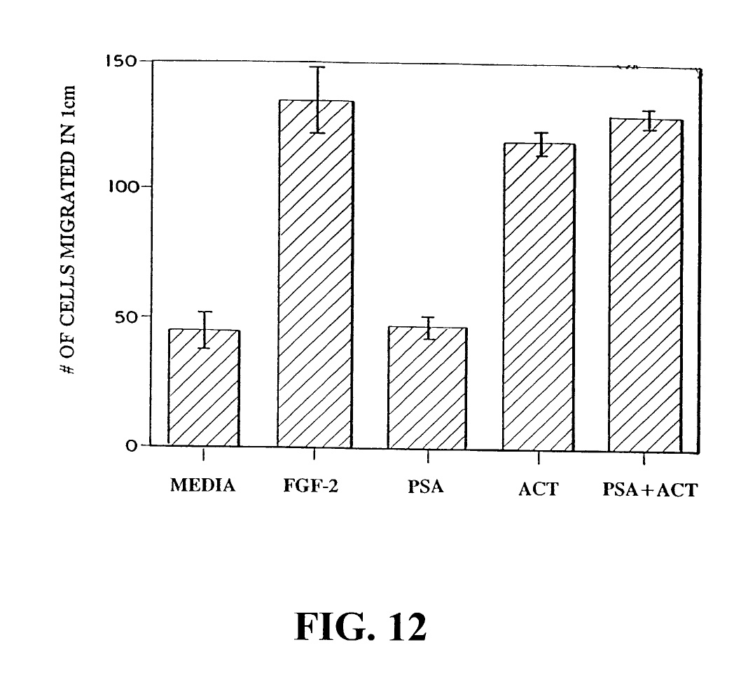

As shown in FIGS. 11 and 12, using equimolar concentrations of ACT and PSA, preincubation of PSA with ACT blocked both serine protease activity of purified PSA (FIG. 11) as well as the antimigratory effects of PSA on FGF-2-stimulated HUVEC (FIG. 12). Accordingly, these results demonstrate that the antiangiogenic properties of PSA are related to its serine protease activity.

### Example 10

Anti-Migratory Effect of CEA on Endothelial Cells

In order to evaluate the in vitro effects of CEA on endothelial cell migration in response to VEGF, confluent monolayers of HUVEC were scraped to remove a section of monolayer and cultured for 24 hr with VEGF in the presence or absence of recombinant human CEA. CEA used was a Recombinant Human Carcinoembryonic Antigen (rCEA)—affinity pure-source: Human breast carcinoma MCF-7 cell line, Vitro Diagnostic, Inc. Lot# 990525D1AA.

A wound migration assay was performed as described by Kubota et al., id. to determine the ability of CEA to block HUVEC migration induced by recombinant VEGF 165 (R&D Systems). Briefly, 5×10⁵HUVEC in EGM were plated onto 1.5% gelatin coated 60 mm tissue culture dishes (Corning) and incubated for 72 h at 37° C. in 5% CO₂ in moist air. After incubation, confluent monolayers were wounded with a sterile single edged No. 9 razor blade (VWR Scientific, Media, Pa.) which resulted in a straight edge that separates the confluent area from the denuded area. Immediately after monolayers were wounded, the cells were washed with PBS (Biowhittaker) to remove cellular debris, and further incubated in EBM supplemented with 1% heat inactivated FBS, 2 mM L-glutamine, 100 μL/ml penicillin, 100 μg/ml streptomycin and 0.25 μg/ml fungizone. The monolayers were exposed to 5 ng/ml VEGF in the presence or absence of different concentrations of CEA (Vitro Diagnostics), or to media alone for 16-20 h in 5% CO₂ in moist air. The monolayers were fixed with absolute methanol and stained with Hematoxylin Solution, Gill No.3 (Sigma Diagnostics, St. Louis, Mo.). Migration was quantified by counting the number of cells that migrated from the wound edge into the denuded area. Cells were counted at 200× magnification using an inverted light microscope with an ocular micrometer along a 1 cm distance. The values represent the mean number of cells in duplicate cultures.

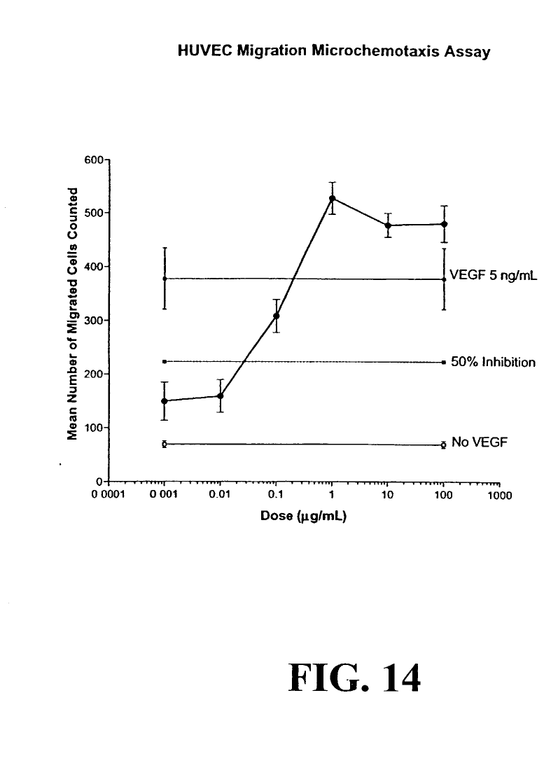

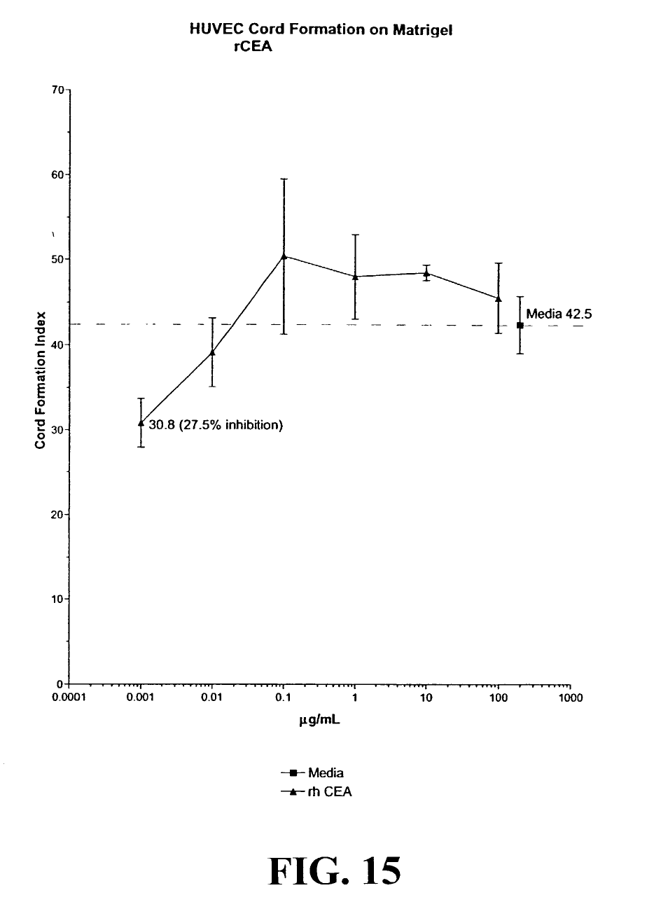

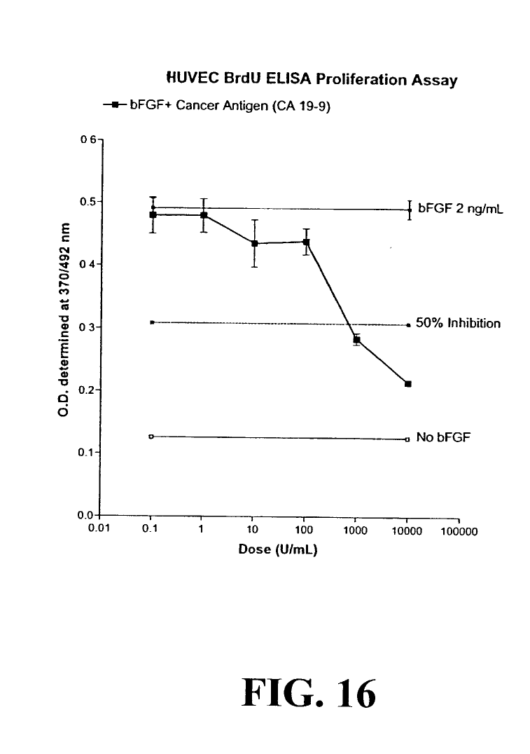

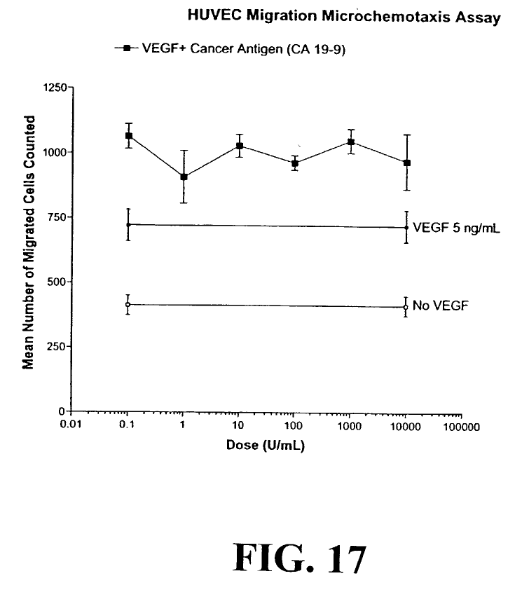

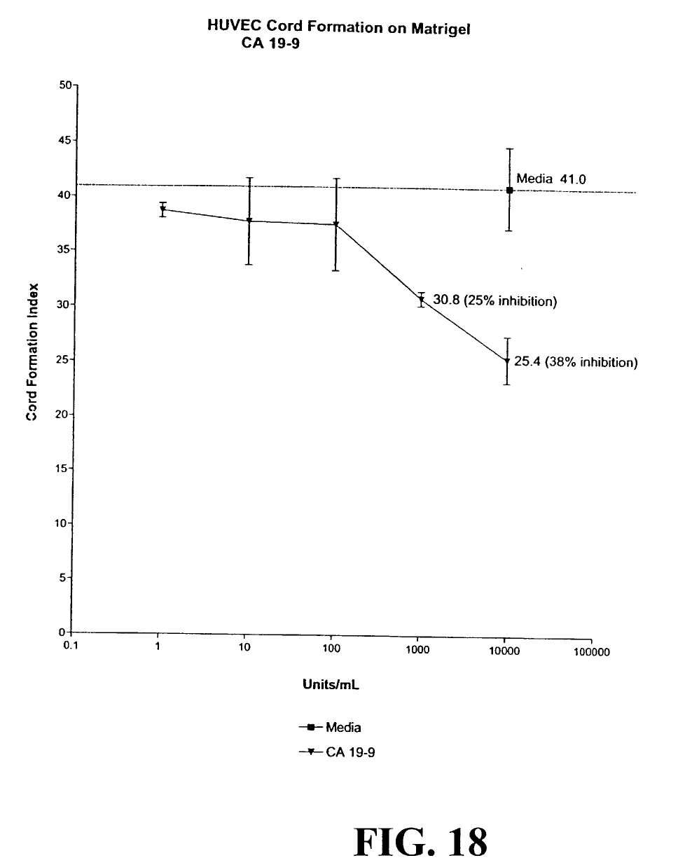

As shown in FIG. 14, CEA exerted a dose-response inhibitory effect on VEGF-stimulated migration, with an IC₅₀ for CEA versus VEGF of approximately 20 ng/ml. Recombinant CEA (rCEA) inhibited VEGF-induced migration of HUVEC with an IC₅₀ between 10 and 100 ng/ml. Higher concentrations from 1-100 μg/ml are stimulatory. In addition, concentration of 1 ng/ml of rCEA inhibited HUVEC cord formation on matrigel by 28% while higher concentrations of 1-100 μg/ml enhanced cord formation. Recombinant CEA had no effect on FGF-2-stimulated HUVEC proliferation.

### Example 11

Anti-Proliferation Effect of CA 19-9 On Endothelial Cells

Cancer antigen (CA 19-9), derived from human ascites fluid, (Aspen Bio, Inc. Lot# M1001F181S) was used in bioassays in order to determine its antiangiogenic potential. The results of the bioassays demonstrated that CA 19-9 at an IC₅₀ of 1000 U/ml inhibited FGF-2-stimulated HUVEC proliferation. The addition of 10,000 U/ml blocked cord formation by 38%. Concentrations of 1000 U/ml inhibited cord formation by 25%. No significant differences were observed with doses of 100 U/ml or less. CA19-9 had no effect on VEGF-stimulated migration or on the ability to induce apoptosis in HUVEC.

### Example 12

Inhibitory Effect of HCG on Formation and Proliferation of Endothelial Cells

Cancer marker human chorionic gonadotrophin (HCG), including HCG-α and HCG-β were used in bioassays to determine their antiangiogenic effect. The result of these bioassays are demonstrated in FIGS. 19-24. The results demonstrated HCG-β subunit inhibited FGF-2 HUVEC proliferation with an IC₅₀ between 10 and 100 μg/ml. The concentration of HCG-A that resulted in 50% inhibition of VEGF stimulated HUVEC migration was 100 μg/ml. HCG-α did not block the ability of HUVEC to form cords on matrigel. HCG-β subunit exhibited no effect on FGF-2 proliferation of HUVEC. At concentrations, ranging from 1 to 100 μg/ml, HCG-β inhibited VEGF-stimulated migration by 50% at 1 μg/ml. At this same concentration (1 μg/ml) HCG-p was able to repeatedly inhibit cord formation by approximately 28%, which was statistically different from media control (p=0.02). All other doses were not statistically different from each other. HCG-p was able to induce apoptosis of HUVEC in a dose-dependent fashion. A 13% induction of apoptosis was observed with a concentration of 10 μg/ml while 4% was observed after treatment with 1 μg/ml.

### Example 13

Inhibitory Effect of NSE on Endothelial Cell Proliferation

Neuron Specific Enolase (NSE), derived from human brain (Lee Scientific, Inc. Lot# F88705) was used in several bioassays in order to determine its antiangiogenic potential. NSE inhibited FGF-2-stimulated HUVEC proliferation with an IC₅₀ of 20 μg/ml. NSE did not inhibit VEGF-stimulated migration of HUVEC or block the ability of HUVEC to form cords on matrigel. NSE did not induce apoptosis in HUVEC as measured by Tunel.

### Example 14

Pre-Clinical Studies: In vitro, Maintenance of HUVEC from Cascade Biologics

Human Umbilical Vein Endothelial Cells (HUVEC) were received frozen at passage 1 from Cascade logics, Inc., Portland, Oreg. (800-778-477)(Cat. # C-003-5C single donor). Endothelial cell Growth Medium (M-200, Cascade Cat. M-200-500) was supplemented with LSGS (Cascade Cat. # S-003-3K) and stored at −20° C., with 1% L-Glutamine (BioWhittaker Cat.# 17605-E). In general, cells were split 1:6 once a week, (80% Confluent) using 75 cm² tissue culture flasks (Costar Cat. # 3376). Cells were incubated at 37° C. in 5% CO₂ and were obtained from the flask by trypsinization. Culture media was removed and cells were washed with 5 ml PBS (Biowhittaker 17-516F). Two mls of trypsin-versene (Biowhittaker Cat. # 17-161E) were added to the cells. Flaks were rocked to ensure that the entire surface is covered at room temperature for 1 minute. Cells were neutralized with an excess of warm medium, trypsin-versene/media was removed by centrifugation at 1000 rpm for 5 minutes, cells were then resuspended in 6 ml complete growth media.

### Example 15

Pre-Clinical Studies: Protocol: In vitro, BrdU Proliferation, HUVEC

HUVEC was harvested from flask by trypsinization (see HUVEC Cascade Maintenance Protocol). Cells were resuspended at 2.5×10⁴/ml in M-200, containing 2% heat inactivated FBS with 1% L-glutamine without bFGF and placed into 96 well plates (200 μl/well and 5000 cells/well of cell suspension for each plate used). Three wells were kept empty for No Cell Controls. Cells were incubated overnight (24 h) at 37° C. in 5% CO₂. Next day, the media was removed by aspiration from each well, and 100 μl of sample was added to the wells in triplicate. Each sample was prepared at 2× the final concentration. Plates were incubated for 30 minutes at 37° C. in 5% CO₂. Each plate contained the following controls: For Standard Curve: Media alone with bFGF+Cells (3 wells) and Media with bFGF+Cells (9 wells). For Background: Media plus bFGF+cells and no label with BrdU (3 wells), media alone without bFGF and no label with BrdU (3 wells), and No Cells (3 wells). After 30 minutes of incubation, 100 μl of culture medium was added (M-200 supplemented with 2% heat inactivated FBS and 1% L-glutamine containing 4 ng/ml of bFGF). Final concentration of bFGF 2 was 2 ng/ml in all wells except for No bFGF controls. Cells were incubated for 72 h at 37° C. in 5% CO₂. Cells were then examined microscopically and assessed for viability. Cell proliferation was measured by the amount of BrdU incorporated during DNA synthesis.

### Example 16

Pre-Clinical Studies: In vitro, Proliferation, BrdU ELISA, HUVEC

Cell proliferation was measured by the amount of BrdU incorporation during DNA syntheses. The BrdU calorimetric ELISA kit was supplied by Roche Molecular Biochemicals Cat.# 1627 229.1. For labeling cells with BrdU, BrdU was diluted with labeling solution 1:100 in M-200 growth media (Cascade Cat. #M-200-500), supplemented with 2% heat inactivated FBS (Hyclone Cat. #SH30070) and 1% L-glutamine (BioWhittaker Cat. # 17-605E). To each well, 20 μl of BrdU was added, except for the wells containing the control, and incubated 2.5 h at 37° C. in 5% CO₂.

Cell fixation was carried out by first removing the label and growth medium from cells, drying the cells by blotting on paper towels, and adding 200 μl of FixDenat to each well. Cells were then incubated for 30 minutes at room temperature (RT), FixDenat was then removed and 100 ml of diluted anti-BrdU was added to each well and incubated for 2 h at room temperature. Anti-BrdU dilution was carried out in 1:100 in antibody dilution solution 5 minutes before addition to wells, and allow to reach the room temperature (RT). Anti-BrdU stock is lyophilized and were reconstituted before use in 1.1 ml of sterile tilled water. This stock was stored at 4° C.

After two hours incubation at room temperature, antibody conjugate was removed, cells were dried and washed. To rinse plates, 200 μl of washing solution was added to each well and wash was repeated 3×. The washing solution was prepared by diluting washing buffer 1:10 into sterile distilled water and adding 55 ml into 495 ml sterile water. The washing solution was stored at 4° C. After final wash, plates were dried and 100 ml of substrate solution was added to each well, incubated at room temperature until color development was sufficient (5-10 minutes). Light absorbance was measured at 370 nm-reference wavelength 492 nm. ELISA Reader was set at: L1(370 nm), L2 (492 nm); L1-L2, and plates were read at various time points: 5 min, 10 min and 20 min.

### Example 17

Pre-Clinical Studies: In vitro, Migration, Micro-Chemotaxis, HUVEC

Neuro Probe Standard 48 Well Chemotaxis Chamber Cat # AP48 (301-229-8598) was used in this experiment. Filter Membranes (Poretics Membrane Polycarbonate PVP Free 8 micron 25×80 mm (Osmonics Inc.800-444-8212Cat #10474) were coated with Rat tail Collagen Type 1 (BD Collaborative Res. Cat # 40236) at 100 μg/ml in 29 ml of 0.2N Acetic Acid. Collagen was diluted (100 μg/ml) in 0.2N Acetic Acid in 50 ml conical tube and mixed well. Collagen solution was placed in either a petridish or a small plastic staining box and membranes were submerged individually (10 membranes). The solution was agitated slowly on rocker at room temperature for 48 hours. Filters were air dried in laminar flow hood by laying out filters on open petri dishes. Store dry filters in covered container at room temperature (2 weeks).

### Example 18

Pre-Clinical Studies: Micro Chemotaxis Assay

HUVEC p2-p7 was harvested from flasks by trypsinization, and neutralized trypsin with growth medium. Trypsin/versene/media was removed by centrifugation at 1000 rpm for 5 minutes and resuspended in 10 ml of assay media (Medium 200 supplemented with 1% L-glutamine (BioWhittaker and 0.1% BSA) (Sigma Cat# A8412). Cells were counted by dilution with Trypan Blue solution, viability was determined and cells were then resuspended at 2×10⁵/ml assay media. Before adding cells to chambers, cells were preincubated with test proteins in 17×100 mm (14 ml) polypropylene round bottom tube (Falcon 35-2059) for 30 minutes at 37° C. with 5% CO₂. Controls were incubated with assay media. Chamber was prepared approximately 5 minutes before addition of cells. To the first three columns of the bottom chamber approximately 28 μl of assay media alone was added. This volume varies with individual chambers from 25-30 μl. Volumes were adjusted so that a slight positive menicus is formed over wells. Rest wells were filled with chemo-attractant (VEGF₁₆₅ R&D Cat #293-VE) 2-10 ng/ml. VEGF was reconstituted in assay media.

All manipulations with membranes were performed with forceps under sterile conditions. Gently placed silicone gasket on top of membrane, then, the top portion of the chamber, oriented with the trademark, was placed and firm even pressure was applied to screw on the thumb nuts tightly. Fifty μls of either control cells or treated cells were added to upper chambers. Chambers were placed in 150 mm petri dish with moist paper towel and incubated for 6 h at 37° C. with 5% CO₂. After about 6 hours, thumbs were gently removed. Filter membrane was fixed and stained with Diff-Quik (Dade Int. Cat# 84132-10), fixed for approximately 1-3 minutes (Solution I for 2 minutes; Solution II for 3 minutes). Membrane was rinsed 2× in distilled H₂O and placed on top of 3×2 glass microscope slide (VWR Cat# 28351-100). With a wet Kim-Wipe, non-migrating cells were removed while holding onto membrane to prevent movement. After removal of cells, the membrane was dried and 4 small drops of super-glue pipette tip was placed on each corner of the slide and viewed under microscope to determine total number of migrated cells. Total number of cells is assessed using computer imaging program.

### Example 19

Pre-Clinical Studies: In vitro, Capillary Morphogenesis, Endothelial Cell Cord Formation on Matrigel

Matrigel assay was performed according to the following procedure. Matrigel assay Media used was Medium 200 supplemented with LSGS (without hydrocortisone) and 5% heat inactivated FBS (Hyclone). Matrigel-coated plates were prepared by adding in each well 65 μl of thawed matrigel (thaw matrigel, phenol red free, Collaborative Biomedical Products: Cat # 356237) was added at 4° C. Cells were incubated for approximately 4 hours. Plates were incubated at 37° C. in 5% CO₂ for 30 minutes. HUVEC was harvested during incubation period. Human Umbilical Vein Endothelial Cells (HUVEC) were received frozen at passage 1 from Cascade Biologics, Inc., Portland, Oreg. (800-778-477) (Cat.# C-003-5C single donor). HUVEC p2-p7 was harvested from flasks by trypsinization. Trypsin was neutralized with growth medium (Medium-200: Endothelial cell Growth Medium (Cascade Cat. M-200-500) supplemented with LSGS (Cascade Cat. # S-003-10) and 0.1% L-Glutamine (BioWhittaker Cat.# 17605-E). Trypsin/versene/media was removed by centrifugation at 1000 rpm for 5 minutes and resuspended in 10 ml of assay media. Waning: do not over trypsinize cells or over centrifuge cells. Cells were counted by dilution with Trypan Blue solution and their viability was determined. Cells were then resuspended at 1×10⁵/ml in assay media. The assay was performed under sterile conditions, and 300 μl of each protein dilution was added to 300 μl of HUVEC suspension at 1×10⁵/ml and mixed gently. Protein/cell mixture (200 μl) was added into each matrigel-coated well (matrigel should be in gel form). Plates were incubated for 16 hours at 37° C. in 5% CO₂. After incubation, plates were examined microscopically and the number of junctions formed by the endothelial cells were counted. These wells were then counted via computerized imaging system.

### Example 20

Pre-Clinical Studies: In vitro, Apoptosis, Fluorescein-Based Tunel

Materials:

Apoptosis Kit: Promega Corporation: Apoptosis Detection System, Fluorescein, Cat # G3250. Becton Dickinson Labware: 2 Chamber Polystyrene Vessel, Tissue Culture Treated Glass Slide, Cat# 35-4102, Paraformaldehyde Methanol-free; RNAase-free (16% solution). DNase 1 Stock Solutions: 1M Tris-HCl (pH 7.9) 6.055 g of Tris-base (molecular weight 121.1 g/mol), 1 M NaCl, 2.922 g of NaCl, 1M MgCl₂ 1M CaCl₂ To make the DNase 1 buffer (10 ml) add the following amounts from the stocks: 0.4 ml of the 1M Tris-HCl (pH 7.9) 0.1 ml of the IM NaCl, 0.06 ml of the 1M MgCl₂, 0.1 ml of the IM CaCl₂. Preparation of Chamber Slides. Assay Media: Medium 200 supplemented with LSGS and 1% L-glutamine (BioWhittaker).

Methods:

HUVEC p2-p7 were harvested from flasks by trypsinization. Trypsin was neutralized with growth medium. Trypsin/versene/media was then removed by centrifugation at 1000 rpm for 5 minutes. Pellet was resuspended in 10 ml of assay media. Cells were counted by dilution with Trypan Blue solution and their viability was determined. Cells were then resuspended at 2.5×10⁴/ml for 50,000 cells/chamber and were plated (2 mls of cell suspension in each chamber). The chamber used was either pre-coated chamber BE cat# 35-4102 or 2 chamber polystyrene vessels that have been coated with 1-1.5% gelatin for 30 minutes at 37° C. Gelatin was removed and washed with PBS before the addition of cells. Cells were incubate overnight at 37° C. in 5% CO₂ in moist air.

The next day, cells were treated with test proteins as follows: Media was removed and replaced with 2 ml of fresh media. The chamber was incubated overnight (18 h) at 37° C. in 5% CO₂ in moist air. The next day, apoptosis assay was performed using Promega Fluorescein Apoptosis Kit # G3250. Media was removed from each chamber, cells were fixed (dilute stock of methanol-free formaldehyde 1:4 in PBS, add 0.5 ml 4% methanol-free formaldehyde 25 minutes at 4° C.). Cells were washed with PBS (0.5 ml) for 5 minutes twice and were permeabilized by immersing the slides in 0.2% Triton X-100 (100 μl in 50 ml PBS). Added 500 μl to each chamber for 5 minutes on ice. Triton X was aspirated and cells were washed with 2× PBS (1-2 mls) for 5 minutes. Two slides were taken to treat with DNase 1 as positive and negative controls. All other slides were left in PBS. Prepared 2 slides for positive and negative controls, and sample was treated with DNase 1.

Chamber was removed from the slide, excess liquid was removed and 100 μl of DNase 1 buffer was added to the fixed cells for 5 minutes at room temperature. Slides were treated with DNase 1 by adding 1 μl of stock DNase 1 (1 unit/l) to 1 ml of DNase 1 buffer. Added 100 μl of DNase 1 (1 unit/ml) to slide. Incubated for 10 minutes at room temperature, excess liquid was removed and slide was washed 3-4 times in deionized H₂O in Coplin jar. (This jar should be used for positive control only.)

Apoptosis Detection was continued as follows. Chamber was removed from slide, excess liquid was removed and covered the cells with 100 μl of equilibration buffer for 5-10 minutes at room temperature. While the cells were equilibrating, nucleotides were thawed out and mixed on ice, while being protected from light at all times. The TdT incubation buffer was used for all experimental and control reactions. (Total volume of 100 μl is used for each reaction.) For the negative control 45 μl of equilibration buffer was combined with 5 μl of nucleotide mix and 1 μl of sterile deionized water.

The following steps were carried out in the darkroom with red light: Tin-foiled petridish was used as a humidified chamber. Blotted carefully around the equilibrated areas with a Kim wipe added 100 μl of TdT incubation buffer, and covered with either coverslips or strips of parafilm. Slides were placed on top of moist paper towels in foiled petridish. The slides were incubate at 37° C. for 1.5 h. After 1.5 h incubation, plastic cover-slips were removed and the reaction was terminated by immersing the slides in 2× SSC in a Coplin jar for 15 minutes at room temperature. Samples were washed 3× by immersing the slides in PBS for 5 minutes at room temperature. Excess liquid was removed and a drop of Vectashield (approximately 25 μl) was added as a mounting medium with PI. Carefully sealed edges with clear nail polish before covering with coverslip, placed flat, and stored slides at 4° C. protected from the light.

The slides were then analyzed by counting the number of cells with green fluorescence within the nucleus of apoptotic cells (520 nm). Viewed red fluorescence of propidium iodide at >620 nm. (P1 stains both apoptotic and nonapoptotic cells red).

### REFERENCES

The following references are hereby incorporated by reference herein in their entirety.

Altschul, et al., J. Mol. Biol. 215:403-410 (1990).

Altschul et al., Nucleic Acids Res. 25:3389-3402 (1997).

Angiolillo, A. L., Sgadari, C., Taub, D. D., Liao, F., Farber, J. M., Miaheshwari, S., Kleinman, H. K., Reaman, G. H., and Tosato, G., Human interferon-inducible protein 10 is a potent inhibitor of angiogenesis in vivo., J. Exp. Med. 182:155-162 (1995).

Anthony, The Biochemistry of Methylotrophs 269 (1982).

Atherton, E. and Sheppard, R. C., Solid Phase Peptide Synthesis: A Practical Approach, IRL Press, Oxford England.

Auerbach et al., J. Microvasc. Res. 29:401-411 (1985).

Brake et al., Proc. Natl. Acad. Sci. USA 81:4642 (1984).

Brem et al., J. Neurosurg. 74:441-446 (1991).

Brenner and Lemer, Proc. Natl. Acad. Sci. USA 89:5381-5383 (1992).

Bunin et al., Proc. Natl. Acad. Sci. USA 91:4708-4712 (1994).

Cao, Y., Chen, C., Weatherbee, J. A., Tsang, M., and Folkman, J., Gro-beta, a C-X-C chemokine, is an angiogenesis inhibitor that suppresses the growth of Lewis lung carcinoma in mice, J. Exp. Med. 182:2069-2077 (1995).

Chakrabarty et al., Mol. Cell Biol. 5:3403 (1985).

Chen, C., Parangi, S., Tolentino, M. J., and Folkman, J., A strategy to discover circulating angiogenesis inhibitors generated by human tumors, Cancer Res. 55:4230-4233 (1995).

Christian et al., J. Mol. Biol. 227:711-718 (1992).

Clapp, C., Martial, J. A., Guzman, R. C., Rentier-Delrue, F., and Weiner, R. I., The 16-kilodalton N-terminal fragment of human prolactin is a potent inhibitor of angiogenesis, Endocrinology 133:1292-1299 (1993).

Clegg et al., Bio/Technol. 11:905-910 (1993).

Colman et al., Hemostasis and Thrombosis, J. B. Lippincott Company, 2:20 (1987). Curr. Top. Microbiol Immunol. 231:125 (1998).

Dameron, K. M., Volpert, O. V., Tainsky, M. A., and Bouck, N. (1994). Control of angiogenesis in fibroblasts by p53 regulation of thrombospondin-1. Science 265, 1582.

Dayhoff, et. al., Atlas of Protein Sequence and Structure, Nat'l Biomed. Res. Found., Washington, D.C. (1978).

Devlin et al., Science 249:404-406 (1990).

Ecker and Crooke, Bio/Technology 13:351-360 (1995).

Ellis et al., Mol. Cell. Biol. 5:1111 (1985).

Fields and Song, Nature 340:245-246 (1989).

Fishman et al, Medicine, 2d Ed., J. B. Lippincott Co., Philadelphia (1985).

Fleig et al., Gene 46:237 (1986).

Fodor et al., Science 251:767-773 (1991).

Folkman et al., Science 221:719-725 (1983).

Folkman, Advances in Cancer Research, eds. Klein and Weinhouse, Academic Press, New York, pp. 175-203 (1985)

Folkman, J. (1985). Angiogenesis and its inhibitors. In Important Advances in Oncology 1985, V. T. DeVita, S. Hellman, and S. Rosenberg, eds. (Philadelphia: J. B. Lippincott Company), pp. 42-62.

Folkman and Klagsbrun, Science 235:442-447 (1987).

Folkman, J., What is the evidence that tumors are angiogenesis dependent?, J. Natl. Cancer Inst. 82:4-6 (1989).

Folkman et al., N. Engl. J. Med. 333:1757-1763 (1995)

Folkman, J. (1996). Tumor angiogenesis and tissue factor. Nature Med. 2, 167-168.

Folkman, J., Haundenschild, C. C., and Zetter, B. R. (1979). Long-term culture of capillary endothelial cells. Proc. Natl. Acad. Sci. USA 76, 5217-5221.

Forsgren et al., FEBS 213:2, 254 (1987).

Fowlkes et al., BioTechniques 13:422-427 (1992).

Gavrieli, Y., Sherman, Y., and Ben-Sasson, S. A. (1992). Identification of programmed cell death in situ via specific labeling of nuclear DNA fragmentation. J. Cell Biol. 119, 493-501.

Genetic Engineering News, Apr. 15, 1994.

Gentz et al., Proc. Natl. Acad. Sci. 86:821-824 (1989).

Good, D. J., Polyerini, P. J., Rastinejad, F., Le Beau, M. M., Lemons, R. S., Frazier, W. A., and Bouck, N. P., A tumor suppressor-dependent inhibitor of angiogenesis is immunologically and functionally indistinguishable from a fragment of thrombospondin, Proc. Nat. Acad. Sci. USA. 87:6624-6628 (1990).

Grant, D. S., Tashiro, K. -I., Sequi-Real, B., Yamada, Y., Martin, G. R., and Kleinman, H. K. (1989). Two different laminin domains mediate the differentiation of human endothelial cells into capillary-like structures in vitro. Cell 58, 933-943.

Gross, J. L., Moscatelli, D., and Rifkin, D. B. (1983). Increased capillary endothelial cell protease activity in response to angiogenic stimuli in vitro. Proc. Natl. Acad. Sci. USA 80, 2623-2627.

Gupta, S. K., Hassel, T., and Singh, J. P., A potent inhibitor of endothelial cell proliferation is generated by proteolytic cleavage of the chemokine platelet factor 4, Proc. Natl. Acad. Sci. USA 92:7799-7803 (1995).

Hanlon et al., Cancer 83:130 (1998)

Hansson, et al., J. Mol. Biol. 287:265-76 (1999).

Harayama, Trends Biotechnol. 16(2):76-82 (1998).

Haseloff and Gerlach, Nature 334:585-591 (1988).

Hocke, Nature 320:275 (1986).

Hodges Int. J. Cancer 63:231 (1995)

Holmgren, L., O'Reilly, M. S., and Folkman, J. (1995). Dormancy of micrometastases: balanced proliferation and apoptosis in the presence of angiogenesis suppression. Nature Med. 1, 149-153.

Homandberg, G. A., Williams, J. E., Grant, D., B., S., and Eisenstein, R. (1985). Heparin-binding fragments of fibronectin are potent inhibitors of endothelial cell growth. Am. J. Path. 120, 327-332.

Hori, et al., Cancer Res. 51:6180-6184 (1991).

Houghten et al., Nature 354:84-86 (1991).

Invitrogen, San Diego, Calif, USA.

Janknecht et al., Proc. Natl. Acad. Sci. USA 88:8972-897 (1991).

Jones & Prakash, Yeast 6:363 (1990).

Kandel, J., Bossy-Wetzel, E., Radvany, F., Klagsbum, M., Folkman, J., and Hanahan, D., Neovascularization is associated with a switch to the export of bFGF in the multistep development of fibrosarcoma, Cell 66:1095-1104 (1991).

Karlin and Altschul, Proc. Natl. Acad. Sci. USA 87:2264-2268 (1990) modified as in Karlin and Altschul, Proc. Natl. Acad. Sci. USA 90:5873-5877 (1993).

Kim, K. J., Li, B., Winer, J., Armanini, M., Gillett, N., Phillips, H. S., and Ferrara, N., Inhibition of vascular endothelial growth factor-induced angiogenesis suppresses tumor growth in vivo, Nature 362, 841-844 (1993).

Kim, oncogen 17:3125 (1998).

Kubota et al., J. Cell Biol. 107:1589 (1988).

Langer, Science 249:1527-1533 (1990).

Lapthom, A. J. Harris, D. C. Littlejohn, A., Lustbader, J. W., Canfield, R. E, Machin, K. J., Morgan F. J and Isaacs, N. W., Crystal structure of human chorionic gonadotropin, Nature 369:455-461 (1994).

Levy et al., Science 228:190 (1985).

Lilia et al. Clin. Chem. 37:9 (1991).

Lorenzo and Blasco, Biotechniques 24(2):308-13 (1998).

Maione, T. E., Gray, G. S., Petro, J., Hunt, A. J., Donner, A. L., Bauer, S. I., Carson, H. F., and Sharpe, R. J., Inhibition of angiogenesis by recombinant human platelet factor-4 and related peptides, Science 247:77-79 (1990).

Maniatis et al., Molecular Cloning. A Laboratory Manual, Cold Spring Harbor Laboratory Press, Cold Spring Harbor, N. Y, USA (1982).

Mattheakis et al., Proc. Natl. Acad. Sci. USA 91:9022-9026 (1994).

McAleese et al., Eur. J. Biochem. 178: 413-417 (1988)

Millauer, B., Shawver, L. K., Plate, K. H., Risau, W., and Ullrich, A. (1994), Glioblastoma growth inhibited in vivo by a dominant-negative Flk-1 mutant, Nature 367:576-579 (1994).

Moses et al., Biotech. 9:630-634 (1991).

Muragaki, Y., Timmons, S., Griffith, C. M., Oh, S. P., Fadel, B., Quertemmous, T., and Olsen, B. -R. (1995). Mouse coll8a1 is expressed in a tissue-specific manner as three alternative variants and is localized in basement membrane zones. Proc. Natl. Acad. Sci. USA 92, 8763-8767.

Myers and Miller, CABIOS 4:11-17 (1988).

Nabel, et. al., PNAS, 96:324-326 (1999).

Nelson, J., Allen, W. E., Scott, W. N., Bailie, J. R., Walker, B., and McFerran, N. V. (1995). Murine epidermal growth factor (EGF) fragment (33-42) inhibits both EGF- and laminin-dependent endothelial cell motility and angiogenesis. Cancer Res. 55, 3772-3776.

Nguyen, M., Shing, Y., and Folkman, J. (1994). Quantitation of angiogenesis and antiangiogenesis in the chick embryo chorioallantoic membrane. Microvascular Res. 47, 31-40.

Oldenburg et al., Proc. Natl. Acad. Sci. USA 89:5393-5397 (1992).

Oliva D., Cali, L., Feo, S. and A. Giallongo (1991) Genomics 10: 157

O'Reilly, M. S., Holmgren, L., Chen, C. C., and Folkman, J. (1996). ANGIOSTATIN® induces and sustains dormancy of human primary tumors in mice. Nature Med. 2, 689-692.

O'Reilly, M. S., Holmgren, L., Shing, Y., Chen, C., Rosenthal, R. A., Moses, M., Lane, W. S., Cao, Y., Sage, E. H., and Follman, J., ANGIOSTATIN®: A novel angiogenesis inhibitor that mediates the suppression of metastases by a Lewis lung carcinoma, Cell 79:315-328 (1994).

O'Reilly, et al., Angiogenic Regulation of Metastatic Growth, Cell 79(2):315 (1994)

O'Reilly et al., Endostatin: An Endogeneous Inhibitor of Angiogenesis and Tumor Growth, Cell 88:277-285 (1997).

Obeso, J., Weber, J., and Auerbach. R. (1990). A hemangioendothelioma-derived cell line: its use as a model for the study of endothelial cell biology. Lab. Invest. 63, 259-269.

Oh, S. K., Kamagata, Y., Muragaki, Y., Timmons, S., Ooshima, A., and Olsen, B. R. (1994). Isolation and sequencing of cDNAs for proteins with multiple domains of GlyXaa-Yaa repeats identify a distinct family of collagenous proteins. Proc. Natl. Acad. Sci. USA 91, 4229-4233.

Ostresh et al., Proc. Natl. Acad. Sci. USA 91:11138-11142 (1994). Parangi, S., O'Reilly, M., Christofori, G., Holmgren, L., Grosfeld, J., Folkman, J., and Hanahan, D., Antiangiogenic therapy of transgenic mice impairs de novo tumor growth, Proc. Natl. Acad. Sci. USA 93:2002-2007 (1996).

Parmley and Smith, Adv. Exp. Med. Biol. 251:215-218 (1989).

Patten et al., Curr. Opinion Biotechnol. 8:724-33 (1997).

Patz, Am. J. Opthalmol. 94:715-743 (1982).

Ranger and Peppas, J., Macromol. Sci. Rev. Macromol. Chem. 23:61 (1983).

Rastinejad, F., Polyerini, P. J., and Bouck, N. P., Regulation of the activity of a new inhibitor of angiogenesis by a cancer suppressor gene, Cell 56:345-355 (1989).

Rehn, M., and Pihlajaniemi, T. (1994). al (XVIII), a collagen chain with frequent interruptions in the collagenous sequence, a distinct tissue distribution, and homology with type XV collagen. Proc. Natl. Acad. Sci. USA 91, 4234-4238.

Rehn, M., and Pihlajaniemi, T. (1995). Identification of three N-terminal ends of type XVIII collagen chains and tissue-specific differences in the expression of the corresponding transcripts. J. Biol. Chem. 270, 4705-4711.

Riegman, P H J, Vlietstra, R J, Suuirmeijer, L., Characterization of the human Kallikrein Locus, Genomics 14:6 (1992).

Rider and Taylor, Biochim, Biophys. Acta 365: 285-300 (1974)

Rogers, G. T., Biochim, Biophys. Acta, 695:227-249 (1981)

Ryan et al., Rec. Prog. Horm. Res. 43:383 (1987).

Sage, E. H., Bassuk, J. A., Vost, J. C., Folkman. M. J., and Lane, T. F. (1995). Inhibition of endothelial cell proliferation by SPARC is mediated through a Ca (2+)-binding EF-hand sequence. J. Cell Biochem. 57, 127-140.

Sakamato, N., Iwahana, M., Tanaka, N. G., and Osaka, 8. (1991). Inhibition of angiogenesis and tumor growth by a synthetic laminin peptide, CDPGYIGSR-NH₂. Cancer Res. 51, 903-906.

Sambrook et al., Molecular Cloning, A Laboratory Manual, Cold Spring Harbor Laboratory Press 1.101-1.104(1989).

Sarver et al, Science 247:1222-1225 (1990).

Schulze-Osthoff K, et al., Eur J Biochem 254:439 (1998)

Scopes, R., Protein Purification, Springer-Verlag, NY (1982).

Scott and Smith, Science 249:386-390 (1990).

Sefton, Biomed. Eng. 14:201 (1987).

Shackleford, K. A., MacKellar, W. C., Habeck, L. L, Zimmerman, R. E., Richardson, J. M., Gygi, C. M. Sutkowski, D. M., Becker, G. W., Neubsuer, B. L., and Mendelsohn, L. G. (1997). Biochemical properties and cellular effects of prostate-specific antigen (PSA). Proceedings of the American Association for Cancer Research. 38 428.

Shinzo Oikawa, Hiroshi Nakazato, and Goro Kosaki, Biochemical and Biophysical Res. Commu. 142: 511-518 (1987).

Sim et al., Angiogenesis 3:41(1999).

Simon et al., Proc. Natl. Acad. Sci. USA 89:9367-9371 (1992).

Sokoll, L. J. and Chan D. W. et al., Prostate-Specific Antigen Its Discovery and Biochemical Characteristics, Urologic Clinics of North America 24:253-259 (1997)

Span P N, Thomas C M, Thomas C M, Heuvel J J, Bosch R R, Bosch R R, Schalken J A, vd Locht L, Mensink E J, Sweep C G, J Endocrinol 172(3):489-95 (2002)

Strieter, R. M., Kunkel, S. L., Arenberg, D. A., Burdick, M. D., and Polyerini, P. J., Human interferon-inducible protein 10 (IP-10), a member of the C-X-C chemokine family, is an inhibitor of angiogenesis, Biochem. Biophys. Res. Comm. 210:51-57 (1995).

Stockell et al., Biochem. J. 287:665 (1992)

Studier, W. F., Rosenberg, A. H., Dunn, J. J., and Dudendorf, J. W. (1990). Use of T7 RNA polymerase to direct expression of cloned genes. Methods Enzymol. 85, 60-89.

Teicher, B. A., Holden, S. A., Ara, G., Sotomayor, E. A., and Dong, H. Z. (1994). Potentiation of cytotoxic cancer therapies by TNP-470 alone and with other antiangiogenic agents. Int. J. Cancer 57, 1-6.

Thomer et al., The Molecular Biology the Yeast Saccharomyces, Cold Spring Harbor Laboratory, Cold Spring Harbor, N.Y. 143 (1981).

Tolsma, S. S., Volpert, O. V., Good, D. J., Frazier, W. A., Polyerini, P. J., and Bouck, N. (1993). Peptides derived from two separate domains of the matrix protein thrombospondin-1 have antiangiogenic activity. J. Cell Biol.122, 497-511.

Vedvick et al., J. Ind. Microbiol. 7:197-201 (1991).

Voest, E. E., Kenyon, B. M., O'Reilly, M. S., Truitt, G., D'Amato, R. J., and Folkman, J., Inhibition of angiogenesis in vivo by interleukin 12, J. Natl. Cancer Inst. 87:581-586 (1995).

Waltman et al, Am. J. Ophthal. 85:704 (1978).

Wilson et al., Cell 37:767 (1984)

Wu and Wu, J. Biol. Chem. 262:4429-4432 (1987).

Yang N., Gene Transfer into Mammalian Somatic Cells in vivo, Crit. Rev. Biotechn. 12(4): 335-356 (1992).

Yates et al., Nature 313:812 (1985).

Yu et al., Cell 76:933-945 (1994).

Zomzely-Neurath (1983). Enolase. In ‘Handbook of Neurochemistry” (A. Lajtha, Ed.), Vol. 4, 2nd ed., pp. 403-433, Plenum Press, New York.)).

We claim:

1. A method of treating, ameliorating, or preventing a cell proliferative disease or disorder in a mammal in need thereof comprising administering to the mammal an effective amount of a composition comprising a cancer marker and a pharmaceutically acceptable carrier or diluent.

2. The method of claim 1, wherein the cancer marker comprises prostate specific antigen (PSA), human chorionic gonadotropin (HCG-α and HCG-β, alpha-fetoprotein, carcinoembryonic antigen (CEA), neuron specific enolase (NSE), squamous cell carcinoma-associated antigen (SCC), cancer antigen (CA)125, CA15-3, CA19-9, CD20, CDH13, CD31, CD34, CD105, CD146, D16S422HER-2, phospatidylinositol 3-kinase (PI 3-kinase), trypsin, trypsin-1 complexed with alpha(1)-antitrypsin, estrogen receptor, progesterone receptor, c-erbB-2, bcl-2, S-phase fraction (SPF), p185erbB-2, low-affmity insulin like growth factor-binding protein, urinary tissue factor, vascular endothelial growth factor, epidermal growth factor, epidermal growth factor receptor, apoptosis proteins (Ki67), factor VIII, adhesion proteins (CD-44, sialyl-TN, blood group A), human placental alkaline phosphatase (ALP), thymidine phosphorylase (dTHdPase), thrombomodulin, laminin receptor, fibronectin, anticyclins, anticyclin A, B, or E, proliferation associated nuclear antigen, lectin UEA-1, von Willebrand's factor, or a combination thereof.

3. The method of claim 1, wherein said cancer marker comprises PSA, CEA, HCG-α, HCG-β, NSE, CA 19-9, or a combination thereof.

4. The method of claim 1, wherein the composition further comprises an angiogenesis inhibitory polypeptide, a cytotoxic agent, or both.

5. The method of claim 4, wherein the angiogenesis inhibiting polypeptide comprises angiostatin, endostatin, or both.

6. The method of claim 1, wherein said cell proliferative disease or disorder is an angiogenesis-related disease or disorder.

7. The method of claim 6, wherein said angiogenesis-related disease or disorder is cancer and said pharmaceutical composition inhibits growth, progression, and/or metastasis of cancer.

8. The method of claim 7, wherein said cancer comprises solid tumors.

9. The method of claim 6, wherein said angiogenesis-related disease or disorder comprises benign tumors.

10. The method of claim 7, wherein said angiogenesis-related disease or disorder comprises neovascular diseases of the eye.

11. The method of claim 6, wherein said cancer marker increases apoptosis.

12. A method of screening a compound for its ability to regulate angiogenesis comprising:
(a) identifying a candidate cancer marker;
(b) preparing said cancer marker for testing; and
(c) testing said cancer marker in at least one bioassay to determine an inhibitory affect of said cancer marker on endothelial cell formation and/or proliferation, wherein an inhibitory effect of said cancer marker in said at least one bioassay correlates with angiogenesis inhibitory activity of said cancer marker.

13. The method of claim 12, wherein said at least one bioassay comprises a proliferation assay, migration assay, invasion assay, cord formation assay, apoptosis assay, or a combination thereof.

14. The method of claim 12, wherein said at least one bioassay comprises a human umbilical vein endothelial cell proliferation assay (HUVEC), bovine capillary endothelial cell proliferation assay (BCE), chick CAM assay, mouse corneal assay, matrigel assay, implanted tumor assay, or a combination thereof.

15. The method of claim 12, wherein said preparing cancer marker of step (b) comprises isolating from a patient's sample, chemical synthesis, or recombinant production of the cancer marker.

16. The method of claim 12, further comprising the steps of:
(d) contacting said cancer marker with a plurality of molecules; and
(e) identifying a molecule that binds said cancer marker in order to obtain a binding molecule,
wherein said binding molecule has an increased angiogenesis regulatory activity, as compared to said cancer marker.

17. A pharmaceutical composition for protection, amelioration, or inhibition of a cell proliferative disease or disorder, comprising a cancer marker and a pharmaceutically acceptable carrier or diluent, wherein said cancer marker inhibits endothelial cell formation and/or proliferation in at least one bioassay in vitro.

18. The pharmaceutical composition of claim 18, wherein said cancer marker comprises PSA, CEA, HCG-α, HCG-β, NSE, CA 19-9, or a combination thereof.

19. The pharmaceutical composition of claim 18, additionally containing an angiogenesis inhibitory polypeptide, a cytotoxic agent, or both.

20. The pharmaceutical composition of claim 20, wherein said angiogenesis inhibitory polypeptide comprises angiostatin, endostatin, or both.
